(19) United States
(12) Patent Application Publication
(10) Pub. No.: US 2011/0190285 A1
(43) Pub. Date: Aug. 4, 2011

(54) METHODS OF TREATING A BOTULINUM TOXIN RELATED CONDITION IN A SUBJECT

(75) Inventors: Alan Jacobson, Denver, CO (US); Scott Moe, Sudbury, MA (US)

(73) Assignee: ABSOLUTE SCIENCE, INC., Denver, CO (US)

(21) Appl. No.: 13/063,078

(22) PCT Filed: Aug. 26, 2009
(86) PCT No.: PCT/US09/55061

§ 371 Date: Apr. 20, 2011

Related U.S. Application Data
(60) Provisional application No. 61/103,394, filed on Oct. 7, 2008.

(30) Foreign Application Priority Data
Sep. 19, 2008 (US) PCT/US2008/077073

(51) Int. Cl. A61K 31/167; A61K 31/277; A61K 31/44; A61K 31/428; A61K 31/415; A61K 31/505; A61K 31/473; A61K 31/502; A61K 31/5375; A61P 43/00

(52) U.S. Cl. 514/231.2; 514/522; 514/625; 514/349; 514/367; 514/407; 514/256; 514/297; 514/248

(57) ABSTRACT

The present invention provides methods of treating a botulinum toxin related condition in a subject. In certain embodiments, the methods involve administering a compound of the following formulas: (I), (II), (III), (IV), (V).

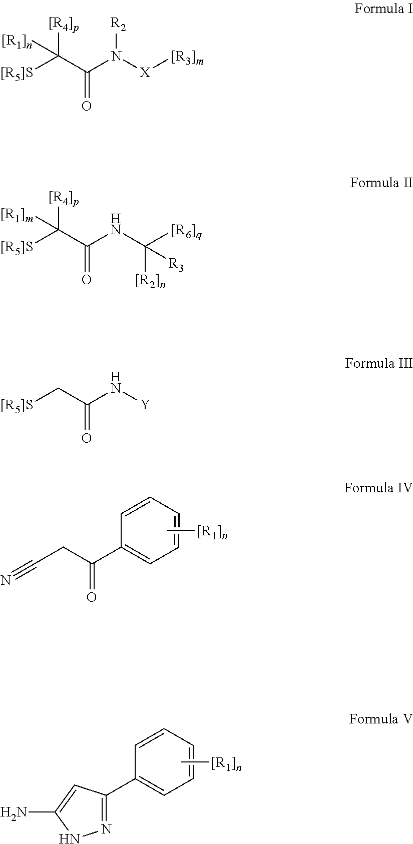

### RELATED APPLICATION

This application claims the benefit of and is related to U.S. provisional patent application Ser. No. 61/103,394, which was filed in the U.S. Patent and Trademark Office Oct. 7, 2008. This application also claims the benefit of and priority to PCT international patent application number PCT/US08/77073, which was filed in the Receiving Office of the U.S. Patent and Trademark Office Sep. 19, 2008, which claims the benefit of and priority to U.S. provisional patent application Ser. No. 60/995,769 which was filed in the U.S. Patent and Trademark Office Sep. 28, 2007. The contents of each of which is incorporated by reference herein in its entirety.

### GOVERNMENT SUPPORT

The U.S. Government has a paid-up license in this invention and the right in limited circumstances to require the patent owner to license others on reasonable terms as provided for by the terms of Contract Number N01-AI-30050 awarded by NIAID, NIH and DHHS.

### TECHNICAL FIELD

The present invention provides methods of treating botulinum toxin related condition in a subject, and compounds that are useful as inhibitors of botulinum intoxication.

### BACKGROUND

Botulinum toxin is a neurotoxin protein produced by the bacterium Clostridium botulinum. This protein is considered extremely toxic and is one of the most poisonous naturally occurring substances. The basis of potency of botulinum toxin is enzymatic; the toxin is a zinc protease that cleaves one or more of the fusion proteins by which neuronal vesicles release acetylcholine into the neuromuscular junction (Arnon et al., JAMA. 2001; 285:1059-1070).

There are three main kinds of botulism. Foodborne botulism is caused by eating foods that contain the botulism toxin. Wound botulism is caused by toxin produced from a wound infected with Clostridium botulinum. Infant botulism is caused by consuming the spores of the botulinum bacteria, which then grow in the intestines and release toxin. Symptoms of botulism include paralysis, double vision, blurred vision, drooping eyelids, slurred speech, difficulty swallowing, dry mouth, and muscle weakness.

Botulinum toxin poses a concern because of its potency; its ease of production, transport, and misuse; and the need for prolonged intensive care among affected persons (Franz et al. JAMA. 1997; 278:399-411). Botulism infection can constitute a medical situation that requires prompt provision of botulinum antitoxin and, often, mechanical ventilation, and requires quick intervention to prevent additional cases (Arnon et al., JAMA. 2001; 285:1059-1070).

Therapy for botulism consists of supportive care and passive immunization with equine antitoxin, i.e., antibody treatment. Optimal use of botulinum antitoxin requires early suspicion of botulism, and timely administration of the antitoxin to minimize subsequent nerve damage and severity of disease (Tacket et al. Am J. Med. 1984; 794-798). Current therapies are unable to reverse existent paralysis.

There is a need for methods of treating botulinum toxin and active inhibitory synthetic compounds that are suitable for treating a botulinum toxin related condition, such as but not limited to botulism, and that are stable, efficacious, and specific with minimal side effects.

### SUMMARY

The present invention provides compositions that are useful for treating a subject afflicted with a botulinum toxin related condition, e.g., botulism, and methods of treating a botulinum related condition. An aspect of the invention provides a method for treating a botulinum toxin related condition comprising administering to a subject, for example a human or a non-human subject, in need thereof a therapeutically effective amount of a pharmaceutical composition comprising a mercaptoacetamide. Exemplary non-human subjects include fish or fowl. In certain embodiments of the method, the botulinum toxin is a serotype selected from the group consisting of: serotype A, serotype B, serotype C, serotype D, serotype E, serotype F and serotype G.

In certain embodiments of the method, the mercaptoacetamide is a compound of Formula I:

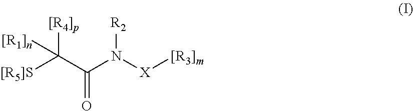

in which: X is a C₃-C₆ heterocycloalkenyl, wherein the atoms of the ring are optionally substituted by R₆, and wherein when one or more heteroatoms are nitrogen, the nitrogens are each independently unsubstituted or substituted by R₇; R₁ is present at n occurrences, n is an integer from 0 to 1, and R₁ is selected from H, C₂-C₆ alkenyl, C₂-C₆ alkynyl, C(═O)OR₉, and C₁-C₆ alkyl optionally substituted by R₈; R₂ is selected from H, C₂-C₆ alkenyl, C₂-C₆ alkynyl, C(═O)OR₉, and C₁-C₆ alkyl optionally substituted by R₈; R₃ is present at m occurrences, m is an integer from 0 to 1, and R₃ is selected from a proton, C(═O)OR₁₀, C(═O)OR₇, C(═O)NR₇, C₃-C₆ heterocycloalkylaryl, C₃-C₆ cycloalkenylaryl, C—R₈, heteroaryl, and aryl optionally substituted at each carbon atom by halo, OH, OCH₃, O-alkyl, amino, substituted amino, —SO₂NH₂, substituted sulfonamide, —SO₂CH₃, substitutied sulfoxies, CONH₂, substituted amido, COCH₃, substituted ketones, CHO, cyano, NO₂, C(═O)OR₁₀, and C₁-C₆ alkyl which is further optionally substituted by halo, amino, or hydroxyl; R₄ is present at p occurrences, p is an integer from 0 to 1, and R₄ is selected from H, C₂-C₆ alkenyl, C₂-C₆ alkynyl, C(═O)OR₉, and C₁-C₆ alkyl optionally substituted by R₈; R₅ is a hydrogen atom, or a bond such that the molecule formed a symmetrical dimer at the disulfide bond, a mixed disulfide with other monosulfide compounds such as ethanethiol, or functional groups such as acetyl to form esters which can be used as prodrugs, H, —C(═O)R₉, and —C(═O)OR₉; R₆ is selected from H, C₂-C₆ alkenyl, C₂-C₆ alkynyl, C(═O)OR₉, and C₁-C₆ alkyl optionally substituted by R₈; R₇ is selected from H, C₁-C₆ alkyl optionally substituted by R₈; C₂-C₆ alkenyl, C₂-C₆ alkynyl, C(═O)OR₉, and aryl optionally substituted by halo or C₁-C₆ alkyl; R₈ is selected from C(═O)OR₉, OR₉, and halo; R₉ is a C₁-C₆ alkyl optionally substituted by aryl; R₁₀ is selected from H, halo, OR₉, OH, NO₂, NH₂, alkoxy, cyano, SO₂CH₃, SO₂NH₂, COCH₃, COCH₃, CONH₂, CHO and C₁-C₆ alkyl optionally substituted by halo; R₁₁ is an aryl optionally substituted by halo; or pharmaceutically acceptable salts and prodrugs thereof.

In related embodiments of the method, the compound of Formula I includes the following structure: X is a C₃-C₆ heterocycloalkenyl, wherein carbon atoms of the ring are optionally substituted by R₆, and wherein when one or more heteroatoms are nitrogen, the nitrogens are each independently unsubstituted or substituted by R₇; R₁ and R₂ are independently selected from H or methyl; is selected from a proton, C(═O)OR₁₀, C(═O)OR₇, C(═O)NR₇, C₃-C₆ heterocycloalkylaryl, C₃-C₆ cycloalkenylaryl, C—R₈, heteroaryl, and aryl optionally substituted at each carbon atom by halo, OH, OCH₃, O-alkyl, amino, substituted amino, —SO₂NH₂, substituted sulfonamide, —SO₂CH₃, substitutied sulfoxies, CONH2, substituted amido, COCH₃, substituted ketones, CHO, cyano, NO₂, C(═O)OR₁₀, and C₁-C₆ alkyl which is further optionally substituted by halo, amino, or hydroxyl; R₆ and R₇ are independently selected from H or methyl; or pharmaceutically acceptable salt and prodrugs thereof.

In certain embodiments, X is at least one compound selected from the group consisting of pyrazole, thiazole, and thiadiazole. In a related embodiment, the pyrazole is a 1,2 pyrazole. In another related embodiment, the thiazole is a 1,3 thiazole. In yet another related embodiment, the thiadiazole is a 4-thia-1,2 diazole.

In another embodiment of the method, the compound of Formula I includes the following structure: R₁ and R₂ are both H. In another embodiment of the method, the compound of Formula I includes the following structure: R₆ and R₇ are both H. In another embodiment of the method, the compound of Formula I includes the following structure: R₃ is at least one compound selected from the group consisting of phenyl, furyl, pyridyl, and thiophene. In a related embodiment, the thiophene is 2-thiophene.

In other embodiments of the method, the mercaptoacetamide is a compound of Formula II:

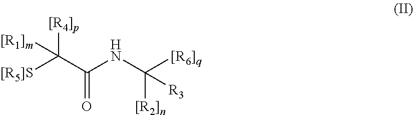

in which: R₁ is present at m occurrences, m is an integer from 0 to 1, and R₁ is C₁-C₆ alkyl or C—R₈, R₂ is present at n occurrences, n is an integer from 0 to 1, and R₂ is selected from a proton, C₁-C₆ alkyl, C(═O)OR₇, alkyl-OR₇, C—R₈, and alkyl-NR_{S}; is selected from a proton, C(═O)OR₁₀, C(═O)OR₇, C(═O)NR₇, C₃-C₆ heterocycloalkylaryl, C₃-C₆ cycloalkenylaryl, C—R₈, heteroaryl, and aryl optionally substituted at each carbon atom by halo, OH, OCH₃, O-alkyl, amino, substituted amino, —SO₂NH₂, substituted sulfonamide, —SO₂CH₃, substitutied sulfoxies, CONH₂, substituted amido, COCH₃, substituted ketones, CHO, cyano, NO₂, C(═O)OR₁₀, and C₁-C₆ alkyl which is further optionally substituted by halo, amino, or hydroxyl; R₄ is present at p occurrences, p is an integer from 0 to 1, and R₄ is C₁-C₆ alkyl or C—R₈, R₅ is a hydrogen atom, or a bond such that the molecule formed a symmetrical dimer at the disulfide bond, a mixed disulfide with other monosulfide compounds such as ethanethiol, or functional groups such as acetyl to form esters which can be used as prodrugs, H, and —C(═O)R₁₀; R₆ is present at q occurrences, q is an integer from 0 to 1, and R₆ is aryl; R₇ is selected from C—R₈; R₈ is selected from C₃-C₆ cycloalkenylaryl, C₃-C₆ heterocycloalkenylaryl, and aryl optionally substituted by OH, aryl, or OR₁₀; R₉ is C(═O)OR₁₀; R₁₀ is C₁-C₆ alkyl; or pharmaceutically acceptable salts and prodrugs thereof.

In other embodiments of the method, the mercaptoacetamide is a compound of Formula III:

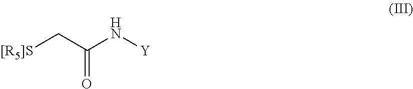

in which: Y is selected from C₁-C₆ alkyl, C₂-C₆ alkenyl, C₃-C₆ cycloalkenylaryl, C₃-C₆ heterocycloalkenylaryl, C₃-C₆ cycloalkylaryl, C₃-C₆ heterocycloalkylaryl, aryl, heteroaryl, C₃-C₆ heterocycloalkenyl, C₃-C₆ arylcycloalkylaryl, any of which is optionally substituted at each carbon atom by R₁, and wherein when one or more heteroatoms are nitrogen, the nitrogens are each independently unsubstituted or substituted by R₂; R₁ is selected from OH, cyano, SH, halo, alkyl-NR₂R₃, OR₂, aryl, oxo, C—R₃, OR₃, C₂-C₆ alkynyl, C₃-C₆ heterocycloalkenylaryl, C₁-C₆ alkyl optionally substituted by halo; and C₃-C₆ heterocycloalkenyl which is optionally substituted at each carbon atom by R₄; R₂ is selected from C₁-C₆ alkyl; is selected from a proton, C(═O)ORᵢₒ, C(═O)OR₇, C(═O)NR₇, C₃-C₆ heterocycloalkylaryl, C₃-C₆ cycloalkenylaryl, C—R₈, heteroaryl, and aryl optionally substituted at each carbon atom by halo, OH, OCH₃, O-alkyl, amino, substituted amino, —SO₂NH₂, substituted sulfonamide, —SO₂CH₃, substitutied sulfoxies, CONH2, substituted amido, COCH₃, substituted ketones, CHO, cyano, NO₂, C(═O)OR₁₀, and C₁-C₆ alkyl which is further optionally substituted by halo, amino, or hydroxyl; R₄ is C(═O)OR₂; R₅ is a hydrogen atom, or a bond such that the molecule formed a symmetrical dimer at the disulfide bond, a mixed disulfide with other monosulfide compounds such as ethanethiol, or functional groups such as acetyl to form esters which can be used as prodrugs; or pharmaceutically acceptable salts and prodrugs thereof.

In certain embodiments of the methods, the above compounds include a structure in which R₅ is a hydrogen atom, or a bond such that the molecule formed a symmetrical dimer at the disulfide bond, a mixed disulfide with other monosulfide compounds such as ethanethiol, or functional groups such as acetyl to form esters which can be used as prodrugs.

Another aspect of the invention provides a method for treating a botulinum toxin related condition comprising administering to a subject in need thereof a therapeutically effective amount of a pharmaceutical composition comprising a compound of Formula IV:

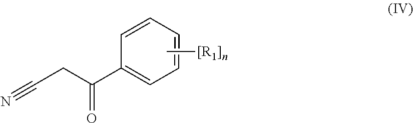

in which: R₁ is present at n occurrences, n is an integer from 0 to 5 and R₁ is selected from halo and C₁-C₆ alkyl optionally substituted by halo.

Another aspect of the invention provides a method for treating a botulinum toxin related condition comprising administering to a subject in need thereof a therapeutically effective amount of a pharmaceutical composition comprising a compound of Formula V:

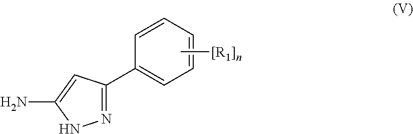

in which: R₁ is present at n occurrences, n is an integer from 0 to 5 and R₁ is selected from halo and C₁-C₆ alkyl optionally substituted by halo.

Another aspect of the invention provides a pharmaceutical composition including a mercaptoacetamide in a dosage effective to treat a botulinum toxin related condition, and a pharmaceutically acceptable carrier.

### DETAILED DESCRIPTION

The invention provides methods of treating a botulinum toxin related condition, e.g., botulism, in a subject by administering a compound comprising a mercaptoacetamide, methods for the manufacture of pharmaceutical compositions for use in the treatment of these diseases, and pharmaceutical preparations having compounds of the present invention for the treatment of these diseases are also provided.

In certain embodiments, the mercaptoacetamide is a compounds of Formula I,

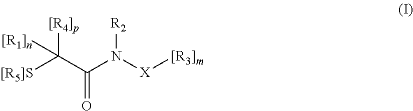

in which: X is a C₃-C₆ heterocycloalkenyl, wherein carbon atoms of the ring are optionally substituted by R₆, and wherein when one or more heteroatoms are nitrogen, the nitrogens are each independently unsubstituted or substituted by R₇; R₁ is present at n occurrences, n is an integer from 0 to 1, and R₁ is selected from H, C₂-C₆ alkenyl, C₂-C₆ alkynyl, C(═O)OR₉, and C₁-C₆ alkyl optionally substituted by R₈; R₂ is selected from H, C₂-C₆ alkenyl, C₂-C₆ alkynyl, C(═O)OR₉, and C₁-C₆ alkyl optionally substituted by R₈; R₃ is present at m occurrences, m is an integer from 0 to 1, and R₃ is selected from a proton, C(═O)OR₁₀, C(═O)OR₇, C(═O)NR₇, C₃-C₆ heterocycloalkylaryl, C₃-C₆ cycloalkenylaryl, C—R₈, heteroaryl, and aryl optionally substituted at each carbon atom by halo, OH, OCH₃, O-alkyl, amino, substituted amino, —SO₂NH₂, substituted sulfonamide, —SO₂CH₃, substitutied sulfoxies, CONH₂, substituted amido, COCH₃, substituted ketones, CHO, cyano, NO₂, C(═O)OR₁₀, and C₁-C₆ alkyl which is further optionally substituted by halo, amino, or hydroxyl; R₄ is present at p occurrences, p is an integer from 0 to 1, and R₄ is selected from H, C₂-C₆ alkenyl, C₂-C₆ alkynyl, C(═O)OR₉, and C₁-C₆ alkyl optionally substituted by R₈; R₅ is a hydrogen atom, or a bond such that the molecule formed a symmetrical dimer at the disulfide bond, a mixed disulfide with other monosulfide compounds such as ethanethiol, or functional groups such as acetyl to form esters which can be used as prodrugs, H, —C(═O)R₉, and —C(═O)OR₉; R₆ is selected from H, C₂-C₆ alkenyl, C₂-C₆ alkynyl, C(═O)OR₉, and C₁-C₆ alkyl optionally substituted by R₈; R₇ is selected from H, C₁-C₆ alkyl optionally substituted by R₈; C₂-C₆ alkenyl, C₂-C₆ alkynyl, C(═O)OR₉, and aryl optionally substituted by halo or C₁-C₆ alkyl; R₈ is selected from C(═O)OR₉, and halo; R₉ is a C₁-C₆ alkyl optionally substituted by aryl; R₁₀ is selected from H, halo, OR₉, NO₂, alkoxy, cyano, SO₂CH₃, SO₂NH₂, COCH₃, COCH₃, CONH₂, CHO and C₁-C₆ alkyl optionally substituted by halo; and R₁₁ is an aryl optionally substituted by halo.

In alternative embodiments, the mercaptoacetamide is a compounds having Formula II,

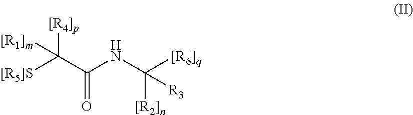

in which: R₁ is present at m occurrences, m is an integer from 0 to 1, and R₁ is C₁-C₆ alkyl or C—R₈, R₂ is present at n occurrences, n is an integer from 0 to 1, and R₂ is selected from a proton, C₁-C₆ alkyl, C(═O)OR₇, alkyl-OR_{S}, C—R₈, and alkyl-NR₉; R₃ is selected from a proton, C(═O)OR₁₀, C(═O)OR₇, C(═O)NR₇, C₃-C₆ heterocycloalkylaryl, C₃-C₆ cycloalkenylaryl, C—R₈, heteroaryl, and aryl optionally substituted at each carbon atom by halo, OH, OCH₃, O-alkyl, amino, substituted amino, —SO₂NH₂, substituted sulfonamide, —SO₂CH₃, substitutied sulfoxies, CONH₂, substituted amido, COCH₃, substituted ketones, CHO, cyano, NO₂, C(═O)OR₁₀, and C₁-C₆ alkyl which is further optionally substituted by halo, amino, or hydroxyl; R₄ is present at p occurrences, p is an integer from 0 to 1, and R₄ is C₁-C₆ alkyl or C—R₈; R₅ is a hydrogen atom, or a bond such that the molecule formed a symmetrical dimer at the disulfide bond, a mixed disulfide with other monosulfide compounds such as ethanethiol, or functional groups such as acetyl to form esters which can be used as prodrugs, H, and —C(═O)R₁₀; R₆ is present at q occurrences, q is an integer from 0 to 1, and R₆ is aryl; and R₇ is selected from C—R₈; R₈ is selected from C₃-C₆ cycloalkenylaryl, C₃-C₆ heterocycloalkenylaryl, and aryl optionally substituted by OH, aryl, or OR₁₀; R₉ is C(═O)OR₁₀; R₁₀ is C₁-C₆ alkyl.

In another embodiment, the mercaptoacetamide is a compound having Formula III,

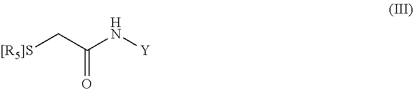

in which: Y is selected from C₁-C₆ alkyl, C₂-C₆ alkenyl, C₃-C₆ cycloalkenylaryl, C₃-C₆ heterocycloalkenylaryl, C₃-C₆ cycloalkylaryl, C₃-C₆ heterocycloalkylaryl, aryl, heteroaryl, C₃-C₆ heterocycloalkenyl, C₃-C₆ arylcycloalkylaryl, any of which is optionally substituted at each carbon atom by R₁, and wherein when one or more heteroatoms are nitrogen, the nitrogens are each independently unsubstituted or substituted by R₂; R₁ is selected from OH, cyano, SH, halo, alkyl-NR₂R₃, OR₂, aryl, oxo, C—R₃, OR₃, C₂-C₆ alkynyl, C₃-C₆ heterocycloalkenylaryl, C₁-C₆ alkyl optionally substituted by halo; and C₃-C₆ heterocycloalkenyl which is optionally substituted at each carbon atom by R₄; R₂ is selected from C₁-C₆ alkyl; R₃ is selected from a proton, C(═O)OR₁₀, C(═O)OR₇, C(═O)NR₇, C₃-C₆ heterocycloalkylaryl, C₃-C₆ cycloalkenylaryl, C—R₈, heteroaryl, and aryl optionally substituted at each carbon atom by halo, OH, OCH₃, O-alkyl, amino, substituted amino, —SO₂NH₂, substituted sulfonamide, —SO₂CH₃, substitutied sulfoxies, CONH₂, substituted amido, COCH₃, substituted ketones, CHO, cyano, NO₂, C(═O)OR₁₀, and C₁-C₆ alkyl which is further optionally substituted by halo, amino, or hydroxyl; R₄ is C(═O)OR₂; and R₅ is a hydrogen atom, or a bond such that the molecule formed a symmetrical dimer at the disulfide bond, a mixed disulfide with other monosulfide compounds such as ethanethiol, or functional groups such as acetyl to form esters which can be used as prodrugs.

In yet another embodiment, the invention provides treating a subject having a botulinum toxin related condition by administering compounds of Formula IV,

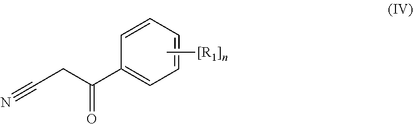

in which: R₁ is present at n occurrences, n is an integer from 0 to 5 and R₁ is selected from halo and C₁-C₆ alkyl optionally substituted by halo.

In still another embodiment, the invention provides treating a subject having a botulinum toxin related condition by administering compounds of Formula V,

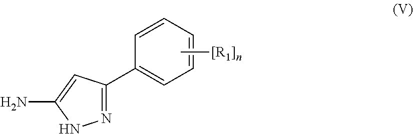

in which: R₁ is present at n occurrences, n is an integer from 0 to 5 and R₁ is selected from halo and C₁-C₆ alkyl optionally substituted by halo.

The following are exemplary compounds of Formulas I-V:

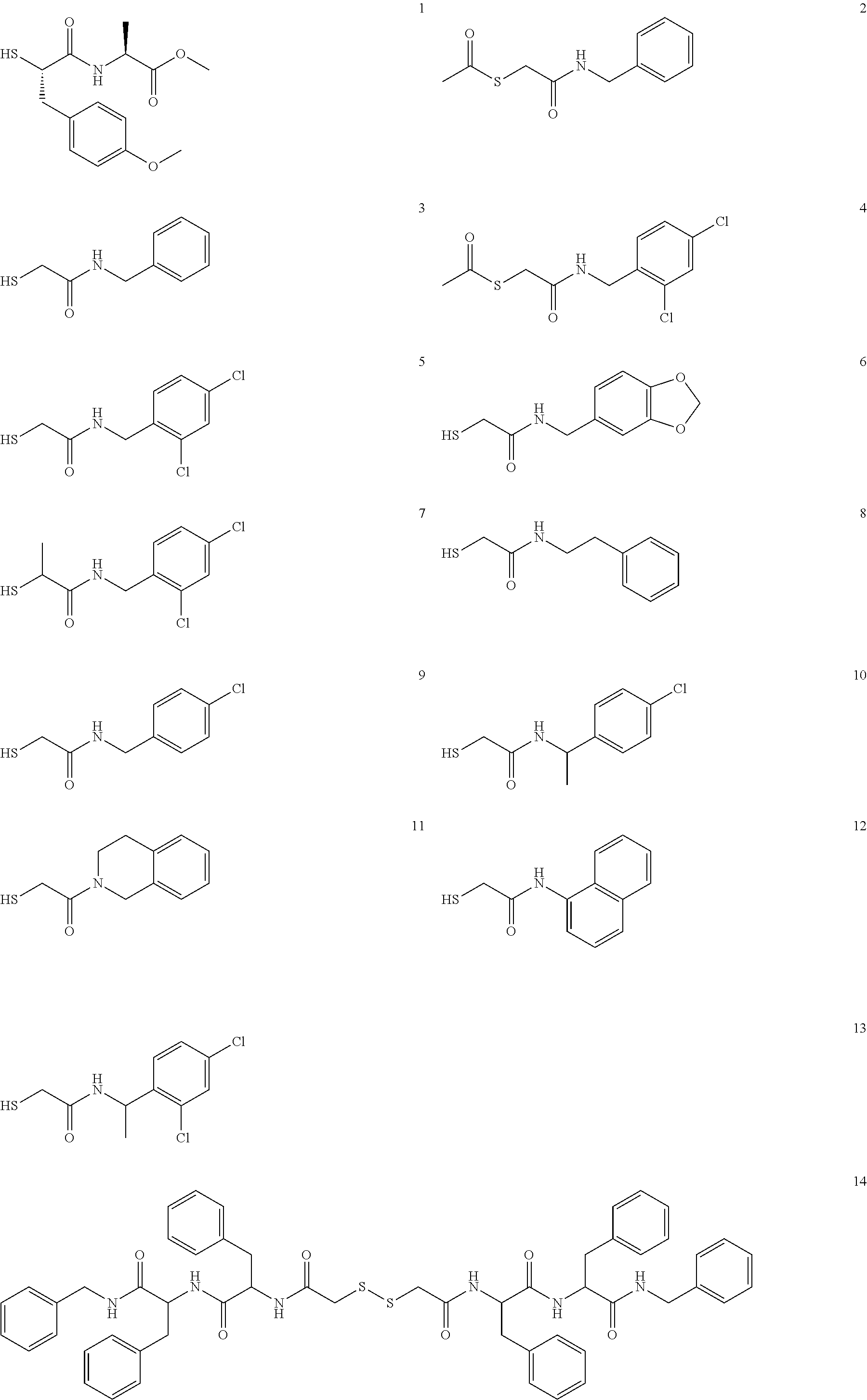

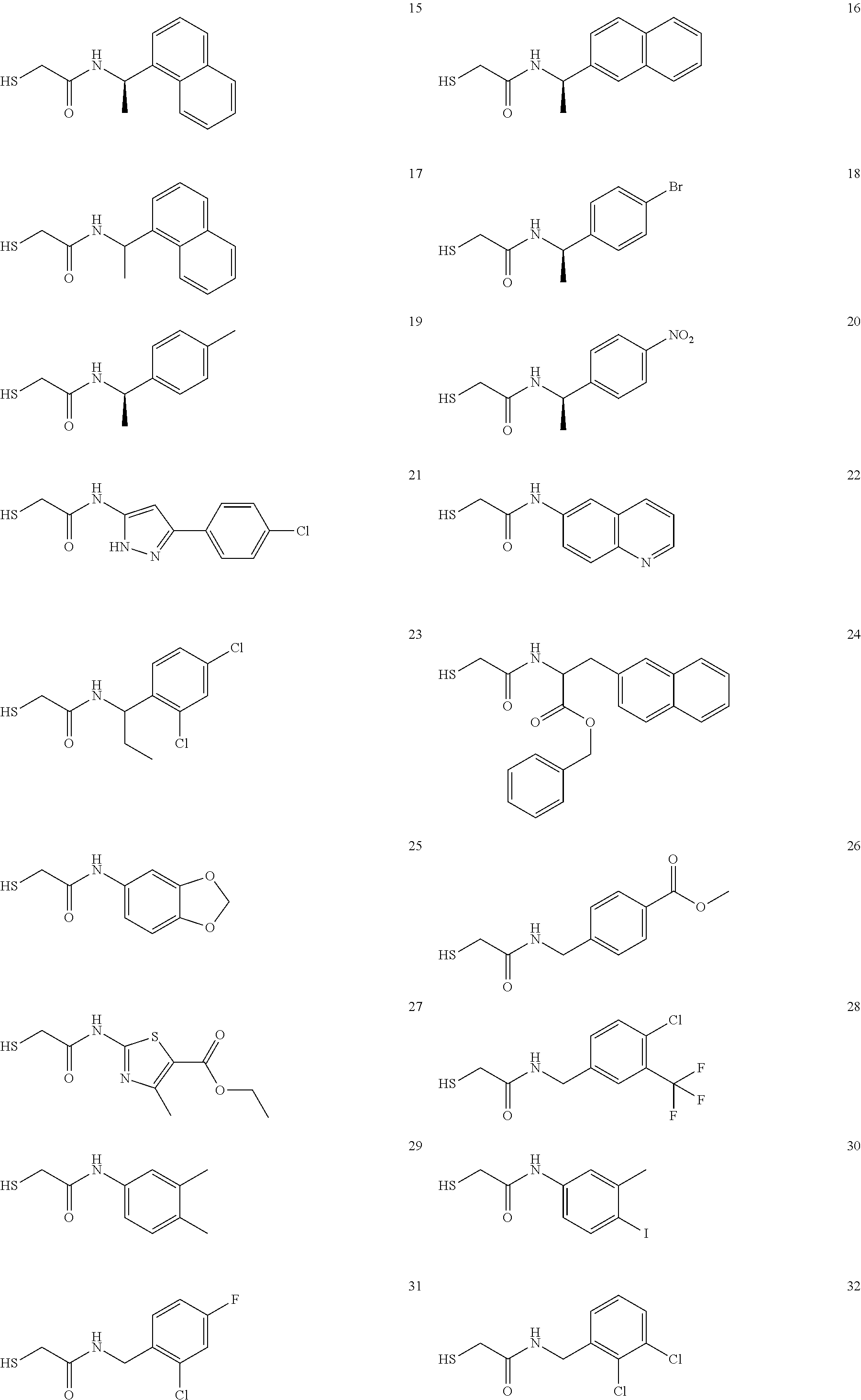

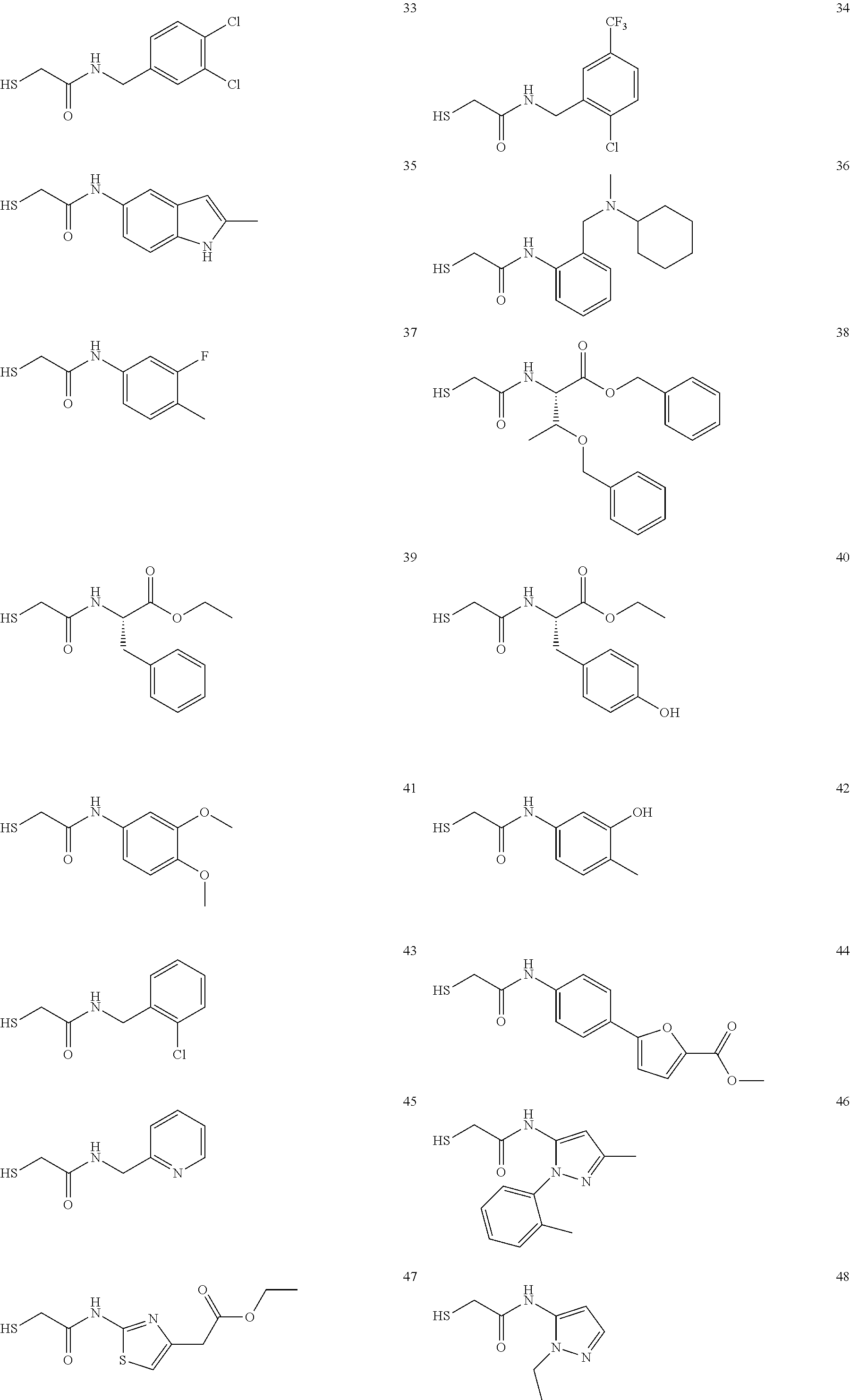

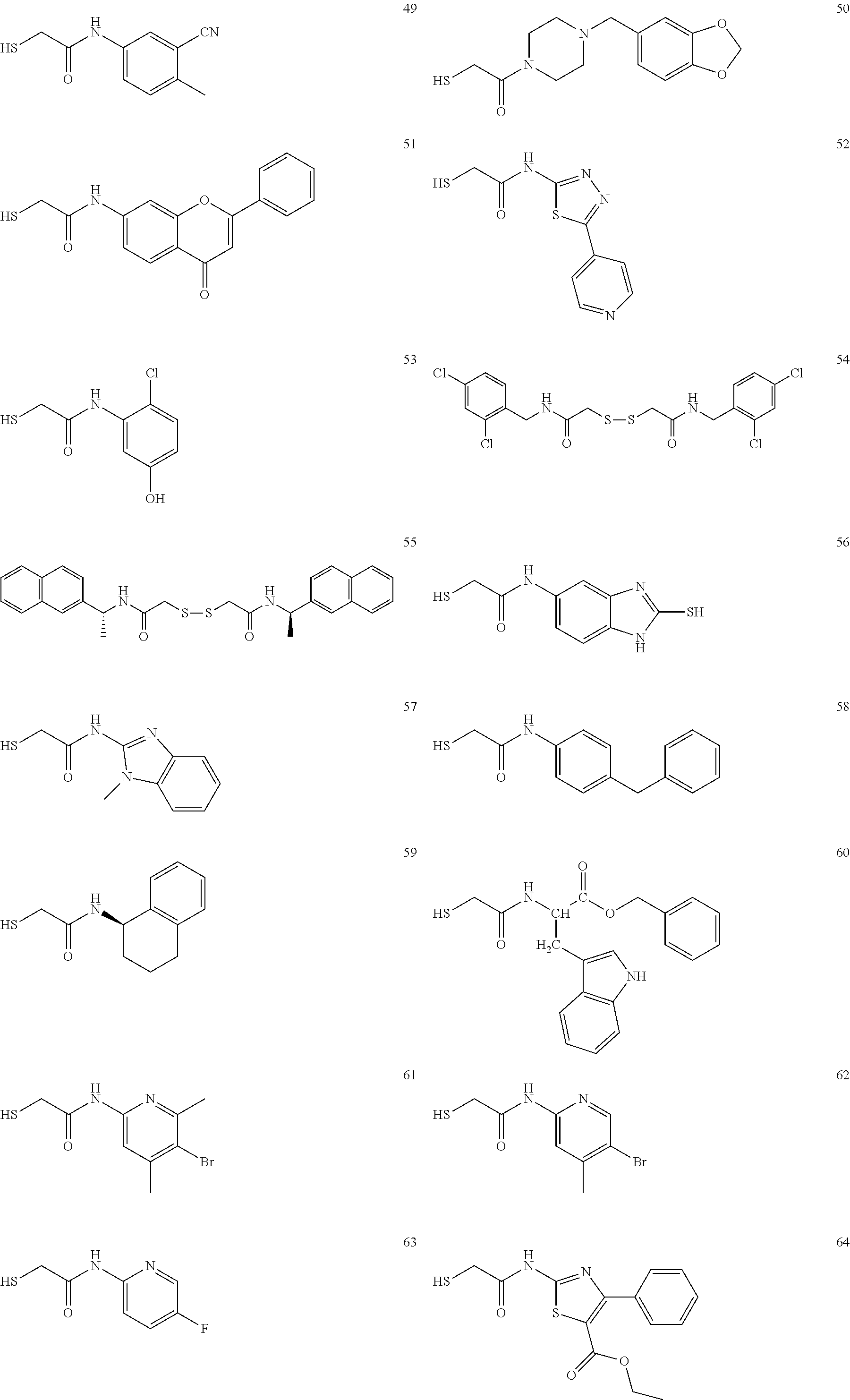

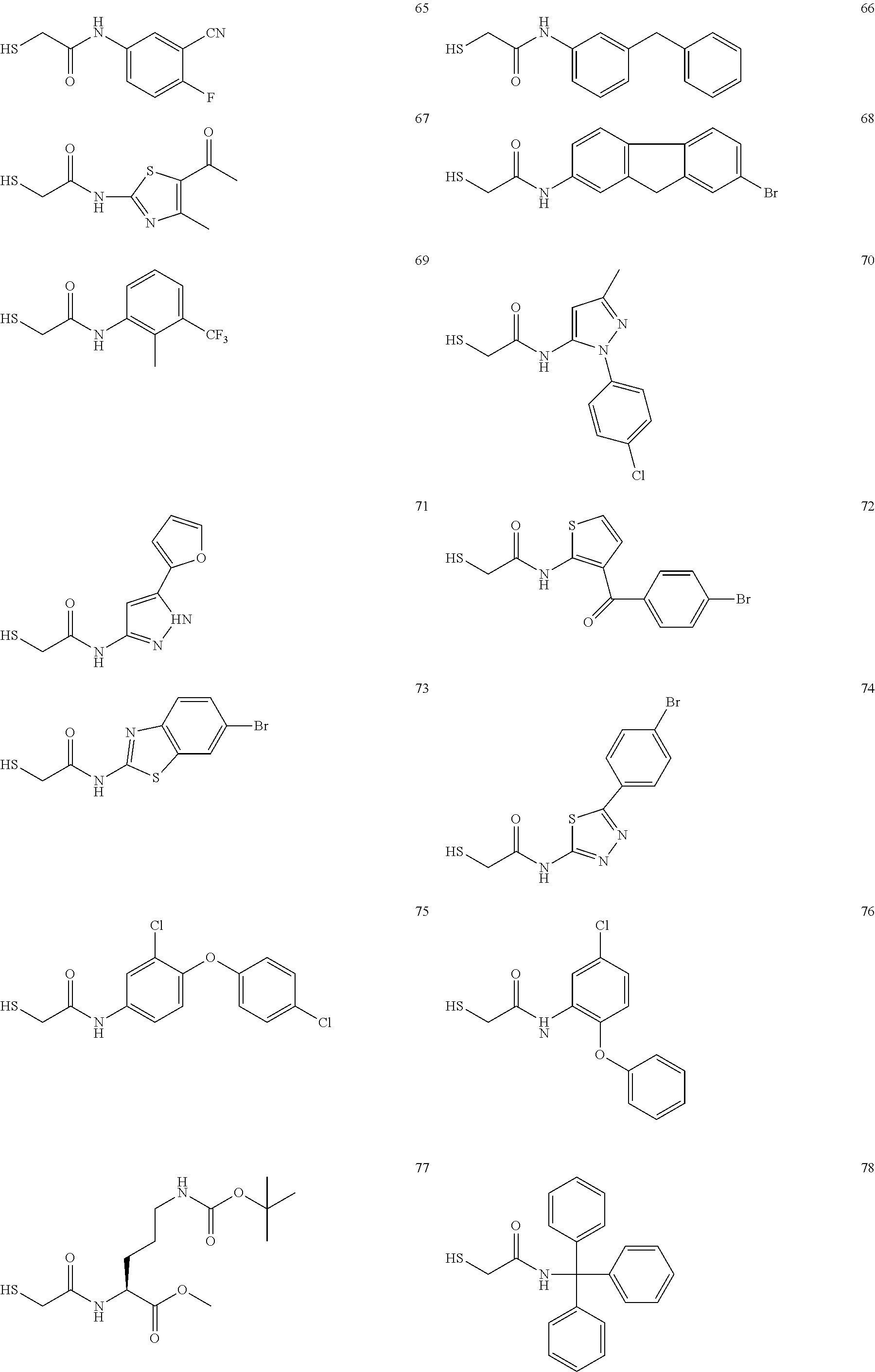

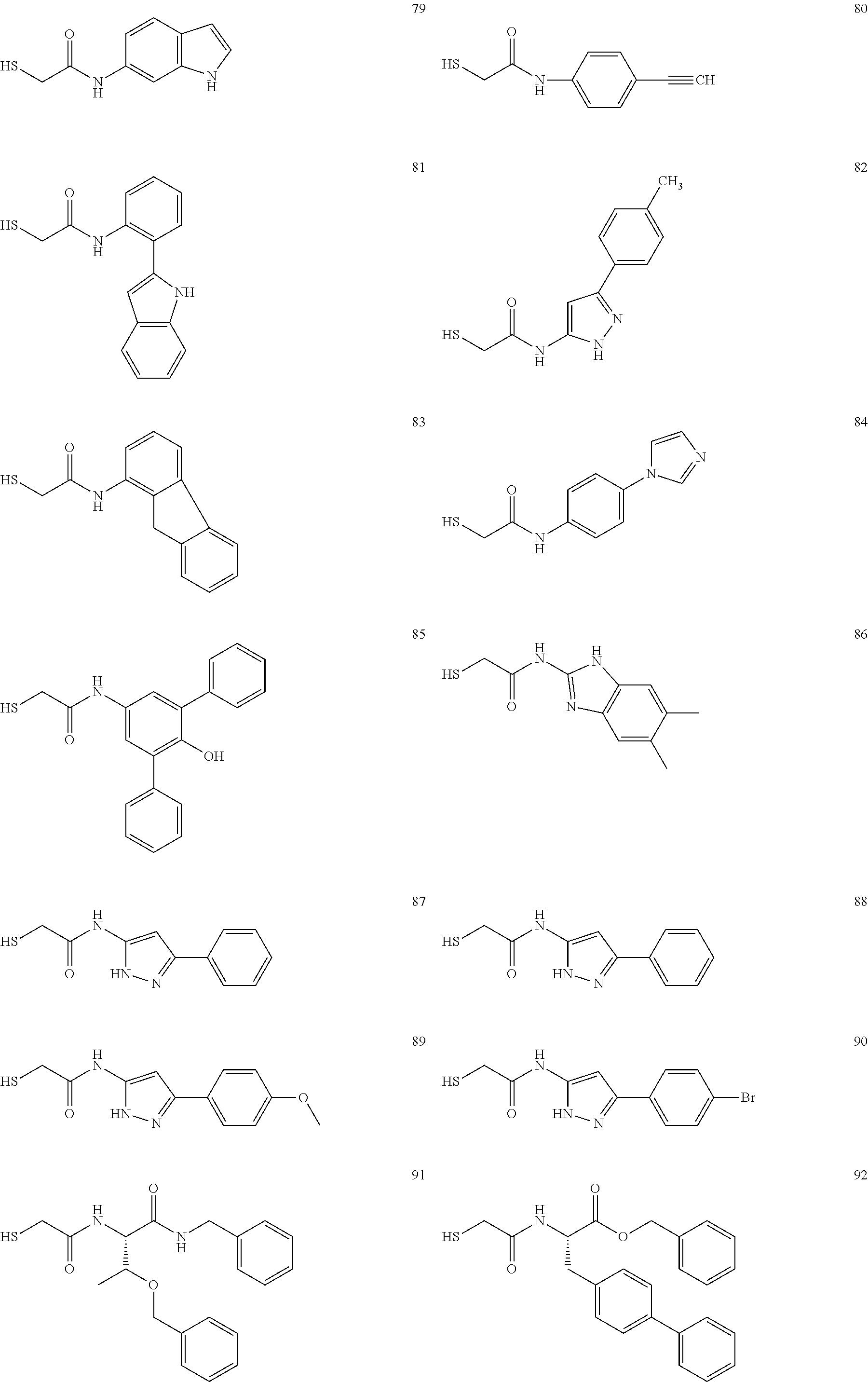

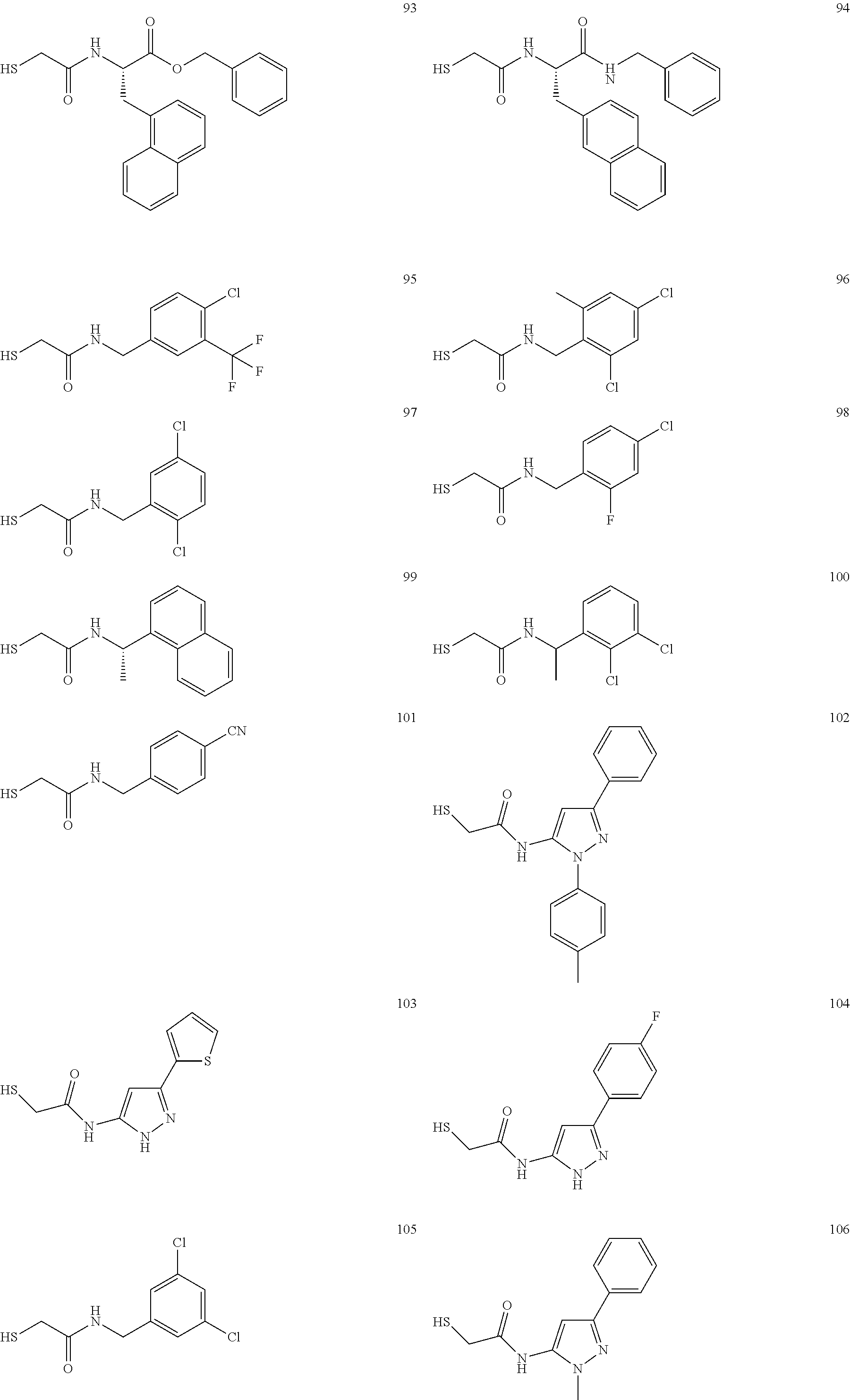

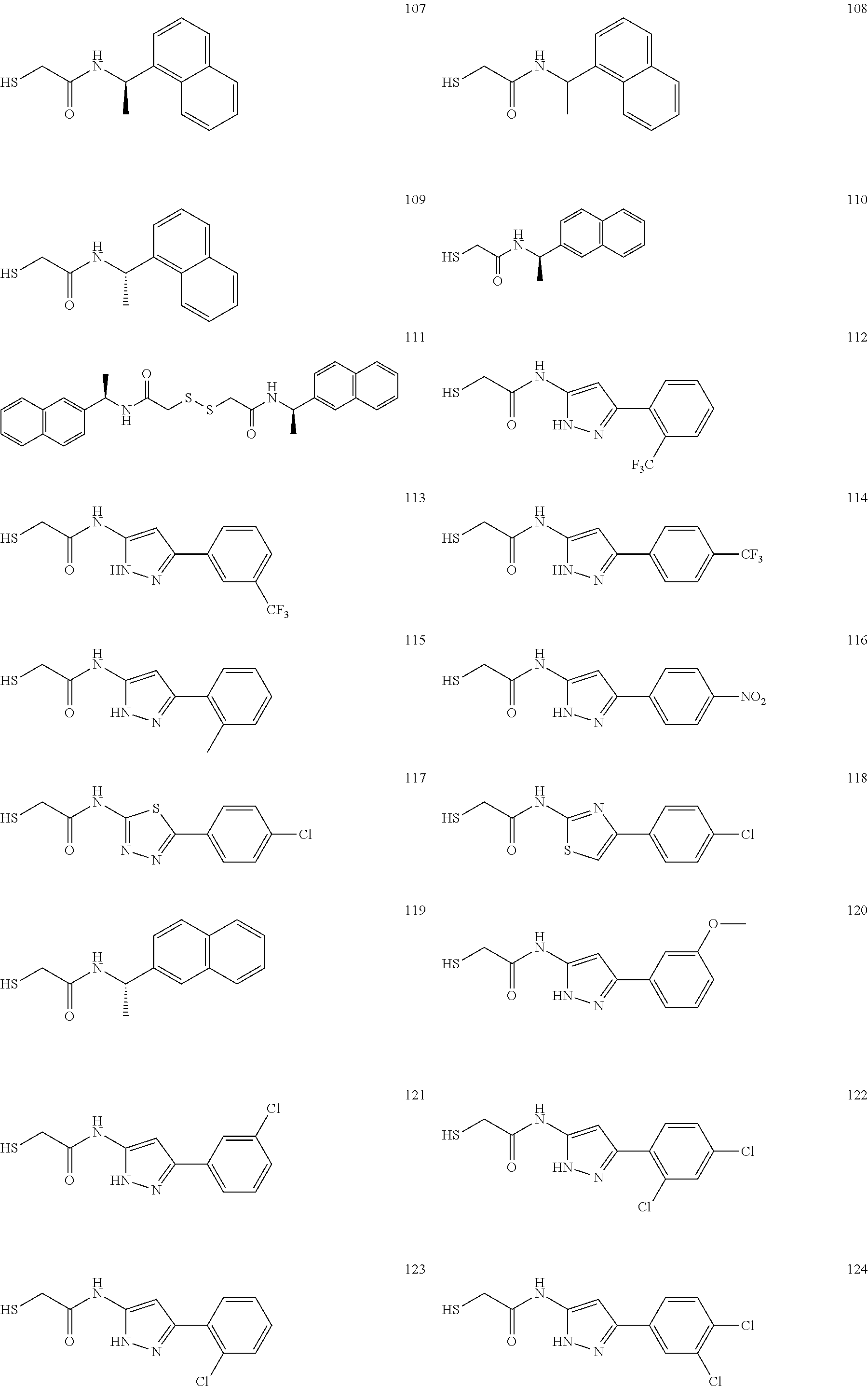

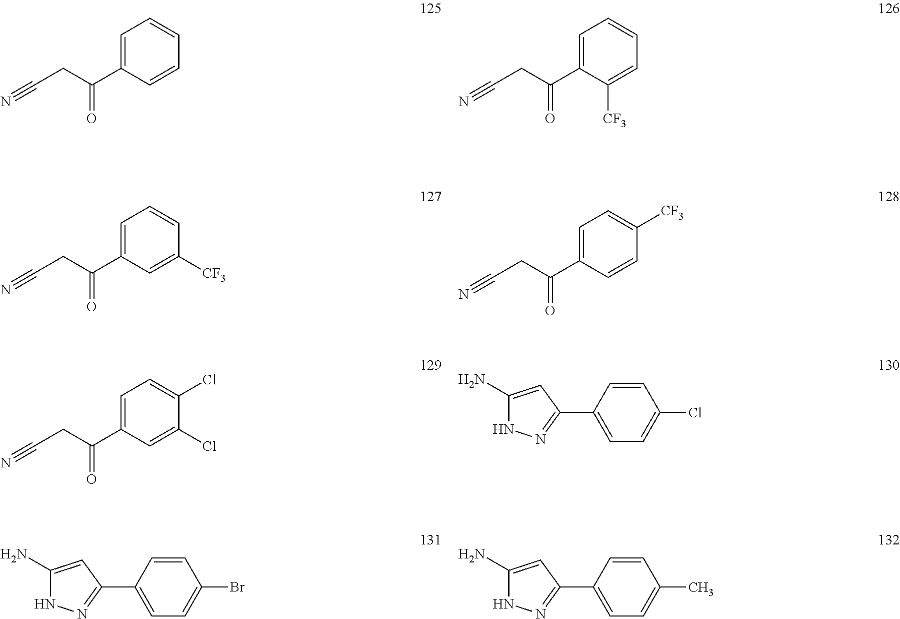

Yet another embodiment provided herein is use of a compound above in preparation of a pharmaceutical composition. Yet another embodiment is a pharmaceutical composition that includes a compound according to the above. In certain embodiments, the pharmaceutical composition has at least one of the above a compounds and an acceptable pharmaceutical carrier. Another embodiment provides use of a compound above in preparation of a pharmaceutical composition for use in treatment of a subject afflicted with a condition related to botulinum toxin.

The terms below shall have the following meanings herein and in the claims, unless otherwise required by the context.

A compound having a plurality of tautomeric forms is not limited to any one specific tautomer. The compound includes the full range of tautomeric forms of the compound. Further, as is evident to those skilled in the art, the compounds herein contain asymmetric carbon atoms. It should be understood, therefore, that the full range of stereoisomers are within the scope of this invention.

The term, “unsubstituted” refers to an atom absent a substituent at the designated atom, or that has a substituent that is a hydrogen atom.

The term, “substituted” refers to one or more hydrogen atoms covalently bonded to the designated atom is replaced by a specified group, provided that the valence on the designated atom is not exceeded, and that a chemically stable compound results from the substitution.

The term “heteroatom” refers to an oxygen, a sulfur, or a nitrogen atom substituted at a designated atom.

The term, “C₁-C₆ alkyl”, “lower alkyl” or “alkyl” refer to a straight or branched chain alkyl group having 1-6 carbon atoms, such as, for example, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. The term “higher alkyl” refers to a straight or branched chain alkyl group having 6-12 carbon atoms.

The term, “C₁-C₆ heteroalkyl” refers to a C₁-C₆ alkyl group in which one or more of the carbon atoms have been replaced with a heteroatom, for example O, N, or S.

The term, “C₂-C₆ alkenyl” refers to a hydrocarbon chain having 2 to 6 carbon atoms in a straight or a branched arrangement and containing one or more unsaturated carbon-carbon double bonds that occur between two adjacent carbon atoms at any stable point in the chain, such as, for example, ethenyl (vinyl), allyl, isopropenyl, and the like.

The term, “C₂-C₆ alkynyl” refers to a hydrocarbon chain that has 2 to 6 carbon atoms in a straight or branched arrangement and containing one or more unsaturated carbon-carbon triple bonds that occur between two carbon atoms at any stable point in the chain, such as, for example, ethynyl, propargyl, and the like.

The term, “C₃-C₆ cycloalkyl” refers to an alkyl group that has 3-6 carbon atoms that form a monocyclic ring system, such as, for example, cyclopropyl, cyclobutyl, cyclopentyl, cyclohexyl, cycloheptyl, and the like.

The term, “C₃-C₆ heterocycloalkyl” refers to a C₃-C₆ cycloalkyl group in which one or more of the ring carbon atoms have been replaced with a heteroatom, for example O, N, or S. Examples of such compounds include tetrahydropyran, tetrahydropyrrole, tetrahydrothiophene, piperidine, dioxane, dithiane, and piperazine.

The term, “C₃-C₆ cycloalkenyl” refers to an alkyl group that has 3-6 carbon atoms that form a monocyclic ring system and contain one or more carbon-carbon double bonds between two carbon atoms, preferably in a stable position, in the ring, such as, for example, cyclopentenyl, cyclohexenyl, or cycloheptenyl.

The term, “C₃-C₆ heterocycloalkenyl” refers to C₃-C₆ cycloalkenyl group in which one or more of the ring carbon atoms have been replaced with a heteroatom, for example O, N, or S. Examples of such compounds include pyrazole, pyrazoline, thiazole, thiadiazole, isothiazole, oxazole, imidazole, furan, and thiophene.

The term, “aryl” refers to a monocyclic aromatic group that has 6 to 10 carbon atoms, such as, for example, phenyl, naphthyl, indenyl, azulenyl, and anthryl.

The term, “heteroaryl” refers to an aryl group in which one or more of the ring carbon atoms have been replaced with a heteroatom, for example O, N, or S. Examples of such compounds include pyridine, pyrimidine, pyrazine, and pyridazine. It also includes fused ring systems including indole, benzimidazole, phenothiazinyl, and the like.

The term “C₁-C₆ cycloalkylaryl” refers to a cycloalkyl group that has 3-6 carbon atoms that are fused to an aryl group. Examples of such compounds include indane and tetrahydronaphthalene. The C₁-C₆ cycloalkylaryl functional group is attached to the remaining atoms in the structure at a carbon atom in the cycloalkyl group or at a carbon atom in the aryl group.

The term, “heterocycloalkylaryl” refers to a cycloalkyl group that has 3-6 carbon atoms that are fused to an aryl group in which one or more of the ring carbon atoms in the cycloalkyl group have been replaced with a heteroatom, for example O, N, or S. Examples of such compounds include isoindoline, benzodioxane, and indoline. The heterocycloalkylaryl functional group is attached to the remaining atoms in the structure at an atom in the heterocycloalkyl group or at a carbon atom in the aryl group.

The term, “C₃-C₆ cycloalkenylaryl” refers to a cycloalkenyl group having 3-6 carbon atoms that are fused to an aryl group. Examples of such compounds include indene, isoindene and naphthalene. The C₃-C₆ cycloalkenylaryl functional group is attached to the remaining atoms in the structure at a carbon atom in the cycloalkenyl group or at a carbon atom in the aryl group.

The term, “C₃-C₆ heterocycloalkenylaryl” refers to a cycloalkenyl group having 3-6 carbon atoms that are fused to an aryl group in which one or more of the ring carbon atoms in the cycloalkenyl group have been replaced with a heteroatom, for example O, N, or S. Examples of such compounds include indole, benzothiophene, benzimidazole, indazole, isoquinoline, quinoline, benzofuran, and phthalazine. The C₃-C₆ heterocycloalkenylaryl functional group is attached to the remaining atoms in the structure at an atom in the cycloalkenyl group or at a carbon atom in the aryl group.

The term “C₃-C₆ arylcycloalkylaryl” refers to a first aryl group fused to a cycloalkyl group having 3-6 carbon atoms which is fused to a second aryl group. The C₃-C₆ arylcycloalkylaryl functional group is attached to the remaining atoms in the structure at a carbon atom in the cycloalkyl group or at a carbon atom in either of the aryl groups. Examples include compounds of Formula VI:

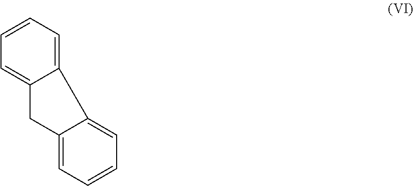

The term, “alkoxy” refers to a straight or branched chain alkoxy group having 1-6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

The term, “oxo” (indicated herein as ═O) refers to a double-bond oxygen group that is formed by replacing two geminal hydrogen atoms on a carbon atom with a double-bond oxygen group.

The term “halo” refers to any of fluoro, chloro, bromo and iodo.

The term “cyano” refers to a carbon atom joined to a nitrogen atom by a triple bond.

The term “salts” includes for example, pharmaceutically acceptable salts of a compound herein. Such salts are formed, for example, as acid addition salts, including organic or inorganic acids, from compounds herein with a basic nitrogen atom, including pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, pro-pionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids such as glutamic acid or aspartic acid, maleic acid, hydroxy-maleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, and other organic protonic acids, such as ascorbic acid.

In the presence of a negatively charged ion, such as a carboxy or a sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acidic group are present in the same molecule, a compound of the present invention may also form an internal salt, a zwitterion.

For purposes of isolation or purification salts that are not necessarily pharmaceutically acceptable, for example picrates or perchlorates, are within the scope of the invention. For therapeutic use, pharmaceutically acceptable salts or free compounds are employed (in the form of pharmaceutical preparations).

Reference to the compounds herein before and hereinafter is to be understood as referring also to the corresponding tautomers of these compounds, tautomeric mixtures of these compounds, or salts of any of these, as appropriate and expedient and if not mentioned otherwise, in view of the close relationship between the compounds in free form and those in the form of their salts, including those salts that can be used as intermediates, for example in the purification or identification of the compounds, tautomers or tautomeric mixtures and their salts.

The present invention relates also to a pro-drug of a compound provided herein, that is converted in vivo to a compound provided herein. Reference to a compound of the present invention therefore encompasses a corresponding pro-drug of the compound of the present invention, as appropriate and expedient.

The present invention relates also to active metabolites that are biologically generated after administration of one or more of the claimed analogs into a mammal. It is conceivable that the active metabolite could be isolated and identified and subsequently used as a drug itself.

The present invention relates also to a pharmaceutically acceptable substituent of a compound of the present invention. The term, “pharmaceutically acceptable substituent” refers to a structural modification that is made to a compound herein that does not materially alter the structure-activity relationship of the compound. For example, a successful bioisosteric replacement or substitution of a functional group or system in the compounds of Formulas I-V, as is well known in the art, provides a clinically useful compound (structural homolog, analog, and/or congener) with similar biopharmaceutical properties and activities against botulinum toxin. Examples of pharmaceutically acceptable substituents and methods of obtaining such compounds are found in Foye et al. (Principals of Medicinal Chemistry, 4th edition, Lea & Febiger/Williams and Wilkins, Philadelphia, Pa., 1995).

### Uses to Treat a Botulinum Toxin Related Condition

It has been found that the compounds of the present invention have valuable pharmacological properties and are useful in the treatment of botulinum toxin, for example botulinum toxin of serotype A. Examples of botulinum toxin include serotype A, serotype B, serotype C, serotype D, serotype E, serotype F, and serotype G. Botulinum toxin is characterized as a two-chain polypeptide with a 100-kDa heavy chain joined by a disulfide bond to a 50-kDa light chain. The light chain of the botulinum toxin polypeptide is a compact globule consisting of a mixture of α-helices, β-sheets, and strands with a gorge-like zinc containing a metalloprotease active site, 15-20 Å deep, depending on serotype (Boldt et al. J. Comb. Chem. 2006, 8, 513-521).

The heavy chain of the polypeptide is important for targeting the toxin to specific types of axon terminals. Following attachment of the polypeptide heavy chain to proteins on the surface of axon terminals, the toxin is taken into neurons by endocytosis. Once the toxin is within the neurons, the light chain of the toxin is able to leave endocytotic vesicles and reach the cytoplasm. The light chain is an enzyme (a zinc protease) that attacks SNARE fusion proteins (SNAP-25, syntaxin or synaptobrevin) at a neuromuscular junction. Botulinum toxin types B, D, F, and G cleave synaptobrevin, botulinum toxin types A, C, and E cleave SNAP-25, and botulinum toxin type C cleaves syntaxin (Arnon et al., JAMA. 2001; 285:1059-1070).

The SNARE proteins allow the membrane of the synaptic vesicle containing a neurotransmitter, e.g., acetylcholine, to fuse with the neuronal cell membrane. The SNARE proteins are thus important for release of neurotransmitters from axon endings (Foran et al., 2003, J. Biol. Chem. 278(2): 1363-1371). After membrane fusion, acetylcholine is released into the synaptic cleft and then bound by receptors on the muscle cell. Botulinum toxin specifically cleaves these SNAREs, and thus prevents neuro-secretory vesicles from docking/fusing with the nerve synapse plasma membrane and releasing their neurotransmitters, e.g., acetylcholine. By inhibiting acetylcholine release, the toxin interferes with nerve impulses and causes flaccid (sagging) paralysis of muscles because the muscle is unable to contract without acetylcholine.

Without being limited by any particular theory or mechanism of action, it is believed that compounds of the invention herein bind to the zinc molecule in the active site of the light chain of the botulinum toxin, thus inhibiting the ability of the botulinum toxin to cleave the SNARE proteins. By inhibiting the ability of the botulinum toxin to cleave the SNARE proteins, neuro-secretory vesicles are allowed to dock/fuse with the nerve synapse plasma membrane and release their neurotransmitters.

The phrase “treatment of botulinum toxin” refers to the prophylactic or therapeutic (including palliative and/or curing) treatment of the toxin and conditions associated with, caused by, or related to the toxin, including for example, botulism. The term “use” includes any one or more of the following embodiments of the invention, respectively: use in the treatment of botulinum intoxication and related conditions; use for the manufacture of pharmaceutical compositions for use in the treatment of botulinum intoxication and related conditions; methods of use of derivatives of Formulas I-V in the treatment of these conditions; pharmaceutical preparations having derivatives of Formulas I-V for the treatment of these conditions; and derivatives of Formulas I-V for use in the treatment of these conditions, as appropriate and expedient, if not stated otherwise.

In particular, conditions to be treated by a compound of the present invention are selected from botulinum toxin related conditions (“related” meaning also “supported” or “associated”) diseases, including those corresponding to botulism and those conditions that depend on botulinum toxin.

The term “use” further includes embodiments of compounds herein that bind to a botulinum toxin protein sufficiently to serve as tracers or labels, so that when coupled to a fluorophore or tag, or in a radioactive form, are research reagents or as diagnostics or imaging agents. Thus in another embodiment, the compounds of the present invention are also useful as probes.

Working examples provided herein demonstrate in vivo anti-botulinum toxin activity of compounds provided herein.

Embodiments of the compounds of the present invention have pharmacological properties useful in the treatment of botulinum related conditions, for example, botulism. Other embodiments of the compounds of the present invention have binding properties useful in diagnostic and labeling capacities and as imaging agents. Other embodiments of the compounds of the present invention are useful in protein purification capacities, i.e., purifying a botulinum toxin protein from a mixture of components in a sample.

### Assays

Numerous assays are known to one of ordinary skill in the art for testing compounds of the invention for inhibitory activity against botulinum toxin. Exemplary enzyme assays include those shown in the following references: Schmidt et al. J. Protein Chemistry, 14(8):703-708 1995; Schmidt et al. J. Protein Chemistry, 16(1):19-26 1997; Schmidt et al. Analytical Biochemistry, 296:130-137 2001; Schmidt et al. Applied and Environ. Microbiology, 69(1):297-303, 2003; and Boldt et al. J. Combinatorial Chemistry, 8, 513-521, 2006. Exemplary cellular assays include those shown in the following references: Zhou et al. FEBS Letters, 555:375-379, 2003; Sheridan et al. Toxicon 45, 377-382, 2005; and Pellett et al. FEBS Letters, 581:4803-4808, 2007.

### IC₅₀ Calculations

Input: 3×4 μL stopped assay on Immobilon membrane,
background (3 wells): assay with H₂O instead of enzyme;
positive control (4 wells): 3% DMSO instead of compound;
bath control (1 well): no reaction mix.

IC₅₀ values are calculated by logarithmic regression analysis of the percentage inhibition of each compound at a minimum of 4 concentrations (usually 3- or 10-fold dilution series starting at 10 μM). In each experiment, the actual inhibition by reference compound is used for normalization of IC₅₀ values to the basis of an average value of the reference inhibitor:

Normalized IC₅₀=measured IC₅₀ average ref. IC₅₀/measured ref. IC₅₀

Example: Reference inhibitor in experiment 0.4 average 0.3 μM
Test compound in experiment 1.0 μM, normalization: 0.3/0.4=0.75 μM For example, staurosporine or a synthetic staurosporine derivative are used as reference compounds.

Using this protocol, the compounds provided herein are found to have IC₅₀ values for botulinum toxin inhibition in the range from about 0.005 to about 100 μM, or about 0.002 to about 50 μM, including, for example, the range from about 0.001 to about 2 μM or lower.

### Synthetic Procedure

Compounds provided herein are prepared from commonly available starting materials using procedures known to those skilled in the art, including any one or more of the following procedures without limitation.

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known by one of ordinary skill in the art of chemistry. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralization of salts, such as acid addition salts, to the isoelectric point, e.g. with weak bases, or by treatment with ion exchangers.

Salts can be converted in a customary manner into the free compounds; metal and ammonium salts can be converted, for example, by treatment with suitable acids, and acid addition salts, for example, by treatment with a suitable basic agent.

Mixtures of isomers are obtained according to methods provided herein and are separated in a manner known to one of ordinary skill in the art of chemistry into the individual isomers; diastereoisomers are separated, for example, by partitioning between polyphasic solvent mixtures, recrystallisation and/or chromatographic separation, for example over silica gel or by e.g. medium pressure liquid chromatography over a reversed phase column, and racemates are separated, for example, by the formation of salts with optically pure salt-forming reagents and separation of the mixture of diastereoisomers so obtainable, for example by fractional crystallisation, or by chromatography over optically active column materials.

Intermediates and final products are further purified according to standard methods, e.g. using chromatographic methods, distribution methods, (re-) crystallization, and the like.

### General Process Conditions

The following applies in general to all processes mentioned herein before and hereinafter.

All the above-mentioned process steps are carried out under reaction conditions that are well known in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., including, for example, from about −80° C. to about 150° C., for example at from about −80 to about 60° C., at room temperature, at from about −20 to about 40° C. or at reflux temperature, under atmospheric pressure or in a closed vessel, where appropriate under pressure, and/or in an inert atmosphere, for example under an argon or nitrogen atmosphere.

At each stage of the reactions, mixtures of isomers that are formed can be separated into the individual isomers, for example diastereoisomers or enantiomers, or into any desired mixtures of isomers, for example racemates or mixtures of diastereoisomers.

The solvents include solvents suitable for a particular reaction that are selected among, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethylacetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in purifying or isolating the compounds herein, for example by chromatography or partitioning.

The compounds, including their salts, may also be obtained in the form of hydrates, or their crystals may, for example, include the solvent used for crystallization. Different crystalline forms may be present.

The invention encompasses also those forms of the process in which a compound obtainable as an intermediate at any stage of the process is used as starting material and the remaining process steps are carried out; or in which a starting material is formed under reaction conditions; or is used in the form of a derivative, for example, in a protected form or in the form of a salt; or a compound obtainable by the process according to the invention is produced under the process conditions and is processed further in situ.

### Pharmaceutical Compositions

A compound described above is, in certain embodiments of the invention, provided and used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

The invention provides pharmaceutical compositions comprising a compound of the present invention, and use of pharmaceutical compositions in therapeutic or prophylactic treatment, or use in a method of treatment of a botulinum toxin related condition, including, for example, botulism, and provides compounds for use and preparation of pharmaceutical preparations.

The present invention provides also pro-drugs of a compound of the present invention that are converted in vivo to the compound of the present invention. Any reference to a compound of the present invention is therefore to be understood as referring also to a corresponding pro-drug of the compound of the present invention, as appropriate and expedient.

The pharmacologically acceptable compounds of the present invention may be used, for example, for the preparation of pharmaceutical compositions that comprise an effective amount of a compound of the present invention, or a pharmaceutically acceptable salt thereof, as active ingredient together or in admixture with an amount of one or more inorganic or organic, solid or liquid, pharmaceutically acceptable carriers.

The invention relates also to a pharmaceutical composition that is suitable for administration to a warm-blooded animal, including, for example, a human (or to cells or cell lines derived from a warm-blooded animal, including for example, a human cell for the treatment or, in another aspect of the invention, prevention of (i.e. prophylaxis against) a disease that responds to inhibition of botulinum toxin, comprising an amount of a compound of the present invention or a pharmaceutically acceptable salt thereof, which is effective for this inhibition, including inhibition of activity of a botulinum toxin protein interacting with a transcriptional effector protein, together with at least one pharmaceutically acceptable carrier.

The pharmaceutical compositions according to the invention are formulated for administration, for example, by a route that is enteral, such as nasal, rectal or oral, or parenteral, such as intramuscular or intravenous, the composition formulated for administration to a warm-blooded animal (including, for example, a human), formulated in an effective dose of the pharmacologically active ingredient, alone or together with an amount of a pharmaceutically acceptable carrier. The dose of the active ingredient is formulated in an amount that is suitable for the species of warm-blooded animal, using parameters such as the body weight, the age and the individual condition, individual pharmacokinetic data, the disease to be treated and the mode of administration as is well-known to one of ordinary skill in the art of pharmacology.

The dose of a compound of the present invention or a pharmaceutically acceptable salt thereof to be administered to a warm-blooded animal, for example a human of about 70 kg body weight, is for example, from about 3 mg to about 10 g, from about 10 mg to about 1.5 g, from about 100 mg to about 1000 mg/person/day. Further, the dose is divided into 1 to 3 single doses, which may, for example, be of the same size. Usually, children receive half of an adult dose.

The pharmaceutical compositions have active ingredient, for example, from about 1% to about 95%, or from about 20% to about 90% of the full amount administered, by weight. Pharmaceutical compositions according to the invention are formulated in an amount that is in unit dose form in a container, such as in the form of an ampoule, a vial, a suppository, a dragée, a tablet or a capsule.

The pharmaceutical compositions are prepared by conventional processes herein such as dissolving, lyophilizing, mixing, granulating or confectioning processes or any combination of these processes.

The compound provided herein as the active ingredient is formulated as a solution or as a suspension, and an isotonic aqueous solution or suspension. The active ingredient in certain embodiments is formulated with a carrier, for example mannitol, prior to further processes such as lyophilization. The pharmaceutical compositions may be sterilized and/or may comprise excipients, for example preservatives, stabilizers, wetting and/or emulsifying agents, solubilizers, salts for regulating the osmotic pressure and/or buffers, and are further prepared in a manner well-known in the pharmaceutical arts, such as conventional dissolving or lyophilizing processes. The solution or suspension may include a viscosity-increasing substance, such as sodium carboxymethylcellulose or carboxymethylcellulose in another form, dextran, polyvinylpyrrolidone or gelatin.

Suspensions of a compound herein formulated in oil comprise as the oil component a vegetable, synthetic or semi-synthetic oil customary for injection purposes. Oils include without limitation, liquid fatty acid esters that contain as the acid component a long-chained fatty acid having from 8 to 22, or from 12 to 22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, and further mixed if desired by addition of one or more antioxidants, for example vitamin E, β-carotene or 3,5-di-tert-butyl-4-hydroxytoluene. The alcohol component of the fatty acid ester has a maximum of 6 carbon atoms and is a mono- or poly-hydroxy, for example a mono-, di- or tri-hydroxy, alcohol, for example methanol, ethanol, propanol, butanol or pentanol or the isomers thereof, glycol and glycerol. The following are examples of fatty acid esters: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C8 to C12, Hüls AG, Germany), and vegetable oils, such as cottonseed oil, almond oil, olive oil, castor oil, sesame oil, soybean oil and groundnut oil.

The injection compositions are prepared in customary manner under sterile conditions, and are introduced into ampoules or vials and sealed into containers under sterile conditions.

Pharmaceutical compositions for oral administration are in certain embodiments obtained by combining the active ingredient with solid carriers, if desired granulating a resulting mixture, and processing the mixture, if desired or necessary, after the addition of appropriate excipients, into tablets, dragée cores or capsules. Alternatively the composition is incorporated into plastics carriers that allow the active ingredients to diffuse or be released in measured amounts.

Suitable carriers are for example, fillers such as sugars, for example lactose, saccharose, mannitol or sorbitol, cellulose preparations and/or calcium phosphates, for example tricalcium phosphate or calcium hydrogen phosphate, and binders such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators such as the above-mentioned starches, and/or carboxymethyl starch, crosslinked polyvinylpyrrolidone, agar, alginic acid or a salt thereof such as sodium alginate. Excipients are flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alfa, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations such as ethylcellulose phthalate or hydroxypropylmethylcellulose phthalate. Capsules include dry-filled capsules made of gelatin and soft sealed capsules made of gelatin and a plasticizer, such as glycerol or sorbitol. The dry-filled capsules may comprise the active ingredient in the form of granules, for example with fillers such as lactose; binders such as starches, and/or glidants such as talc or magnesium stearate, and if desired with stabilizers. In soft capsules the active ingredient is preferably dissolved or suspended in suitable oily excipients such as fatty oils, paraffin oil or liquid polyethylene glycols, and stabilizers and/or antibacterial agents can be added. Dyes or pigments may be added to the tablets or dragée coatings or the capsule casings, for example for identification purposes or to indicate different doses of active ingredient.

References and citations to other documents, such as patents, patent applications, patent publications, journals, books, papers, web contents, have been made throughout this disclosure. All such documents are hereby incorporated herein by reference in their entirety for all purposes.

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance which can be adapted to the practice of this invention in its various embodiments and equivalents thereof.

### EXAMPLES

### Example 1

### General Methods

Starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds provided herein are produced by organic synthesis methods known to one of ordinary skill in the art as illustrated in the following Examples.

Starting materials to synthesize the compounds of Formulas I-V are commercially available from, for example, Sigma-Aldrich (Millwaukee, Wis.). Table 2 below provides exemplary starting materials that were used to synthesize the compounds of Formulas I to V, and the commercial supplier of these materials.

Reactions were monitored by TLC (Silica Gel 60 F₂₅₄, EMD Chemicals) or HPLC (HP 1090). Compounds of Formulas I-V and their intermediates were purified by crystallization or silica gel flash chromatography. Characterization of compounds and intermediates were done with nuclear magnetic resonance spectroscopy (NMR) and mass spectrometry (MS).

TABLE 2
Exemplary starting materials for synthesis of the compounds of
Formulas I to V and the commercial supplier of these compounds

| Name | Supplier |
|---|---|
| 2-Amino-6-methylbenzonitrile | fluka |
| 2,5-Dimethoxyaniline | aldrich |
| 5-Aminoindan | aldrich |
| 2-Amino-3-benzyloxypyridine | aldrich |
| 2-Amino-6-methoxybenzothiazole | aldrich |
| 4-Aminobenzyl alcohol | aldrich |
| Dimethyl aminoterephthalate | aldrich |
| 3-Amino-5-phenylpyrazole | aldrich |
| 4-Amino-5-chloro-2,6-dimethylpyrimidine | aldrich |
| 5-Amino-1-phenylpyrazole-4-carbonitrile | aldrich |
| 2-Aminoacridone | fluka |
| tert-Butyl 3-aminobenzoate | fluka |
| Butyl 4-aminobenzoate | fluka |
| tert-Butyl 4-aminobenzoate | fluka |
| Methyl 3-aminobenzoate | fluka |
| Methyl 2-amino-4-chlorobenzoate | fluka |
| Methyl 2-amino-4,5-dimethoxybenzoate | fluka |
| N-Ethyl-N-isopropyl-p-phenylenediamine hydrochloride | fluka |
| 2-Amino-8-quinolinol | fluka |
| Methyl 3-amino-4-methylbenzoate | fluka |
| 4′-Aminoacetanilide | aldrich |
| 3-Benzyloxyaniline | aldrich |
| 4-Bromoaniline | aldrich |
| 2,5-Dichloroaniline | aldrich |
| 2-Methoxy-5-methylaniline | aldrich |
| 2-Amino-2′,5-dichlorobenzophenone | aldrich |
| 3-AMINO-9-ETHYLCARBAZOLE | sial |
| 2-Aminobenzothiazole | aldrich |
| 2,4-Dichloroaniline | aldrich |
| Ethyl 4-aminobenzoate | aldrich |
| 4-(Benzyloxy)aniline hydrochloride | aldrich |
| 4-Amino-1-naphthalenecarbonitrile | aldrich |
| 2-Amino-3-hydroxypyridine | aldrich |
| 2-Aminobenzyl alcohol | aldrich |
| 2-Amino-5-bromopyridine | aldrich |
| 2-Amino-4-chloro-6-methylpyrimidine | aldrich |
| 4-Amino-9-fluorenone | aldrich |
| Luminol | aldrich |
| 3,4-Dimethylaniline | aldrich |
| 5-Amino-1,3,4-thiadiazole-2-thiol | aldrich |
| Sulfanilamide | acros |
| 2-Amino-4-chlorobenzothiazole | aldrich |
| 2-Amino-3,5-dichloropyridine | aldrich |
| 2-Amino-6-chlorobenzothiazole | aldrich |
| 5-Aminoisoquinoline | aldrich |
| 2-Amino-4-methoxybenzothiazole | aldrich |
| 3′-Aminoacetophenone | aldrich |
| 3-Amino-1,2,4-triazole-5-thiol | aldrich |
| 3-Amino-2,6-dimethoxypyridine monohydrochloride | aldrich |
| 2-Amino-5-chlorothiazole hydrochloride | aldrich |
| 4-Aminobenzonitrile | aldrich |
| 6-Chloro-m-anisidine hydrochloride | aldrich |
| 4-Bromo-2-chloroaniline | aldrich |
| 4-Bromo-2-methylaniline | aldrich |
| 4-Bromo-3-methylaniline | aldrich |
| N-Boc-p-phenylenediamine | fluka |
| N1-(6-Indazolyl)sulfanilamide | aldrich |
| 5-Amino-3-methyl-isothiazole hydrochloride | aldrich |
| Diethyl 5-amino-3-methyl-2,4-thiophene dicarboxylate | aldrich |
| Ethyl 2-amino-4,5,6,7-tetrahydrobenzo[b]thiophene-3-carboxylate | aldrich |
| 3-Aminobenzonitrile | aldrich |
| 2-Aminobenzimidazole | aldrich |
| 2-Amino-5-chlorobenzonitrile | aldrich |
| 2-Amino-4,5-dimethylthiazole hydrochloride | aldrich |
| 1-Aminoisoquinoline | aldrich |
| 5-Amino-2-chloropyridine | aldrich |
| 2-Benzyloxyaniline | aldrich |
| 3-Amino-9-fluorenone | aldrich |
| 3-Amino-5-methylthio-1H-1,2,4-triazole | aldrich |
| 3-Aminobenzyl alcohol | aldrich |
| 4-Bromo-2,6-dimethylaniline | aldrich |
| 2-Amino-4-methylbenzothiazole | aldrich |
| N,N-Dimethyl-p-phenylenediamine | aldrich |
| 2-Amino-5-ethyl-1,3,4-thiadiazole | aldrich |
| 3-Methoxy-5-(trifluoromethyl)aniline | aldrich |
| 1-(2-Aminophenyl)pyrrole | aldrich |
| 2-Amino-5-trifluoromethyl-1,3,4-thiadiazole | aldrich |
| 4-Morpholinoaniline | aldrich |
| Fast Red ITR | aldrich |
| Fast Blue RR | aldrich |
| 2-Chloro-5-methylaniline | aldrich |
| 3-Aminobenzophenone | aldrich |
| 4-Amino-2-chlorobenzonitrile | aldrich |
| 2-Amino-5-bromopyrimidine | aldrich |
| 2-Amino-5-(ethylthio)-1,3,4-thiadiazole | aldrich |
| 2-Amino-5-bromobenzonitrile | acros |
| Ethyl 5-amino-1-phenyl-4-pyrazolecarboxylate | aldrich |
| 2-Amino-5-methylthiazole | aldrich |
| 2-Amino-4-methoxy-6-methylpyrimidine | aldrich |
| 5-Phenyl-10,11-dihydro-5h-dibenzo(a,d)cyclohepten-5-ylamine | salor |
| 5-Phenyl-o-anisidine | aldrich |
| 2-Amino-4-methylbenzonitrile | aldrich |
| 5-Amino-4,6-dichloropyrimidine | aldrich |
| N,N-Dimethyl-1,3-phenylenediamine dihydrochloride | aldrich |
| Ethyl 2-aminothiazole-4-acetate | aldrich |
| N-(4-Aminobenzoyl)-L-glutamic acid diethyl ester | aldrich |
| 3-Fluoro-4-methylaniline | aldrich |
| 2-(Phenylsulfonyl)aniline | aldrich |
| 3-Amino-2-methylphenol | aldrich |
| 5-Amino-2-methylphenol | aldrich |
| 3-Chloro-4-fluoroaniline | aldrich |
| 3-Chloro-4-methoxyaniline | aldrich |
| 3-Aminoquinoline | aldrich |
| Methyl 3-amino-2-thiophenecarboxylate | aldrich |
| 5-Chloro-2-methoxyaniline | aldrich |
| 2-Benzylaniline | aldrich |
| 2-Aminobenzenesulfonamide | aldrich |
| 4-Amino-N-methylphthalimide | aldrich |
| 2-Amino-4-methylbenzophenone | aldrich |
| 7-Amino-4-(trifluoromethyl)coumarin | aldrich |
| 8-Aminoquinoline | aldrich |
| 2-(4-Aminophenyl)ethanol | aldrich |
| 5-Amino-2-methoxyphenol | aldrich |
| 3,5-Difluoroaniline | aldrich |
| 3-Amino-2-methoxydibenzofuran | aldrich |
| 4-Amino-2,6-diphenylphenol | aldrich |
| 6-Aminoquinoline | aldrich |
| Methyl 3-amino-2-pyrazinecarboxylate | aldrich |
| Methyl 4-aminobenzoate | aldrich |
| Methyl 3-amino-5,6-dichloro-2-pyrazinecarboxylate | aldrich |
| Ethyl 2-amino-alpha-(methoxyimino)-4-thiazoleacetate,predominantly syn | aldrich |
| 5-Amino-3,4-dimethylisoxazole | aldrich |
| 2-Amino-6-methylbenzothiazole | aldrich |
| 5-Amino-1-ethylpyrazole | aldrich |
| 4-Bromo-3-(trifluoromethyl)aniline | aldrich |
| 1-Aminofluorene | aldrich |
| 2-Amino-3-bromo-9-fluorenone | aldrich |
| 2-Amino-7-bromofluorene | aldrich |
| 2-Amino-7-bromo-9-fluorenone | aldrich |
| 6-Amino-3,4-benzocoumarin | aldrich |
| 4-Bromo-2-fluoroaniline | aldrich |
| Methyl 2-amino-5-chlorobenzoate | aldrich |
| 5-Amino-8-hydroxyquinoline dihydrochloride | aldrich |
| 2-Amino-6-fluorobenzothiazole | aldrich |
| Methyl 4-amino-3-chlorobenzoate | acros |
| 4-Bromo-2,6-difluoroaniline | aldrich |
| p-Toluidine hydrochloride | aldrich |
| 2-Amino-5-methylbenzyl alcohol | aldrich |
| 2-Amino-3-methylbenzyl alcohol | aldrich |
| 3-Amino-2-methylbenzyl alcohol | aldrich |
| 3-Fluoro-p-anisidine | aldrich |
| 3-Amino-4-methylbenzyl alcohol | aldrich |
| 5-Methoxy-2-methylaniline | aldrich |
| 2-Methoxy-5-(trifluoromethyl)aniline | aldrich |
| 3-Phenoxyaniline | aldrich |
| 2-Amino-4-(4-chlorophenyl)thiazole | aldrich |
| 2-Amino-5-chlorobenzyl alcohol | aldrich |
| 2-Phenoxyaniline | aldrich |
| N-[4-(4-Aminobenzyl)phenyl]-5-norbornene-2,3-dicarboximide | aldrich |
| 2-Aminobenzyl cyanide | aldrich |
| 4-Amino-3-bromobenzonitrile | acros |
| 2-Amino-4,5-dimethoxybenzonitrile | aldrich |
| 2-(3-Aminophenylsulfonyl)ethanol hydrochloride | sial |
| Carbostyril 124 | aldrich |
| 2-Amino-3-chloro-5-(trifluoromethyl)pyridine | aldrich |
| 2-Amino-4,6-dimethoxypyrimidine | aldrich |
| 4-Amino-2,6-dimethoxypyrimidine | aldrich |
| 2-Methyl-3-(trifluoromethyl)aniline | aldrich |
| 5-Amino-1-naphthol | aldrich |
| 2-(2-Aminophenyl)indole | aldrich |
| 2-Amino-5-phenyl-[1,3,4]-thiadiazole | aldrich |
| 2-Amino-5-(methylthio)-1,3,4-thiadiazole | aldrich |
| 2-Amino-4-methoxy-6-methyl-1,3,5-triazine | aldrich |
| 2-Amino-4-(4-bromophenyl)thiazole | aldrich |
| 4-(1-Hydroxyethyl)aniline | aldrich |
| 3-Amino-4-chlorophenol | aldrich |
| Methyl 2-aminothiophene-3-carboxylate | aldrich |
| C,C,C-triphenyl-methylamine | salor |
| C,C,C-tri-p-tolyl-methylamine | salor |
| Pyridoxamine dihydrochloride | sigma |
| N4,N4-Diethyl-2-methyl-1,4-phenylenediamine monohydrochloride | aldrich |
| 2-Amino-5-chloro-2′-fluorobenzophenone | aldrich |
| 2-Amino-7-bromo-5-oxo-5H-[1]benzopyrano[2,3-b]pyridine-3-carbonitrile | aldrich |
| 2-Amino-4-chlorobenzonitrile | aldrich |
| 2-Amino-1-methylbenzimidazole | aldrich |
| 5-tert-Butyl-o-anisidine | aldrich |
| 2-Amino-6-(methylsulfonyl)benzothiazole | aldrich |
| 2-Amino-6-fluorobenzonitrile | aldrich |
| 4-Methoxy-3-biphenylamine hydrochloride | aldrich |
| 3,4-Dichloroaniline | aldrich |
| 2,5-Diethoxy-4-morpholinoaniline dihydrochloride | aldrich |
| 4-(1H-Imidazol-1-yl)aniline | aldrich |
| 2-Amino-4,5-dimethyl-3-furancarbonitrile | aldrich |
| 2-Amino-N-cyclohexyl-N-methylbenzylamine | aldrich |
| 2-Amino-5-phenyl-1,3,4-thiadiazole sulfate salt | aldrich |
| 4-Chloroaniline | aldrich |
| 4-Amino-5-pyrimidinecarbonitrile | aldrich |
| 4-Ethynylaniline | aldrich |
| 4-(Trifluoromethylsulfonyl)aniline | aldrich |
| 3′-Aminoacetanilide | aldrich |
| 4-Amino-6-methoxypyrimidine | aldrich |
| 3′-Amino-4′-chloroacetanilide | aldrich |
| 2-Amino-4-chloro-6-methoxypyrimidine | aldrich |
| 4-Amino-2-chloro-6,7-dimethoxyquinazoline | aldrich |
| 2-Amino-5-fluoropyridine | aldrich |
| 4-Bromo-3-fluoroaniline | aldrich |
| 4-Amino-3-chlorobenzonitrile | aldrich |
| 2-Amino-6-bromopyridine | aldrich |
| 2-Amino-N-cyclohexyl-N-methylbenzenesulfonamide | aldrich |
| 4-Amino-2-chloropyridine | aldrich |
| 2,5-Diethoxyaniline | aldrich |
| 2-Amino-5-tert-butyl-1,3,4-thiadiazole | aldrich |
| 2-Amino-5-bromo-3-methylpyridine | aldrich |
| 5-Amino-2-methylindole | aldrich |
| 4-[(N-Boc)aminomethyl]aniline | aldrich |
| 8-Amino-6-methoxyquinoline hydrobromide | aldrich |
| Methyl 2-amino-5-bromobenzoate | aldrich |
| 4-Chloro-3-methylaniline | aldrich |
| 2-Bromo-5-fluoroaniline | aldrich |
| 5-Amino-1,3-dimethylpyrazole | aldrich |
| 2-Amino-4-(p-tolyl)-thiazole | aldrich |
| 2-Amino-5,6-dimethyl-4-hydroxypyrimidine | aldrich |
| 3-Amino-5-(4-methoxyphenyl)pyrazole | aldrich |
| 5-Amino-2-mercaptobenzimidazole | aldrich |
| 4-Chloro-2,5-dimethoxyaniline | aldrich |
| 2-Amino-6-bromobenzothiazole | aldrich |
| 5-Aminopyridine-2-carbonitrile | aldrich |
| Phenyl-2-aminobenzenesulfonate | aldrich |
| 5-Amino-3-methyl-1-phenylpyrazole | aldrich |
| 2-Amino-3-bromo-5-methylpyridine | aldrich |
| 2-Bromo-4-chloroaniline | aldrich |
| 2-Amino-4-hydroxy-6-trifluoromethylpyrimidine | aldrich |
| Methyl 3-amino-4-methylthiophene-2-carboxylate | aldrich |
| N-(3-Aminophenyl)methanesulfonamide | aldrich |
| 6-Amino-3-bromo-2-methylpyridine | aldrich |
| 5-Amino-3-(4-methylphenyl)-1-phenylpyrazole | aldrich |
| 5-Amino-3-(4-methoxyphenyl)-1-phenylpyrazole | aldrich |
| 5-Amino-3-(4-methylphenyl)pyrazole | aldrich |
| 5-Amino-2-methylbenzonitrile | aldrich |
| 2′-Aminoacetanilide | aldrich |
| 3-Amino-6-bromopyridine | aldrich |
| N-(3-Aminophenyl)propanamide | aldrich |
| 4-Fluoro-3-methylaniline | aldrich |
| 3,4,5-Trifluoroaniline | aldrich |
| 4-Benzylaniline | aldrich |
| 3-Benzylaniline | aldrich |
| 3-Chloro-4-(4-chlorophenoxy)aniline | aldrich |
| 6-Aminobenzothiazole | aldrich |
| 2-Amino-4-chlorophenyl phenyl ether | aldrich |
| 2-Amino-N-ethyl-N-phenylbenzenesulfonamide | aldrich |
| 2-Amino-5-(4-bromophenyl)-1,3,4-thiadiazole | aldrich |
| 3-Amino-5-(2-furyl)pyrazole | aldrich |
| 5-Amino-2-fluorobenzonitrile | aldrich |
| 5-Amino-3-(4-chlorophenyl)isoxazole | aldrich |
| 2-Amino-3-(4-bromobenzoyl)thiophene | aldrich |
| 4-Amino-3-(trifluoromethyl)pyridine | aldrich |
| 4′-Aminoacetophenone | aldrich |
| 2-Phenylglycinonitrile hydrochloride | aldrich |
| 4-Hydroxy-3-methoxybenzylamine hydrochloride | aldrich |
| 4-Chlorobenzhydrylamine hydrochloride | aldrich |
| 4-Benzyloxy-3-chloroaniline | aldrich |
| 4-Amino-2-bromopyrimidine-5-carbonitrile | aldrich |
| 4-Amino-2-(1-piperidinyl)pyrimidine-5-carbonitrile | aldrich |
| 4-Amino-2,6-dichloropyridine | aldrich |
| 4-Bromo-3-methyl-1-phenyl-1H-pyrazol-5-ylamine | aldrich |
| 3-Methyl-1-(2-methylphenyl)-1H-pyrazol-5-amine | aldrich |
| 1-(4-Chlorophenyl)-3-methyl-1H-pyrazol-5-ylamine | aldrich |
| 2-Amino-5-(4-chlorophenyl)-[1,3,4]-thiadiazole | aldrich |
| 5-Amino-1H-[1,2,4]-triazole-3-carboxylic acid methyl ester | aldrich |
| 2-Amino-5-(4-methoxyphenyl)-1,3,4-thiadiazole | aldrich |
| 6-Aminoflavone | aldrich |
| 7-Aminoflavone | aldrich |
| Ethyl 4-aminocinnamate | aldrich |
| 4-(Heptyloxy)aniline | aldrich |
| 2-Amino-5-bromo-4-methylpyridine | aldrich |
| 7-Amino-2-methylchromone | aldrich |
| 5-Amino-2-(trifluoromethyl)benzimidazole | aldrich |
| 5-Acetyl-2-amino-4-methylthiazole | aldrich |
| 5-Amino-3-(2-thienyl)pyrazole | aldrich |
| 5-Amino-2-chlorobenzyl alcohol | aldrich |
| 4-Amino-2-chloropyrimidine-5-carbonitrile | aldrich |
| 1,10-Phenanthrolin-5-amine | aldrich |
| 4-Amino-2-(trifluoromethyl)benzonitrile | aldrich |
| 2-Acetamido-5-aminopyridine | aldrich |
| 2-Amino-5-bromo-4,6-dimethylpyridine | aldrich |
| 2-Amino-5-bromopyrazine | aldrich |
| 6-Amino-3-pyridinecarbonitrile | aldrich |
| 4′-Aminobenzanilide | aldrich |
| 5-Bromo-4-fluoro-2-methylaniline | aldrich |
| 2-Amino-3-pyridinecarboxaldehyde | aldrich |
| 5-Amino-2-(trifluoromethyl)pyridine | aldrich |
| N-(2-Amino-4,5-dichlorophenyl)acetamide | aldrich |
| Ethyl 3-aminobenzofuran-2-carboxylate | aldrich |
| 2-(2-Aminophenyl)benzothiazole | aldrich |
| 2-Amino-4-(3,4-difluorophenyl)thiazole | aldrich |
| 4-(3-Aminophenyl)-2-methylthiazole | aldrich |
| 2-Amino-4-phenylthiazole | aldrich |
| 2-Amino-5-fluorobenzonitrile | aldrich |
| 3-(4-Aminophenyl)benzonitrile | aldrich |
| 4-(3-Aminophenyl)benzonitrile | aldrich |
| 5-Amino-3-(4-methoxyphenyl)isoxazole | aldrich |
| 5-Amino-3-phenylisoxazole | aldrich |
| Ethyl 2-amino-4-methylthiazole-5-carboxylate | aldrich |
| 5-Amino-3-(4-chlorophenyl)pyrazole | aldrich |
| 5-Amino-3-(4-fluorophenyl)pyrazole | aldrich |
| 5-Amino-3-(4-bromophenyl)pyrazole | aldrich |
| Methyl 5-(3-aminophenyl)furan-2-carboxylate | aldrich |
| Methyl 5-(4-aminophenyl)furan-2-carboxylate | aldrich |
| Methyl 6-aminonicotinate | aldrich |
| Ethyl 3-amino-5-(4-chlorophenyl)thiophene-2-carboxylate | aldrich |
| 4-(Thiophen-3-yl)aniline | aldrich |
| 2-Amino-5-chloropyrimidine | aldrich |
| Ethyl 2-amino-4-phenylthiazole-5-carboxylate | aldrich |
| 3-Amino-4-chlorobenzonitrile | aldrich |
| Methyl 4-amino-3-bromobenzoate | aldrich |
| 2-Amino-4,6-dichloropyrimidine-5-carboxaldehyde | aldrich |
| 2-Amino-5-methyl-4-phenylthiazole | aldrich |
| 5-Amino-1-(3-chlorophenyl)-1H-pyrazole-4-carbonitrile | aldrich |
| 5-Amino-1-(4-chlorophenyl)-1H-pyrazole-4-carbonitrile | aldrich |
| 2-Amino-6-(trifluoromethyl)pyridine | aldrich |
| 3-Amino-2-bromopyridine | aldrich |
| Ethyl 2-amino-4-methylpyrimidine-5-carboxylate | aldrich |
| 2-Amino-5-(4-methoxyphenyl)-1,3,4-oxadiazole | aldrich |
| 2-Amino-5-(4-chlorophenyl)-1,3,4-oxadiazole | aldrich |
| 2-Amino-5-(2-chlorophenyl)-1,3,4-oxadiazole | aldrich |
| 2-Amino-5-(4-fluorophenyl)-1,3,4-thiadiazole | aldrich |
| 2-Amino-5-(4-pyridinyl)-1,3,4-thiadiazole | aldrich |
| N-Ethyl-N-(2-hydroxyethyl)-p-phenylenediamine sulfate salt monohydrate | tci-us |
| 2-(2-Aminophenyl)-1H-benzimidazole | alfa/lancaster |
| 4-(4-Aminophenyl)benzonitrile | tci-us |
| 4-Amino-6-chloro-2-(methylthio)pyrimidine | aldrich |
| 1-Amino-9-fluorenone | aldrich |
| 4-Amino-2-chloro-5-fluoropyrimidine | sigma |
| 2,3-Dichloroaniline | aldrich |
| 2,4,5-TRIMETHYLANILINE | apollo |
| Sulfamethizole | sigma |
| 2-(Aminomethyl)benzimidazole dihydrochloride hydrate | aldrich |
| 9-Aminofluorene hydrochloride | aldrich |
| 4-Aminomethylbenzenesulfonamide hydrochloride | sigma |
| 3,4-Dihydroxybenzylamine hydrobromide | aldrich |
| 9-Aminoacridine hydrochloride hydrate | aldrich |
| 2-Aminobenzophenone | aldrich |
| 4-Aminobenzophenone | aldrich |
| 4-Aminobenzyl cyanide | aldrich |
| 2-Aminobiphenyl | sial |
| 1-Amino-4-bromonaphthalene | aldrich |
| 3-Amino-4-carbethoxypyrazole | aldrich |
| 2-Amino-5-chlorobenzophenone | aldrich |
| 4-Chloro-3-(trifluoromethyl)aniline | aldrich |
| 2-Amino-5-chlorobenzoxazole | aldrich |
| 1-Amino-4-chloronaphthalene | aldrich |
| 2-Amino-5-chloropyridine | aldrich |
| 3-Amino-2-chloropyridine | aldrich |
| 2-Amino-4,6-dichloropyrimidine | aldrich |
| 2-Amino-5,6-dimethylbenzimidazole | aldrich |
| 2-Amino-5,6-dimethylbenzothiazole | aldrich |
| 2-Amino-4,6-dimethylpyridine | aldrich |
| 2-Amino-4,6-dimethylpyrimidine | aldrich |
| 2-Amino-6-ethoxybenzothiazole | aldrich |
| 2-Aminofluorene | aldrich |
| 2-Amino-9-fluorenone | aldrich |
| 5-Aminoindazole | aldrich |
| 6-Aminoindazole | aldrich |
| 5-Aminoindole | aldrich |
| 5-Amino-2-methylbenzothiazole dihydrochloride | aldrich |
| 1-Acetyl-6-aminoindoline | sigma |
| 4-Aminoquinaldine | aldrich |
| 5-Aminoquinoline | aldrich |
| 9-Amino-1,2,3,4-tetrahydroacridine hydrochloride hydrate | aldrich |
| 3,4-Dimethoxyaniline | aldrich |
| 2-Aminobenzonitrile | aldrich |
| DL-Aminoglutethimide | sigma |
| 7-Amino-4-methylcoumarin | sigma |
| Butacaine | sigma |
| 3-Amino-5-tert-butylisoxazole | alfa/lancaster |
| Benoxinate hydrochloride | sigma |
| Bromopride | sigma |
| 5-Chloro-2,4-dimethoxyaniline | tci-us |
| 3,5-Dimethoxyaniline | aldrich |
| Dimethyl 5-aminoisophthalate | aldrich |
| 2,6-Dichloroaniline | aldrich |
| 3,5-Dichloroaniline | aldrich |
| N4-Ethyl-N4-(2-hydroxyethyl)-2-methyl-1,4-phenylenediamine sulfate salt | sigma |
| N-(4-Amino-2,5-diethoxyphenyl)benzamide | sigma |
| 1-(3-Aminophenyl)ethanol | aldrich |
| 2-Morpholinoaniline | alfa/lancaster |
| 4-Amino-N-methyl-alpha-toluenesulfonamide | alfa/lancaster |
| 4-Bromo-3-methoxyaniline | alfa/lancaster |
| Metoclopramide hydrochloride | sigma |
| 4-Methoxy-2-naphthylamine | sigma |
| 4-Phenoxyaniline | aldrich |
| 3-Amino-4-propoxy-benzoic acid 2-diethylamino-ethyl ester hydrochloride | sigma |
| Procainamide hydrochloride | sigma |
| Procaine hydrochloride | sigma |
| Riluzole | sigma |
| Sulfathiazole | tci-us |
| SULFISOMIDINE | tci-us |
| Sulfameter | sigma |
| 4-Amino-n-(6-methoxy-pyrimidin-4-yl)-benzenesulfonamide | sigma |
| Sulfaphenazole | sigma |
| Sulfapyridine | sigma |
| Sulfamethazine | sigma |
| Sulfisoxazole | sigma |
| Sulfadimethoxine | sigma |
| Sulfamethoxazole | sigma |
| Sulfadiazine | sigma |
| Sulfamerazine | sigma |
| Sulfachloropyridazine | sigma |
| 4-Trifluoroacetyl-p-phenylenediamine | sigma |
| 3,4,5-Trimethoxyaniline | aldrich |
| (S)-(−)-1-(2-Naphthyl)ethylamine | fluka |
| (R)-(+)-1-(2-Naphthyl)ethylamine | fluka |
| 3-Chloro-4-methoxybenzylamine hydrochloride | aldrich |
| 5-(Aminomethyl)indole | aldrich |
| 4-(Aminomethyl)benzonitrile hydrochloride | aldrich |
| 3-Hydroxy-4-methoxybenzylamine hydrochloride | aldrich |
| (S)-(−)-2-Amino-1,1,2-triphenylethanol | aldrich |
| (R)-(+)-1,2,2-Triphenylethylamine | aldrich |
| (S)-(−)-1,2,2-Triphenylethylamine | aldrich |
| 4-Phenylbenzylamine | aldrich |
| (R)-(−)-2-Phenylglycinol | aldrich |
| 4-Nitrobenzylamine hydrochloride | aldrich |
| 3-Nitrobenzylamine hydrochloride | aldrich |
| 2-Bromobenzylamine hydrochloride | aldrich |
| 2-Nitrobenzylamine hydrochloride | aldrich |
| 3,5-Bis(trifluoromethyl)benzylamine | aldrich |
| (S)-(+)-2-Phenylglycinol | aldrich |
| p-Dimethylaminobenzylamine dihydrochloride | aldrich |
| 2,4,6-Trimethoxybenzylamine hydrochloride | aldrich |
| (R)-(−)-2-Phenylglycine methyl ester hydrochloride | aldrich |
| 3-Bromobenzylamine hydrochloride | aldrich |
| (1S,2R)-(+)-2-Amino-1,2-diphenylethanol | aldrich |
| (1R,2S)-(−)-2-Amino-1,2-diphenylethanol | aldrich |
| (R)-alpha-Methyl-4-nitrobenzylamine hydrochloride | aldrich |
| (S)-alpha-Methyl-4-nitrobenzylamine hydrochloride | aldrich |
| 2[2-(Aminomethyl)phenylthio]benzyl alcohol | aldrich |
| (1S,2R)-(−)-cis-1-Amino-2-indanol | aldrich |
| (1R,2S)-(+)-cis-1-Amino-2-indanol | aldrich |
| Diethyl (alpha-aminobenzyl)phosphonate hydrochloride | aldrich |
| Methyl 4-(aminomethyl)benzoate hydrochloride | aldrich |
| (1S,2S)-(+)-N-p-Tosyl-1,2-diphenylethylenediamine | aldrich |
| 5-Bromo-2-fluorobenzylamine hydrochloride | aldrich |
| L-p-Hydroxyphenylglycine methyl ester hydrochloride | aldrich |
| (R)-Amino-(4-hydroxyphenyl)acetic acid methyl ester hydrochloride | aldrich |
| 1-(N-Boc-aminomethyl)-4-(aminomethyl)benzene | aldrich |
| (S)-(+)-2-Phenylglycine methyl ester hydrochloride | aldrich |
| (1R,2R)-(−)-N-p-Tosyl-1,2-diphenylethylenediamine | aldrich |
| 2-(Phenylthio)aniline | fluka |
| Sulfamoxole | sigma |
| N-(4-Aminophenyl)piperidine | aldrich |
| 2-Chloro-5-(trifluoromethyl)aniline | fluka |
| 2,5-Dimethylaniline | aldrich |
| tert-Butyl 2-aminobenzoate | fluka |
| 4-Butylaniline | aldrich |
| 2-Bromo-4-methylaniline | aldrich |
| 2-Chloro-4-methylaniline | aldrich |
| 2,4,6-Trimethylaniline | aldrich |
| 3,5-Dimethylaniline | aldrich |
| 3-Aminothiophenol | aldrich |
| 3-(Methylthio)aniline | aldrich |
| 2-Ethyl-6-methylaniline | aldrich |
| 3,4-(Methylenedioxy)aniline | aldrich |
| 4-Isopropylaniline | aldrich |
| 3-Ethylaniline | aldrich |
| 3-Bromoaniline | aldrich |
| 4-Fluoro-2-methylaniline | aldrich |
| 2-Aminophenethyl alcohol | aldrich |
| 1,4-Benzodioxan-6-amine | aldrich |
| 2-Isopropenylaniline | aldrich |
| m-Phenetidine | aldrich |
| 4-tert-Butylaniline | aldrich |
| 4-Chloro-2-(trifluoromethyl)aniline | aldrich |
| 4-Fluoro-2-(trifluoromethyl)aniline | aldrich |
| 4-Fluoro-3-(trifluoromethyl)aniline | aldrich |
| 2-Bromo-5-(trifluoromethyl)aniline | aldrich |
| 3-Fluoro-2-methylaniline | aldrich |
| 4-Butoxyaniline | aldrich |
| 2-Propylaniline | aldrich |
| 4-Propylaniline | aldrich |
| 2,3-Difluoroaniline | aldrich |
| 2-Isopropylaniline | aldrich |
| 4-Bromo-2-(trifluoromethyl)aniline | aldrich |
| 4-Chloro-2-fluoroaniline | aldrich |
| 2-Chloro-4-fluoroaniline | aldrich |
| 2,5-Bis(trifluoromethyl)aniline | aldrich |
| 2-Fluoro-4-methylaniline | aldrich |
| 3-(Trifluoromethoxy)aniline | aldrich |
| 2-Fluoro-6-(trifluoromethyl)aniline | aldrich |
| 2-Fluoro-3-(trifluoromethyl)aniline | aldrich |
| 3-Bromo-4-methylaniline | aldrich |
| 2-tert-Butylaniline | aldrich |
| 2-sec-Butylaniline | aldrich |
| 2-Bromo-4-(trifluoromethoxy)aniline | aldrich |
| 2-(Trifluoromethoxy)aniline | aldrich |
| 4-(Trifluoromethylthio)aniline | aldrich |
| 2-(Difluoromethoxy)aniline | aldrich |
| 3-Bromo-2-methylaniline | aldrich |
| Methyl 3-amino-2-methylbenzoate | aldrich |
| 3-Amino-5-bromobenzotrifluoride | aldrich |
| 3-(Difluoromethoxy)aniline | aldrich |
| 4-Chloro-2-methylaniline | aldrich |
| 2-Bromo-3-methylaniline | aldrich |
| o-Phenetidine | aldrich |
| 2-(Trifluoromethyl)aniline | aldrich |
| 3-(Trifluoromethyl)aniline | aldrich |
| 5-Amino-2-methoxypyridine | aldrich |
| 2-Amino-4-methylthiazole | aldrich |
| 2-Bromoaniline | aldrich |
| 2-Chloro-6-methylaniline | aldrich |
| 5-Chloro-2-methylaniline | aldrich |
| 2,4-Difluoroaniline | aldrich |
| 2,4-Dimethoxyaniline | aldrich |
| Ethyl 3-aminobenzoate | aldrich |
| 2-Ethylaniline | aldrich |
| 4-Ethylaniline | aldrich |
| 5-Fluoro-2-methylaniline | aldrich |
| 2-Fluoroaniline | aldrich |
| 3-Fluoroaniline | aldrich |
| 4-(Methylthio)aniline | aldrich |
| p-Phenetidine | aldrich |
| 3-Iodobenzylamine | aldrich |
| 3-Methylbenzylamine | aldrich |
| 3-Fluorobenzylamine | aldrich |
| 1-Naphthylmethylamine | aldrich |
| 2-Methylbenzylamine | aldrich |
| 1,2-Diphenylethylamine | aldrich |
| 4-Fluoro-alpha-methylbenzylamine | aldrich |
| 2-Methoxybenzylamine | aldrich |
| 3-Methoxybenzylamine | aldrich |
| 2-Fluorobenzylamine | aldrich |
| 4-Fluorobenzylamine | aldrich |
| (R)-(+)-1-(4-Bromophenyl)ethylamine | fluka |
| (S)-(−)-1-(4-Bromophenyl)ethylamine | fluka |
| 2,3-Dimethoxybenzylamine | aldrich |
| 2-Ethoxybenzylamine | aldrich |
| (R)-(+)-1-(1-Naphthyl)ethylamine | aldrich |
| (S)-(−)-1-(1-Naphthyl)ethylamine | aldrich |
| 1,2,3,4-Tetrahydro-1-naphthylamine | aldrich |
| 2-(Trifluoromethyl)benzylamine | aldrich |
| 4-(Trifluoromethyl)benzylamine | aldrich |
| (±)-1-(1-Naphthyl)ethylamine | aldrich |
| 3,4,5-Trimethoxybenzylamine | aldrich |
| 3,5-Dimethoxybenzylamine | aldrich |
| 4-(Trifluoromethoxy)benzylamine | aldrich |
| 3-Fluoro-5-(trifluoromethyl)benzylamine | aldrich |
| (R)-(+)-alpha,4-Dimethylbenzylamine | aldrich |
| (S)-(−)-alpha,4-Dimethylbenzylamine | aldrich |
| 5-Methylfurfurylamine | aldrich |
| 2,4-Dimethoxybenzylamine | aldrich |
| (R)-(−)-1-Aminoindan | aldrich |
| (S)-(+)-1-Aminoindan | aldrich |
| 4-Bromobenzylamine | aldrich |
| 2,3-Dichlorobenzylamine | aldrich |
| 4-tert-Butylbenzylamine | aldrich |
| Benzhydrylamine | aldrich |
| 1-Aminoindan | aldrich |
| 2-Picolylamine | aldrich |
| 3-Picolylamine | aldrich |
| 4-Picolylamine | aldrich |
| 2-Chlorobenzylamine | aldrich |
| 2,4-Dichlorobenzylamine | aldrich |
| 3,4-Dichlorobenzylamine | aldrich |
| Furfurylamine | aldrich |
| (R)-(−)-1-Aminotetraline | alfa/lancaster |
| 4-Methoxybenzylamine | aldrich |
| 4-Fluoro-3-(trifluoromethyl)benzylamine | salor |
| 4-Fluoro-2-(trifluoromethyl)benzylamine | salor |
| 3,5-Dichlorobenzylamine | salor |
| 4-Isopropylbenzylamine | salor |
| 4-Methyl-alpha-phenylphenethylamine | salor |
| 3,5-Difluorobenzylamine | aldrich |

The above list of starting materials was used to prepare the following chemical structures:

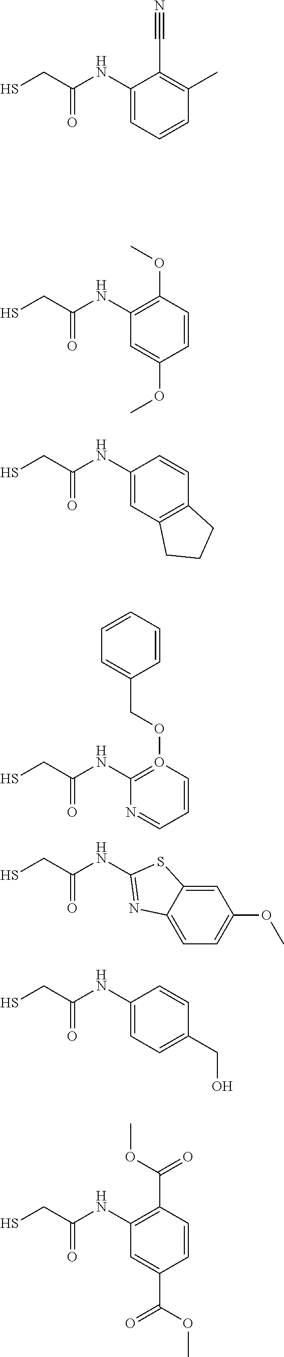

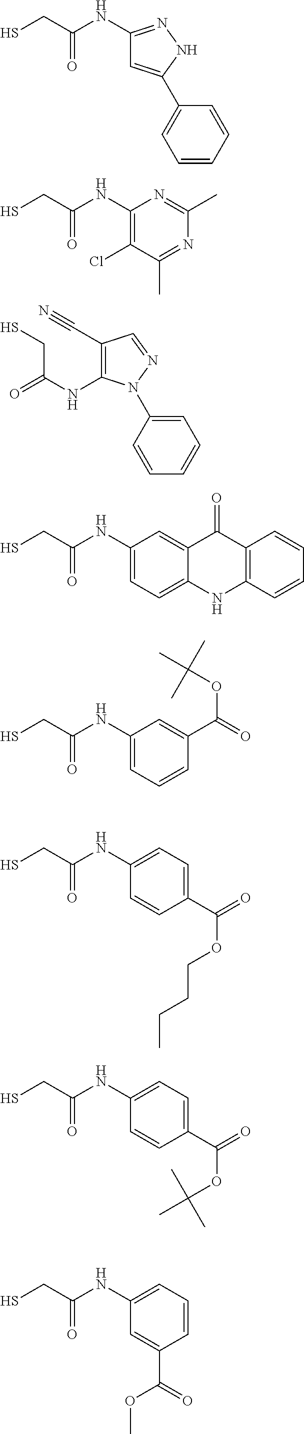

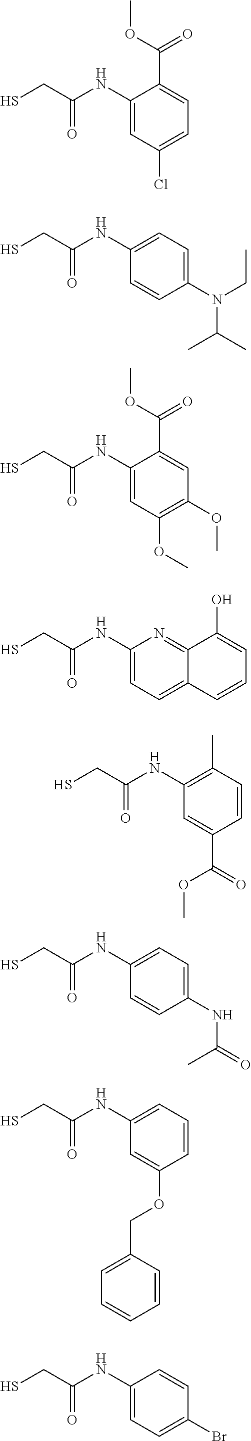

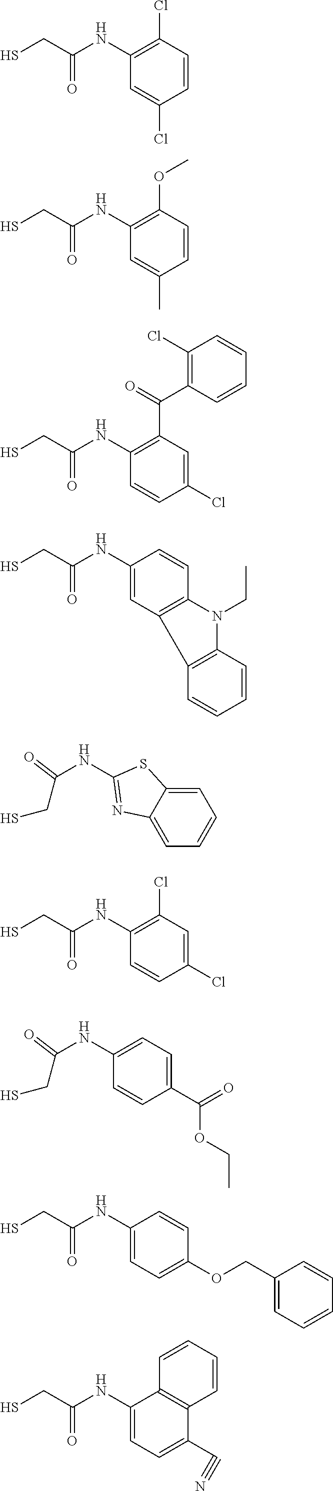

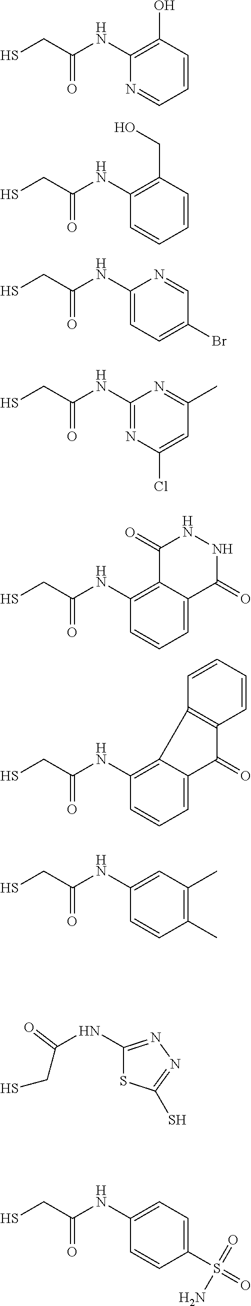

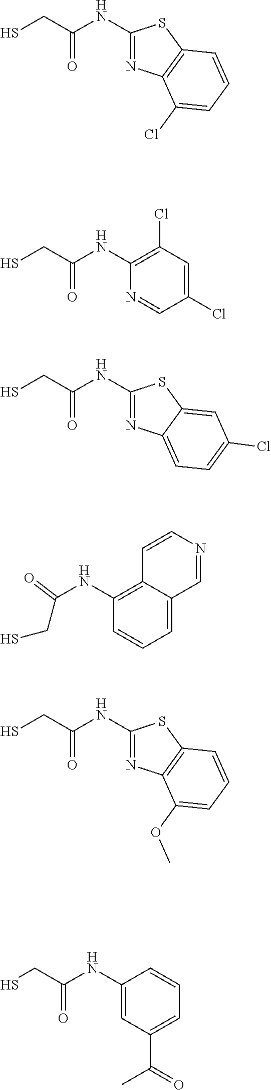

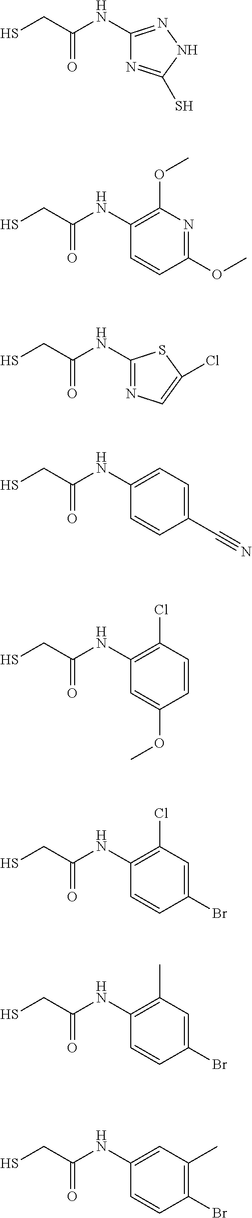

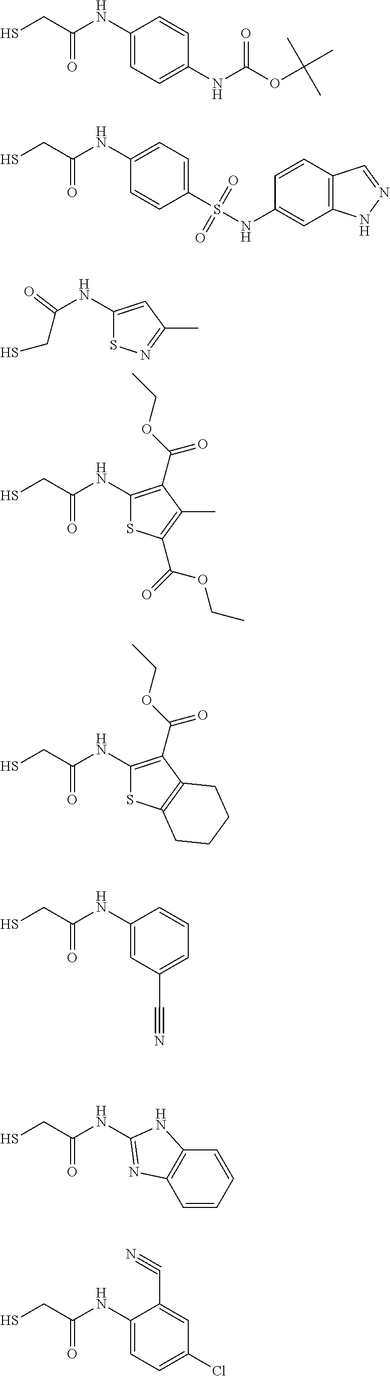

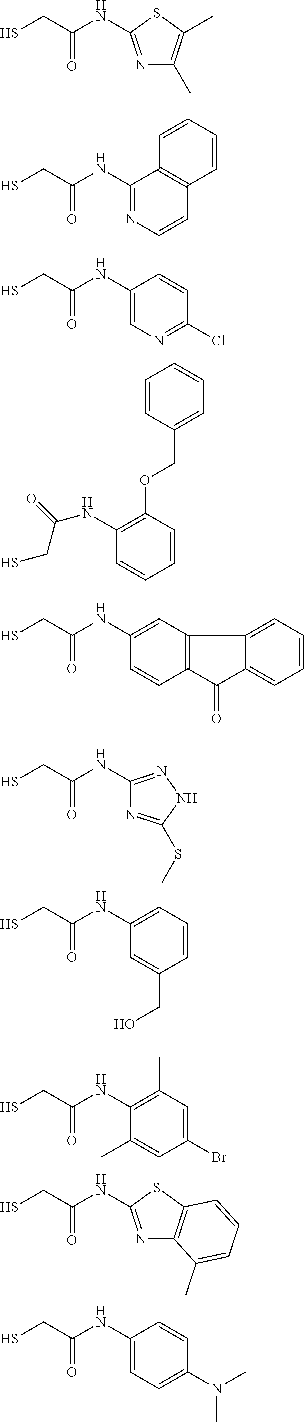

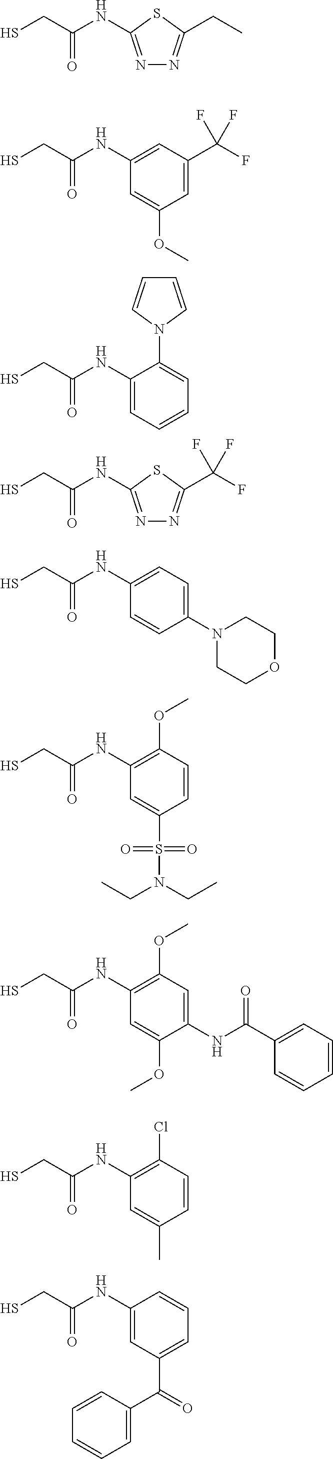

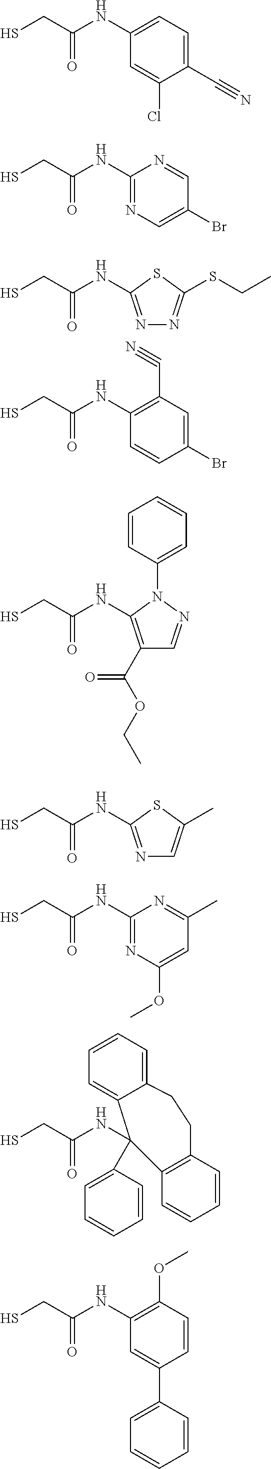

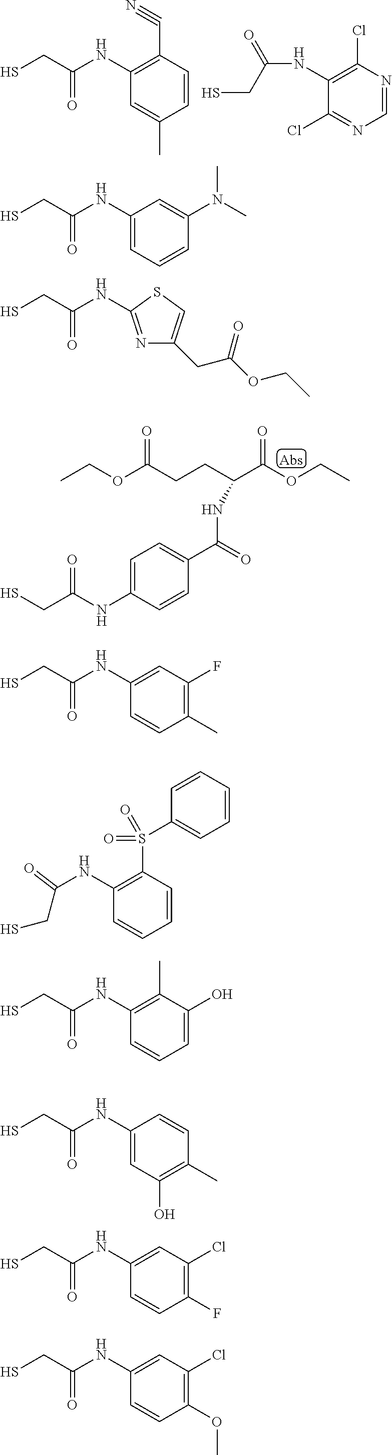

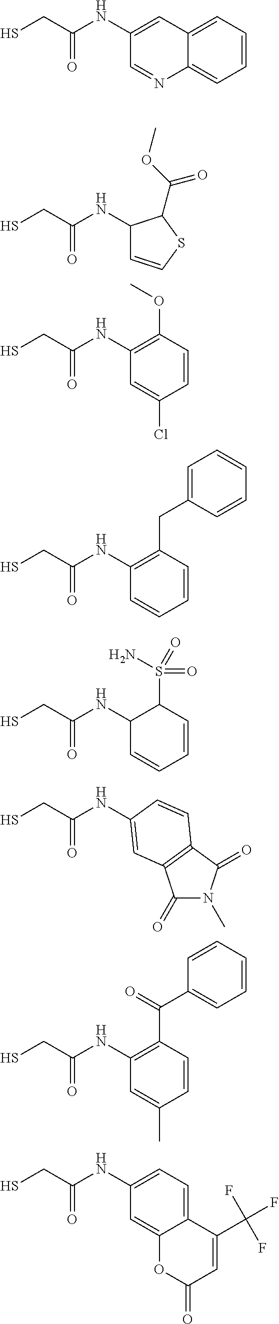

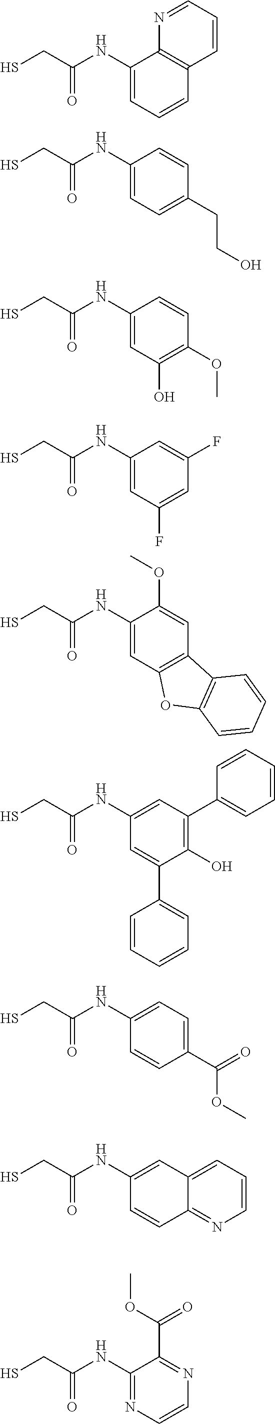

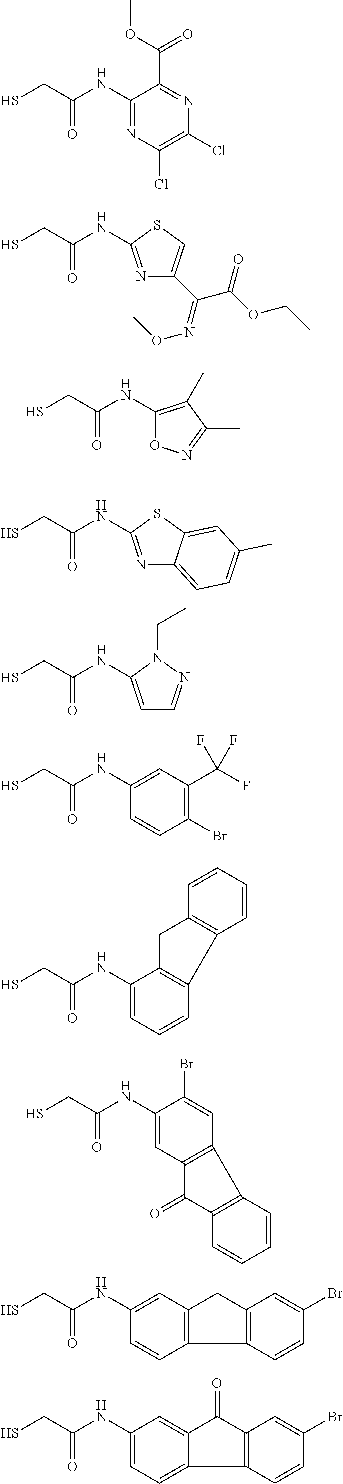

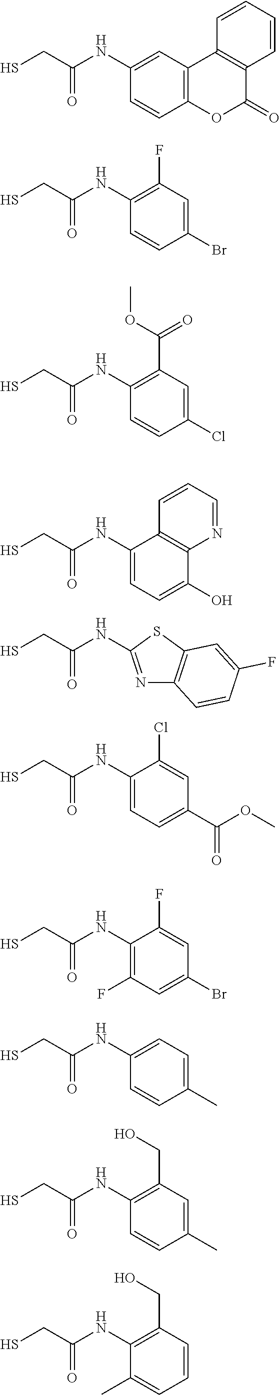

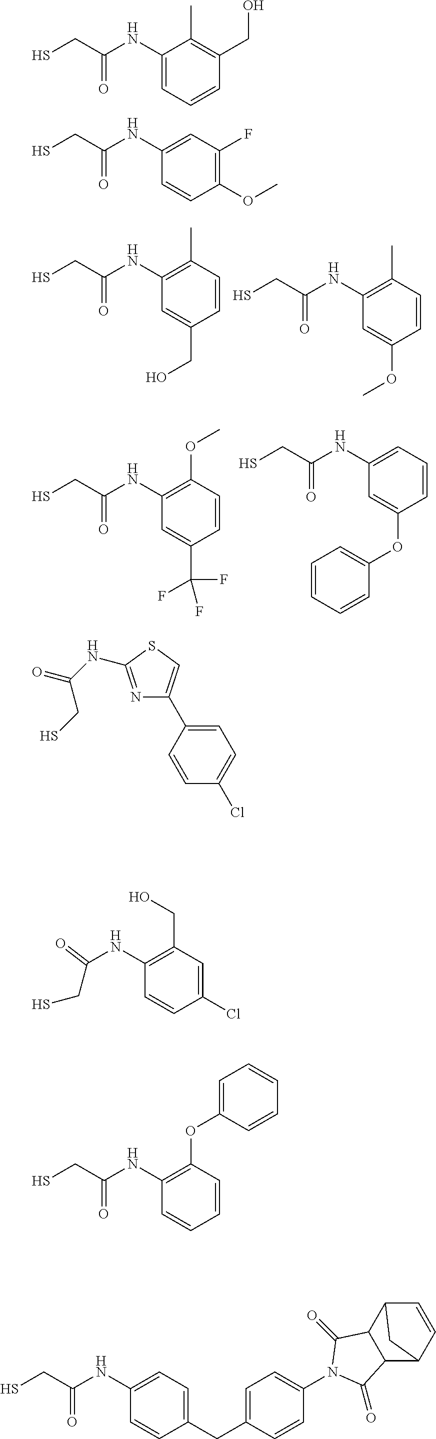

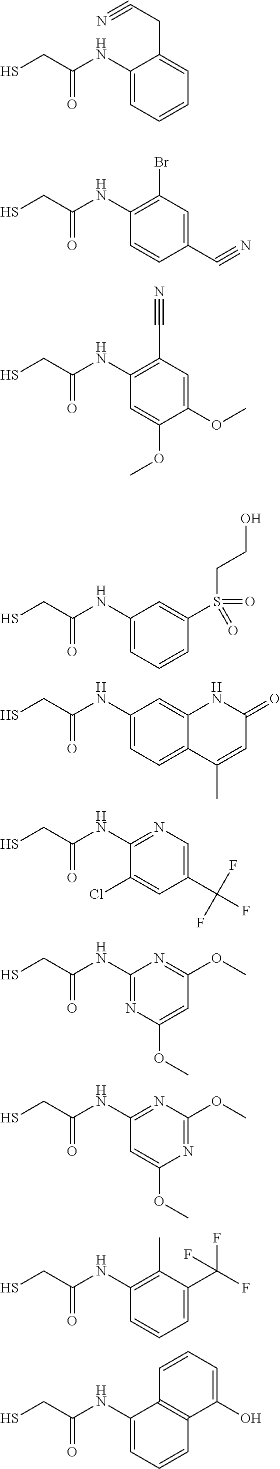

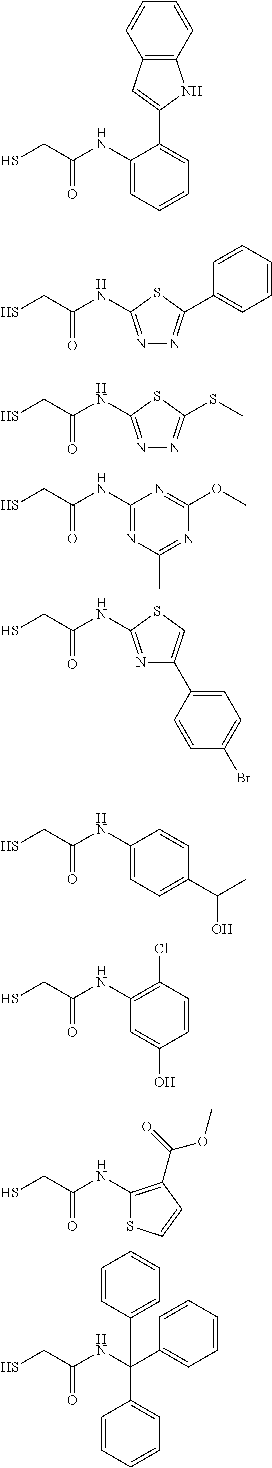

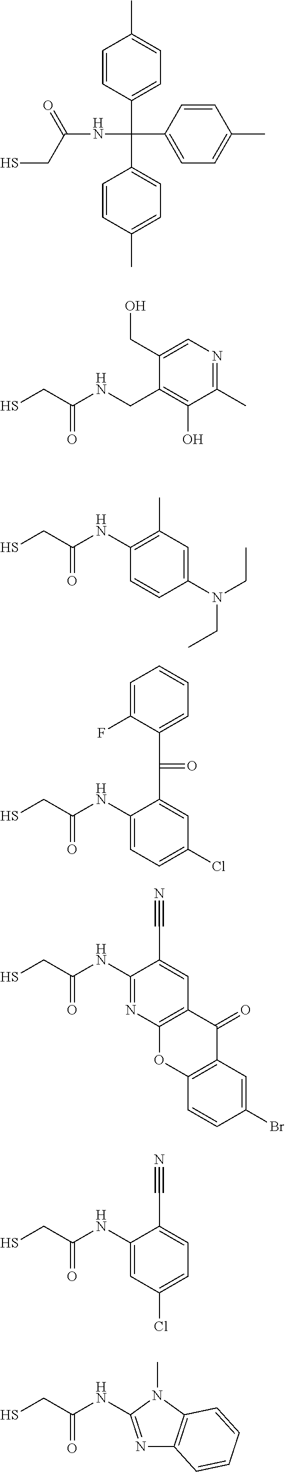

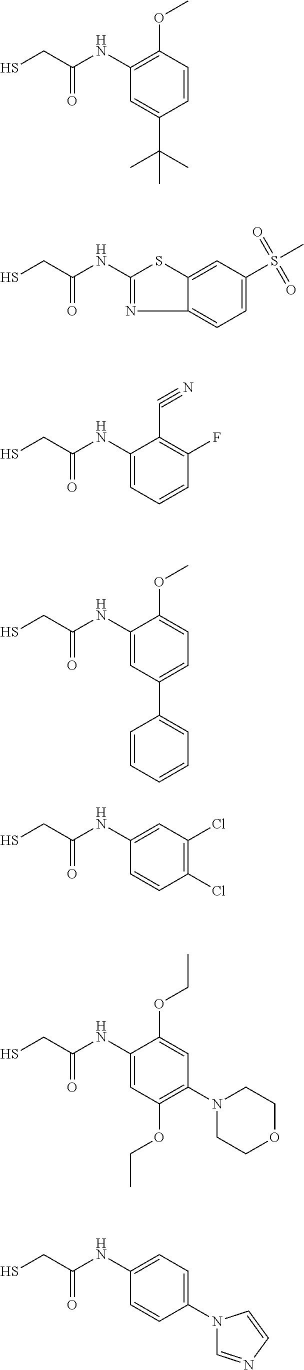

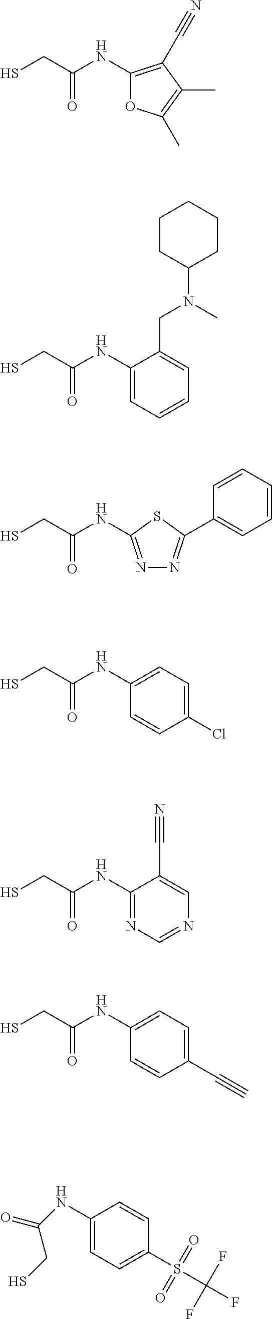

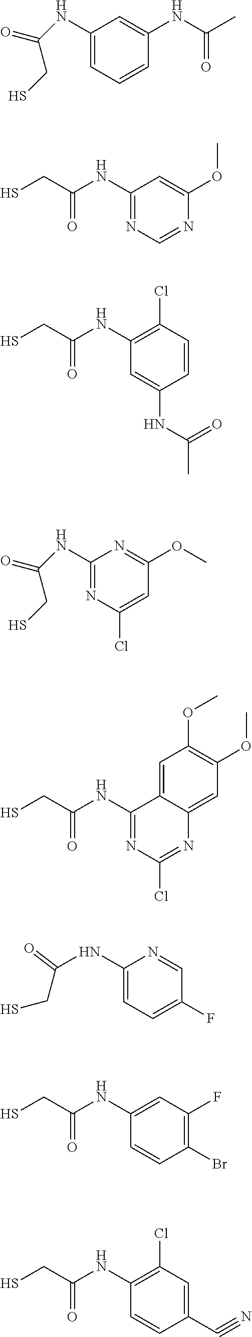

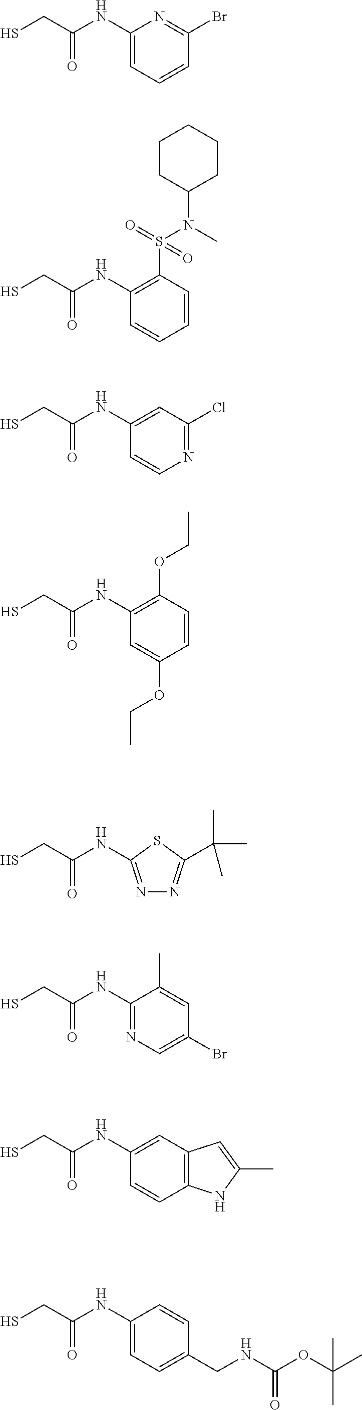

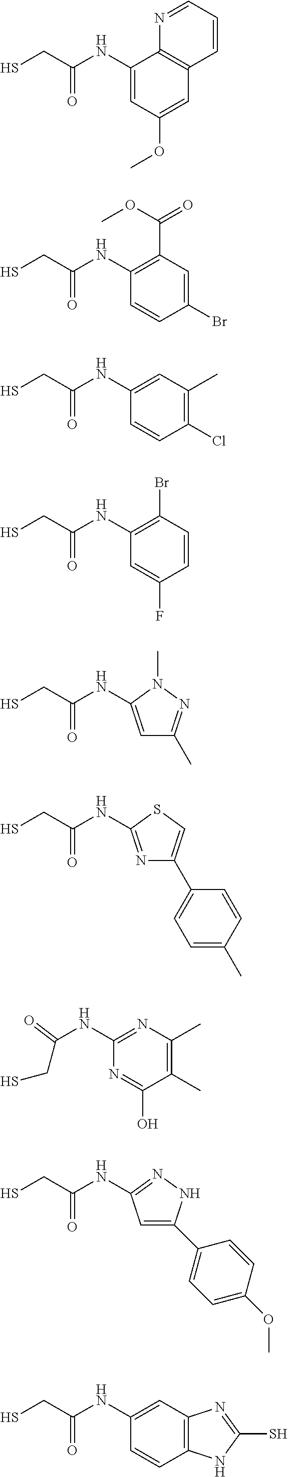

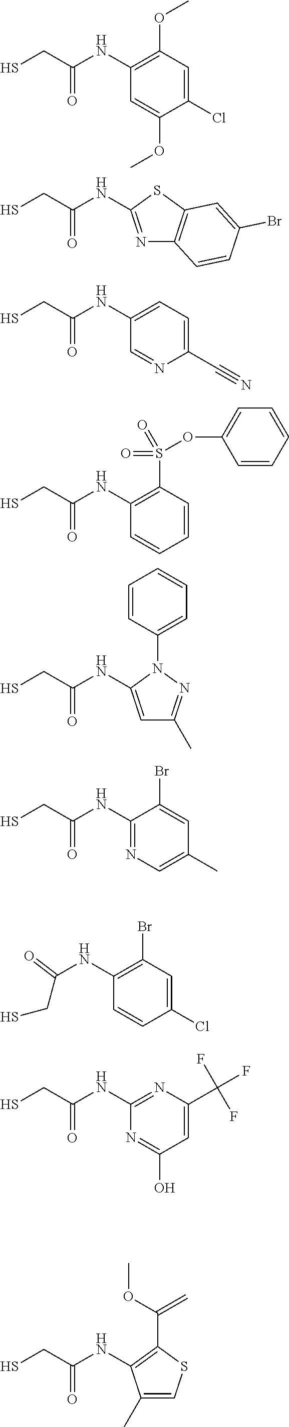

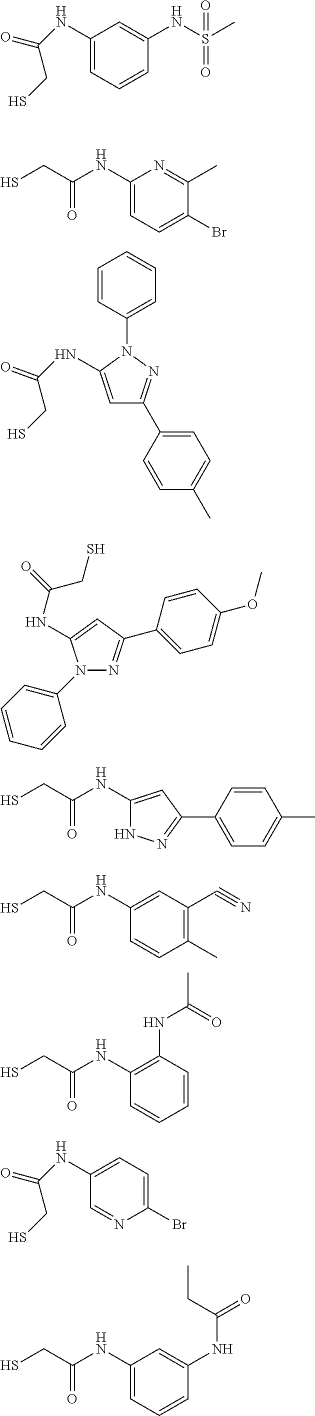

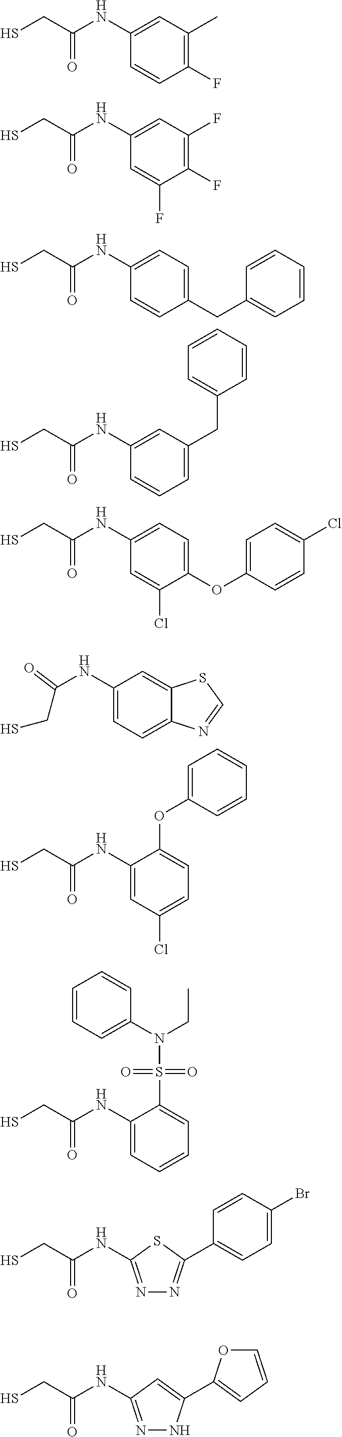

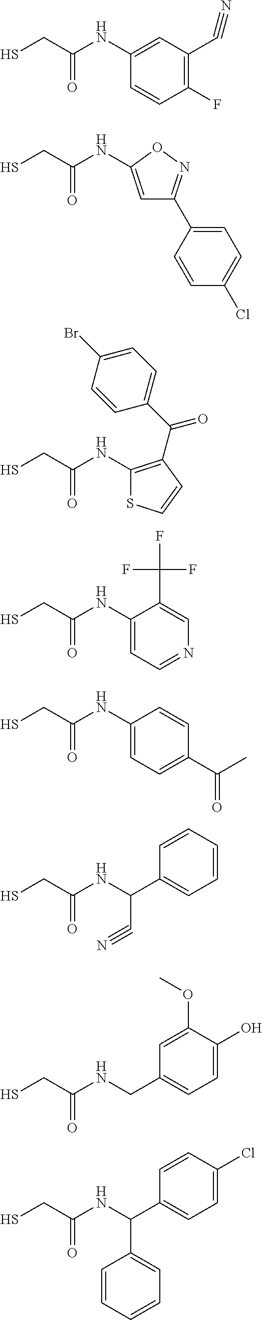

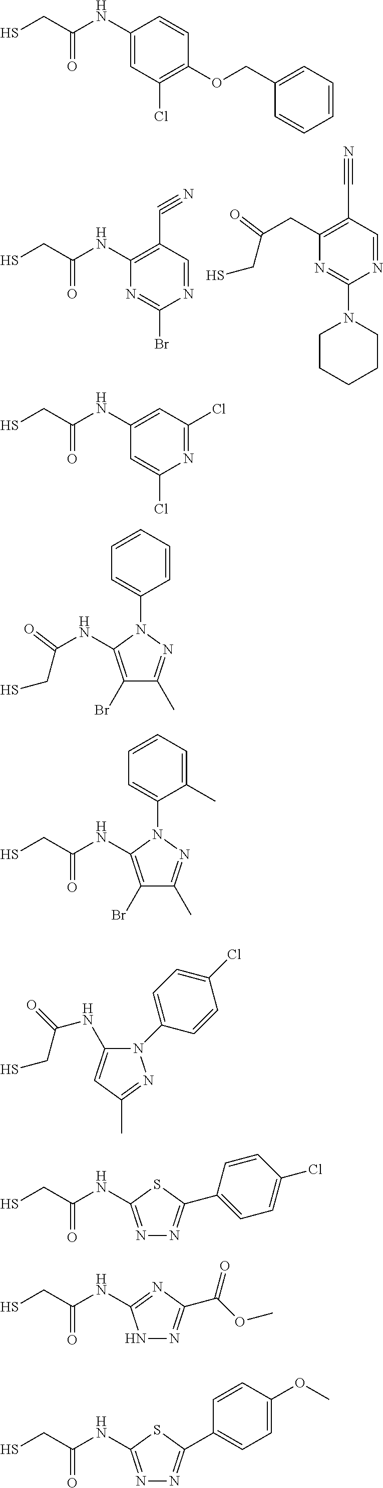

### Example 2

### Mercaptoacetamide Library; Amide Synthesis Procedure

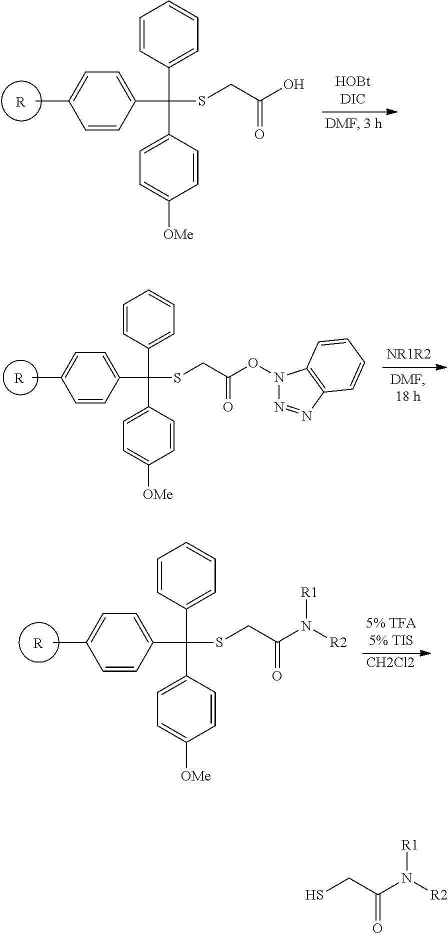

To each of eighty reaction wells of a 96-well FlexChem® Synthesis Reaction Block (Scigene, Corp., Sunnyvale, Calif.) was added 2-carboxyethanethiol 4-methoxytrityl resin (44 mg, 0.0792 mmol, loading 1.8 mmol/g; obtained from Novabiochem, San Diego, Calif.). The resin was swollen by adding CH₂Cl₂ (1 mL/well) to each well and shaking the reaction block for 30 min. The 96-well reaction block was then drained and the resin was washed first with CH₂Cl₂ (1 mL×80 wells) and then with DMF (1 mL×80 wells).

To each well was added a solution of diisopropylcarbodiimide (DIC; 24.5 μL, 0.158 mmol) and hydroxybenzotriazole-hydrate (24.3 mg, 0.158 mmol) in dimethylformamide (DMF, 0.5 mL). The reaction block was shaken for 3 h at room temperature to pre-form the HOBt-activated ester. Stock solutions of eighty different amines (0.15 mmol) in DMF (0.5 mL) were added to each well and the reactor was shaken at room temperature for 18 h. The reaction block was then drained using a vacuum manifold and each well containing resin was washed with DMF (2×1 mL), MeOH (2×1 mL), H2O (2×1 mL), MeOH (2×1 mL), and CH₂Cl₂ (3×1 mL).

The thiol-bound mercaptoacetamide products were then cleaved from the resin. First the resin in each well was swollen by shaking with CH₂Cl₂ (1 mL×80 wells) for 15 min. The solvent was then removed using a vacuum manifold. A cleavage cocktail of 5% trifluoroacetic acid (TFA) and 5% triethylsilane (TIS) in CH₂Cl₂ (1 mL×80 wells) was added to each well, and the reactor was then shaken for 15 min. Next the reactor was placed over the top of a clean deep-well (2 mL) 96-well plate in a collection manifold, vacuum was applied and the product (in solution) was collected. The solvent was allowed to evaporate in a ventilation hood for 8 h, and then placed in a vacuum desiccator (20-100 mm Hg) overnight to remove the trace amounts of TFA and TIS.

### Example 3

### Synthesis of N-(1-(2,4-dichlorophenyl)propyl)-2-mercaptoacetamide

Benzylamine substituted analogs were synthesized using a two-step reaction sequence starting from commercially available phenylketones. The phenylketones were reductively aminated using ammonia and sodium borohydride in the presence of a dehydrating agent to provide benzylamine analogs. The mercaptoacetamides were obtained by refluxing the benzylamine analogs with thioglycolic acid in toluene.

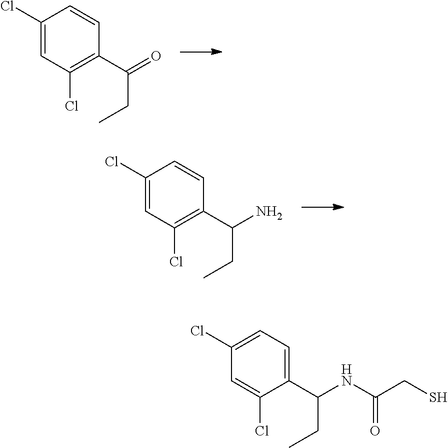

### 1-(2,4-dichlorophenyl)propan-1-amine

To a solution of 2,4-dichloropropiophenone (1.58 mL, 10 mmol) and titanium IV isopropoxide (6 mL, 20 mmol) was added an ice-cold solution of ammonia in methanol (7N, 7 mL, 49 mmol). Sodium borohydride (600 mg, 15 mmol) was added and the reaction was stirred for 3 days. The reaction mixture was poured into 25 mL of 2% NH₄OH, prepared from concentrated NH₄OH (1.7 mL) diluted with H₂O (23 mL). The resulting white solid was removed by filtration, washed with diethyl ether (2×50 mL), and the aqueous layer was extracted with ether. The combined organic layers were extracted with 1N HCl (2×25 mL). The acidic layer was made basic with concentrated NH₄OH and the product was extracted with dichloromethane and dried to provide 900 mg, 44% of a white solid.

### N-(1-(2,4-dichlorophenyl)propyl)-2-mercaptoacetamide

A solution of 1-(2,4-dichlorophenyl)propan-1-amine (900 mg, 4.43 mmol), thioglycolic acid (308 μL, 4.43 mmol), and toluene (30 mL) were heated to reflux under argon for 18 h and the resulting water was removed using a Dean-Stark apparatus. The reaction solution was then cooled to room temperature and the volatiles were removed by rotary-evaporation. The resulting oil was chromatographed on flash column silica gel using a gradient of hexane to 50% ethyl acetate/hexane to provide after evaporation 670 mg, 54.4% yield of product as a white powder.

### Example 4

### Synthesis of N-(2,4-dichlorobenzyl)-2-mercaptopropanamide

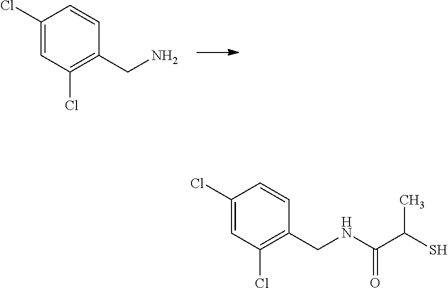

### N-(2,4-dichlorobenzyl)-2-mercaptopropanamide

A solution of 2,4-dichlorobenzylamine (1200 mg, 6.82 mmol), thiolactic acid (604 μL, 6.82 mmol), and toluene (40 mL) were heated to reflux under argon for 18 h and the resulting water was removed using a Dean-Stark apparatus. The reaction solution was then cooled to room temperature and the volatiles were removed by rotary-evaporation. The resulting oil was chromatographed on flash column silica gel using a gradient of hexane to 50% ethyl acetate/hexane to provide after evaporation 1.12 g, 62.24% yield of product as a white powder.

### Example 5

### General Procedure for Coupling Amines with Thioglycolic Acid

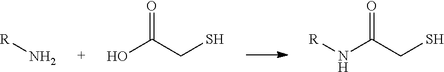

The amine (2 mmol) was weighed into a 10 mL vial, and toluene (2 mL) and thioglycolic acid (4 mmol, 277 uL) were added to the vial. The vial was purged with Argon, capped, and placed in an aluminum reaction block heated to 100° C. for 24 h. After cooling the reaction mixture to room temperature, work up varied based upon the presence or lack of filterable solid. In the situation in which the product precipitated out as a solid, the solid was transferred to a 2 mL glass fritted filter using toluene, rinsed with toluene, H₂O, saturated aqueous NaHCO₃ solution, H₂O, 1N HCl, a minimum amount of acetonitrile, and a minimum amount of diethyl ether. In the situation in which the product remained in solution, the solution was diluted with 6 mL diethyl ether (6 mL), washed 3× with saturated aqueous NaHCO₃, H₂O, 3× with 1N HCl, and brine. The organic solution was dried over anhydrous Na₂SO₄ and evaporated in vacuo. The resulting solid was tritrated with a minimum amount of diethyl ether to yield a solid.

### Example 6

### N-(2-chloro-5-hydroxyphenyl)-2-mercaptoacetamide

N-(2-chloro-5-hydroxyphenyl)-2-mercaptoacetamide was synthesized using the above general procedure by the reaction of a substituted aniline with mercaptoacetic acid at elevated temperature.

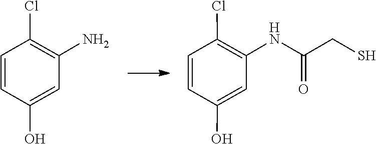

### N-(2-chloro-5-hydroxyphenyl)-2-mercaptoacetamide

Under argon a solution of 3-amino-4-chlorophenol (287 mg, 2 mmol), thioglycolic acid (695 uL, 10 mmol), and toluene (2 mL) were heated to 100° C. in a sealed tube using an aluminum reaction block heater-stirrer. After 24 h the reaction was cooled to room temperature, and the resulting precipitate was collected on fritted glass and washed with toluene. The solid was dried under high vacuum (1 mm) at room temperature to provide 335 mg, 46% yield of a light-gray powder.

### Example 7

### Synthesis of N-(3-(4-chlorophenyl)-1H-pyrazol-5-yl)-2-mercaptoacetamide

N-(3-(4-chlorophenyl)-1H-pyrazol-5-yl)-2-mercaptoacetamide was synthesized using the above general procedure by the reaction of an aminopyrazole with mercaptoacetic acid at elevated temperature.

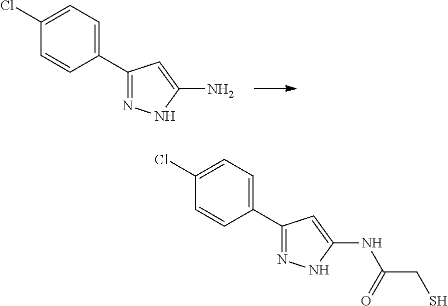

### N-(3-(4-chlorophenyl)-1H-pyrazol-5-yl)-2-mercaptoacetamide

Under argon a solution of 5-amino-3-(4-chlorophenyl)pyrazole (1.93 g, 10 mmol), thioglycolic acid (1.4 mL, 20 mmol), and toluene (10 mL) were heated to 100° C. in a sealed tube using an aluminum reaction block heater-stirrer. After 24 h the reaction was cooled to room temperature, and the resulting precipitate was collected on fritted glass and washed with toluene, saturated aqueous NaHCO₃, H₂O, acetonitrile, and diethyl ether. The solid was dried under high vacuum (1 mm) at room temperature to provide 2.30 g, 86% yield of a white powder.

### Example 8

### Synthesis of 2-Mercapto-N-(3-(thiophen-2-yl)-1H-pyrazol-5-yl)acetamide

2-Mercapto-N-(3-(thiophen-2-yl)-1H-pyrazol-5-yl)acetamide was synthesized using the above general procedure by the reaction of an aminopyrazole with mercaptoacetic acid at elevated temperature.

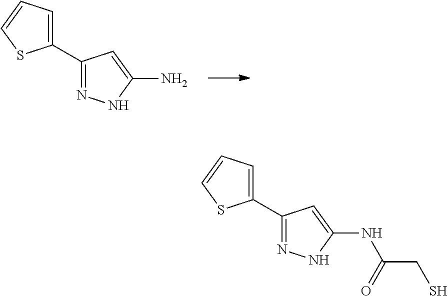

### 2-Mercapto-N-(3-(thiophen-2-yl)-1H-pyrazol-5-yl)acetamide

Under argon a solution of 5-amino-3-(2-thienyl)pyrazole (330 mg, 2 mmol), thioglycolic acid (554 uL, 8 mmol), and toluene (2 mL) were heated to 100° C. in a sealed tube using an aluminum reaction block heater-stirrer. After 30 h the reaction was cooled to room temperature, and the resulting precipitate was collected on fritted glass and washed with toluene, saturated aqueous NaHCO₃, acetonitrile, and diethyl ether. The solid was dried under high vacuum (1 mm) at room temperature to provide 248 mg, 52% yield of a light-gray powder.

### Example 9

### Synthesis of 2-Mercapto-N-((R)-1-(naphthalen-7-yl)ethyl)acetamide

2-Mercapto-N-((R)-1-(naphthalen-7-ypethyl)acetamide was synthesized using the above general procedure by the reaction of a primary amine with mercaptoacetic acid at elevated temperature.

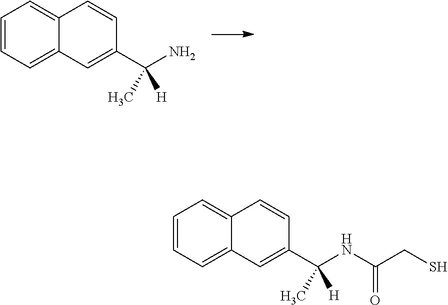

### 2-Mercapto-N-((R)-1-(naphthalen-7-yl)ethyl)acetamide

Under argon a solution of 5-amino-3-(4-chlorophenyl)pyrazole (1.93 g, 10 mmol), thioglycolic acid (1.4 mL, 20 mmol), and toluene (10 mL) were heated to 100° C. in a sealed tube using an aluminum reaction block heater-stirrer. After 24 h the reaction was cooled to room temperature, and the resulting precipitate was collected on fitted glass and washed with toluene, saturated aqueous NaHCO₃, H₂O, acetonitrile, and diethyl ether. The solid was dried under high vacuum (1 mm) at room temperature to provide 2.30 g, 86% yield of a white powder.

### Example 10

### Synthesis of N-(3-(4-chlorophenyl)-4-methyl-1H-pyrazol-5-yl)-2-mercaptoacetamide

A four-step reaction sequence starting from commercially available 4-chlorobenzoic acid was used to synthesize 4-Substituted analogs. After esterification, the methyl ester was condensed with an appropriately substituted nitrile such as propionitrile to give, for example, an alpha-methyl-beta-ketonitrile. The 4-methylpyrazole was obtained by cyclization with hydrazine. The mercaptoacetamide analog was obtained by refluxing the 3-aminopyrazole with thioglycolic acid in toluene.

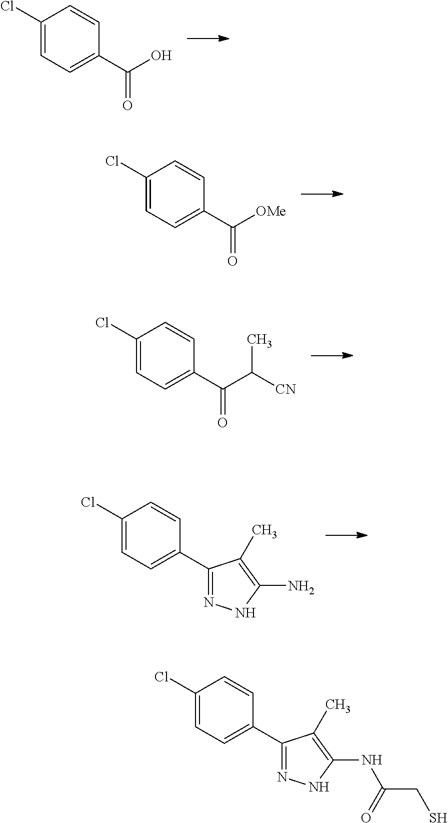

### Methyl 4-chlorobenzoate

To 4-chlorobenzoic acid (15.6 g, 100 mmol) dissolved in methanol (100 mL) was added concentrated H₂SO₄ (1 mL). The reaction was then heated to reflux for 18 h. The reaction mixture was then cooled in an ice bath, and the crystalline product was collected on fritted glass, washed with water, saturated aqueous NaHCO₃, and water. The material was further dried under high vacuum to give 15.3 g, 90% yield of a white crystalline solid.

### 3-(4-Chlorophenyl)-2-methyl-3-oxopropanenitrile

Methyl 4-chlorobenzoate (1.70 g, 10 mmol) was dissolved in propionitrile (10 mL, dried over 3 A molecular sieves), and NaOCH₃ (1.08 g, 20 mmol) was added and the reaction was stirred at room temperature under argon for 18 h. The reaction was heated to 100° C. for 1 h, and the reaction mixture was then cooled to ambient temperature and the volatiles were removed by rotary evaporation leaving a residue. The residue was dissolved in water (10 mL) and washed with ether (3 times). The aqueous layer was then acidified to pH 6.4 with citric acid. The resulting precipitate was collected on fritted glass, washed with water, saturated aqueous NaHCO₃, and water. The solid on the filter was dried under high vacuum over night to provide the beta-ketonitrile (428 mg, 48% yield).

### 3-(4-Chlorophenyl)-4-methyl-1H-pyrazol-5-amine

The beta-ketonitrile, prepared above, 3-(4-chlorophenyl)-2-methyl-3-oxopropanenitrile (353 mg, 2 mmol), was dissolved in abs. ethanol (2 mL). To this solution was added anhydrous hydrazine (75 μL, 2.4 mmol), and the reaction was stirred for 1 h at room temperature allowing the hydrazone to form and precipitate. The mixture was then heated to 100° C. for 45 min., and the progress of the reaction was monitored by HPLC. After heating for 45 min., water (1 mL to 5 mL) was added to precipitate the heterocyclic product as a solid. This material was collected on a fitted glass funnel, washed with water, then dried overnight under high vacuum to provide 267.5 mg, 644% yield of a white powder.

### N-(3-(4-Chlorophenyl)-4-methyl-1H-pyrazol-5-yl)-2-mercaptoacetamide

A solution of 3-(4-chlorophenyl)-4-methyl-1H-pyrazol-5-amine (207.5 mg, 1 mmol), thioglycolic acid (104 μL, 1.5 mmol), and toluene (0.5 mL) were heated in a sealed tube under argon for 24 h. The reaction solution was then cooled to room temperature to precipitate the crude product as a solid. The solid was collected on a fritted glass funnel, and washed with water (2×2 mL), saturated aqueous NaHCO₃ (3×2 mL), water (3×2 mL), 5% HCl (3×2 mL), water (2×2 mL), acetonitrile (1 ml), and diethyl ether (1 mL). The washed product was dried overnight under high vacuum to provide 201.0 mg, 71% yield of a white powder.

### Example 11

### Synthesis of N-(3-(4-chlorophenyl)-4-ethyl-1H-pyrazol-5-yl)-2-mercaptoacetamide

A four-step reaction sequence starting from commercially available 4-chlorobenzoic acid was used to synthesize 4-Substituted analogs. After esterification, the methyl ester was condensed with an appropriately substituted nitrile such as butyronitrile to give, for example, an alpha-ethyl-beta-ketonitrile. The 4-ethylpyrazole was obtained by cyclization with hydrazine. The mercaptoacetamide analog was obtained by refluxing the 3-aminopyrazole with thioglycolic acid in toluene.

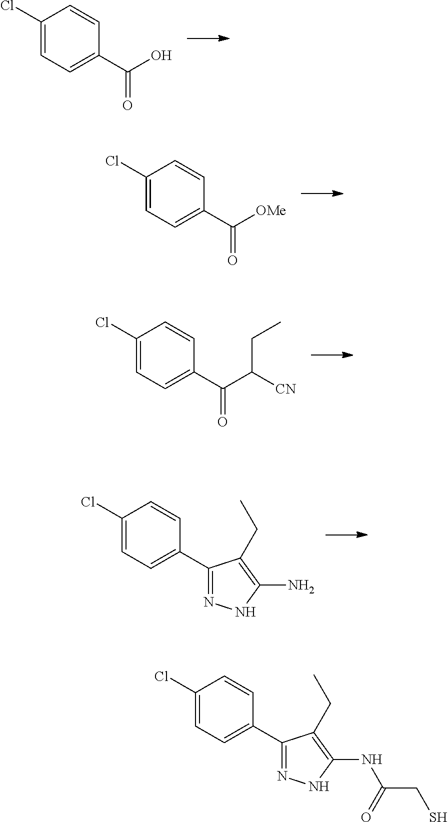

### Methyl 4-chlorobenzoate

To 4-chlorobenzoic acid (15.6 g, 100 mmol) dissolved in methanol (100 mL) was added concentrated H₂SO₄ (1 mL), and the reaction was then heated to reflux for 18 h. The reaction mixture was then cooled in an ice bath, and the crystalline product was collected on fritted glass, washed with water, saturated aqueous NaHCO₃, and water. The material was further dried under high vacuum to give 15.3 g, 90% yield of a white crystalline solid.

### 3-(4-chlorophenyl)-2-ethyl-3-oxopropanenitrile

Methyl 4-chlorobenzoate (1.70 g, 10 mmol) was dissolved in butyronitrile (10 mL, dried over 3 A molecular sieves). NaOCH₃ (1.08 g, 20 mmol) was added and the reaction was stirred at room temperature under argon for 18 h, and the reaction was then heated to 10° C. for 1 h. The reaction mixture was cooled to ambient temperature and the volatiles were removed by rotary evaporation. The residue was dissolved in water (10 mL) and washed with ether (3 times), and the aqueous layer was then acidified to pH 6.4 with citric acid. The resulting precipitate was collected on fitted glass, washed with water, saturated aqueous NaHCO₃, and water. The solid on the filter was dried under high vacuum over night to provide the beta-ketonitrile (841 mg, 41% yield).

### 3-(4-chlorophenyl)-4-ethyl-1H-pyrazol-5-amine

The beta-ketonitrile, prepared above, 3-(4-chlorophenyl)-2-ethyl-3-oxopropanenitrile (415 mg, 2 mmol), was dissolved in abs. ethanol (2 mL). To this solution was added anhydrous hydrazine (75 μL, 2.4 mmol), and the reaction was stirred for 1 h at room temperature allowing the hydrazone to form and precipitate. The mixture was then heated to 100 C for 45 min, and the progress of the reaction was monitored by HPLC. After heating for 45 min, water (1 mL to 5 mL) was added to precipitate the heterocyclic product as a solid. This material was collected on a fritted glass funnel, washed with water, and dried overnight under high vacuum to provide 303.1 mg, 68.4% yield.

### N-(3-(4-chlorophenyl)-4-ethyl-1H-pyrazol-5-yl)-2-mercaptoacetamide

A solution of 3-(4-chlorophenyl)-4-ethyl-1H-pyrazol-5-amine (221.5 mg, 1 mmol), thioglycolic acid (104 μL, 1.5 mmol), and toluene were heated in a sealed tube under argon for 24 h. The reaction solution was then cooled to room temperature to precipitate the crude product as a solid. The solid was collected on a fritted glass funnel, and washed with water (2×2 mL), saturated aqueous NaHCO₃ (3×2 mL), water (3×2 mL), 5% HCl (3×2 mL), water (2×2 mL), acetonitrile (1 ml), and diethyl ether (1 mL). The washed product was dried overnight under high vacuum to provide 221.3 mg, 74.8% yield of a white powder.

### Example 12

### Synthesis of N-(1-benzyl-3-(4-chlorophenyl)-1H-pyrazol-5-yl)-2-mercaptoacetamide

A four-step reaction sequence starting from commercially available 4-chlorobenzoic acid was used to synthesize 4-Substituted analogs. After esterification, the methyl ester was condensed with acetonitrile to give the 3-aminopyrazole. The N1-benzylpyrazole was obtained by cyclization with benzylhydrazine. The mercaptoacetamide analog was obtained by refluxing the N1-substitutedpyrazole with thioglycolic acid in toluene.

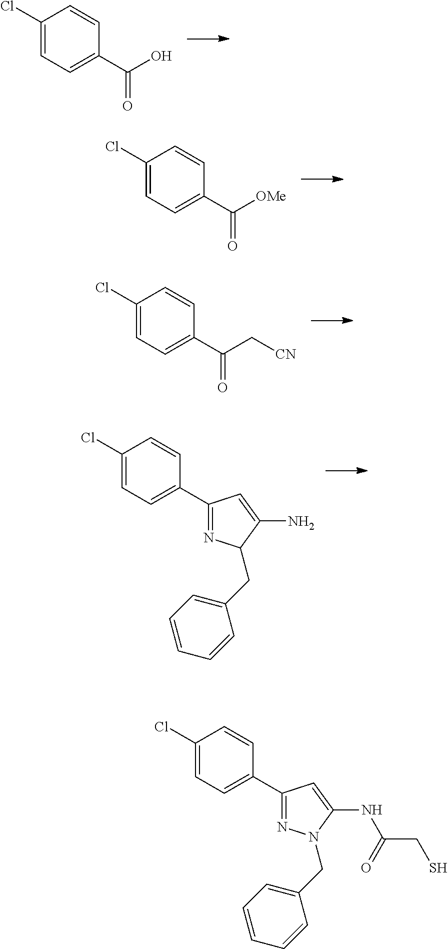

### Methyl 4-chlorobenzoate

To 4-chlorobenzoic acid (15.6 g, 100 mmol) dissolved in methanol (100 mL) was added concentrated H₂SO₄ (1 mL). The reaction was then heated to reflux for 18 h, and the reaction mixture was cooled in an ice bath. The crystalline to product was collected on fritted glass, washed with water, saturated aqueous NaHCO₃, and water. The material was further dried under high vacuum to give 15.3 g, 90% yield of a white crystalline solid.

### 3-(4-Chlorphenyl)-3-oxopropanenitrile

Methyl 4-chlorobenzoate (3.40 g, 20 mmol) was dissolved in toluene (16 mL). Acetonitrile (1.32 mL, 25 mmol) and NaOCH₃ (1.08 g, 20 mmol) were added and the reaction was stirred at room temperature under argon for 18 h. The reaction was heated to 100° C. for 1 h, and the reaction mixture was cooled to ambient temperature and the volatiles were removed by rotary evaporation leaving a residue. The residue was dissolved in water (10 mL) and washed with diether (3 times). The aqueous layer was then acidified to pH 6.4 with citric acid. The resulting precipitate was collected on fitted glass, washed with water, saturated aqueous NaHCO₃, and water. The solid on the filter was dried under high vacuum over night to provide the beta-ketonitrile (692 mg, 20% yield).

### 1-Benzyl-3-(4-chlorophenyl)-1H-pyrazol-5-amine

The beta-ketonitrile, prepared above, 3-(4-chlorophenyl)-3-oxopropanenitrile (177 mg, 1 mmol), was dissolved in abs. ethanol (1 mL). To this solution was added benzyl hydrazine (146, 1.2 mmol, prepared by free basing commercial benzylhydrazine hydrochloride). The reaction mixture was heated in a sealed tube to 100° C. for 1 h, and the reaction was cooled to room temperature. Water (2 mL) was added dropwise to precipitate the heterocyclic product as a solid. This material was collected on a fritted glass funnel, washed with water, then dried overnight under high vacuum to provide 238.8 mg, 85% yield of a fluffy powder.

### N-(1-benzyl-3-(4-chlorophenyl)-1H-pyrazol-5-yl)-2-mercaptoacetamide

A solution of 1-benzyl-3-(4-chlorophenyl)-1H-pyrazol-5-amine (238.0 mg, 0.85 mmol), thioglycolic acid (117 μL, 2.0 mmol), and toluene (850 μL) were heated in a sealed tube under argon for 48 h. The reaction solution was cooled to room temperature to precipitate the crude product as a solid. The solid was collected on a fritted glass funnel, and washed with H₂O (2×2 mL), saturated aqueous NaHCO₃ (3×2 mL), H₂O (3×2 mL), 5% HCl (3×2 mL), water (2×2 mL), acetonitrile (1 ml), and diethyl ether (1 mL). The washed product was dried overnight under high vacuum to provide 173.5 mg, 57% yield of a white powder.

### Example 13

### Synthesis of N-(5-fluoropyridin-2-yl)-2-mercaptoacetamide

An alternative method to synthesize mercaptoacetamide analogs involved coupling of amines and anilines with the para-nitrophenylester (PNP) of S-trityl-mercaptoacetic acid. The PNP-ester was synthesized in three steps from triphenylthiomethanol. The mercaptan was reacted with ethyl bromoacetate, the ethyl ester was cleaved under basic conditions to provide the free carboxylic acid. The acid was coupled with para-nitrophenol to give the PNP-activated, trityl-protected, mercaptoacetic acid.

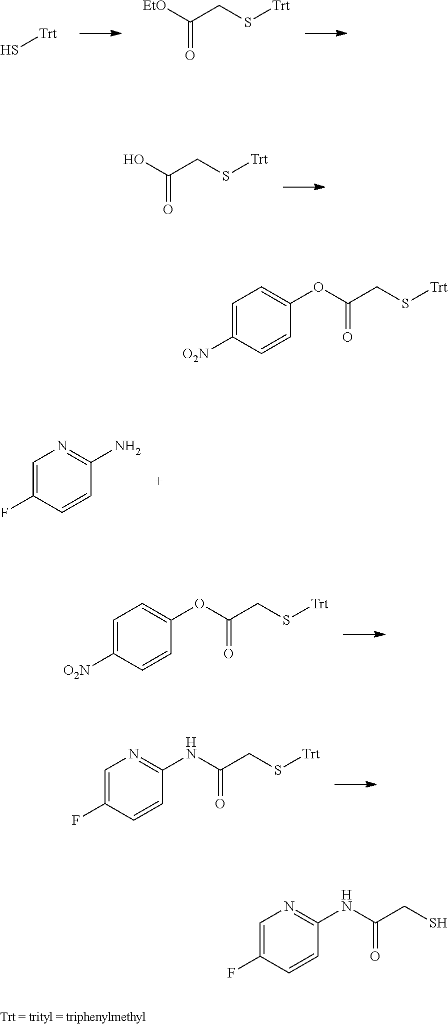

### Ethyl 2-(tritylthio)acetate

A solution of triphenylmethanethiol (20.0 g, 72.3 mmol), ethyl bromoacetate (8.83 mL, 79.6 mmol), diisopropylethylamine (15.1 mL, 86.8 mmol) and dimethylformamide (60 mL) was stirred at room temperature for 3 h. The reaction was diluted with ethyl acetate (300 mL) and washed with H₂O (3×80 mL), saturated aqueous citric acid (3×80 mL), saturated aqueous NaHCO₃ (3×80 mL), and brine (2×80 mL). The organic solution was dried (Na₂SO₄) and rotary-evaporated (30 min with a bath temperature of 45° C.) to remove the excess ethyl bromoacetate. High vacuum (1 mm Hg) was applied for 48 h to provide the desired product as light-yellow crystals (24.09 g, 92% yield).

### 2-(Tritylthio)acetic acid

A solution of ethyl 2-(tritylthio)acetate (24.09 g, 66.5 mmol), 2N NaOH (66 mL, 133 mmol), and dioxane (66 mL) was refluxed for 2 h. The reaction was cooled to room temperature and diluted with ethyl acetate (150 mL) and H₂O (150 mL). The basic aqueous layer was collected, and the organic layer was extracted once with H₂O (50 mL). The combined aqueous layers were made acidic with solid citric acid (˜40 g) with stirring to pH 2-4. The product was extracted with dichloromethane (3×80 mL), dried (Na₂SO₄) and rotary-evaporated to provide 20.49 g, 92.1% yield of a white solid.

### 4-Nitrophenyl-2-(tritylthio)acetate

A solution of 2-(tritylthio)acetate (20.49 g, 61.3 mmol), 4-nitrophenol (10.2 g, 73.5 mmol), N-Ethyl-N′-(3-dimethylaminopropyl)carbodiimide hydrochloride (12.9 g, 67.4 mmol) in ethyl acetate (200 mL), dichloromethane (100 mL), and dimethylformamide (50 mL) was stirred 3 days under argon at room temperature. The reaction was diluted with diethyl ether (400 mL) and washed with saturated aqueous citric acid (3×100 mL), saturated aqueous NaHCO₃ (3×100 mL), saturated aqueous K₂CO₃ (100 mL), and brine (100 mL). The organic layer was then dried (Na₂SO₄) and rotary-evaporated to a yellow solid. This material was further purified by silica gel chromatography, gradient elution from hexane to 4:1 ethylacetate:hexane to provide 18.81 g, 67% yield of a faintly yellow powder that was subsequently stored under argon.

### N-(5-fluoropyridin-2-yl)-2-(tritylthio)acetamide

To a solution of 2-amino-5-fluoropyridine (235.4 mg, 2.1 mmol) and 4-nitrophenyl-2-(tritylthio)acetate (957.6 mg, 2.1 mmol) in DMF (10 mL) was added triethylamine (725 μL, 4.2 mmol). The reaction was stirred for 18 h at 50 C, and the reaction mixture was poured into diethyl ether (100 mL), washed with 2N NaOH (4×20 mL), H₂O (2×20 mL), and brine (20 mL). The organic layer was dried (Na₂SO₄) and rotary-evaporated to give 674 mg, 75% yield of a solid.

### N-(5-fluoropyridin-2-yl)-2-mercaptoacetamide

Under argon tri-isopropyl silane (500 μL, 4.08 mmol) was added to a solution of N-(5-fluoropyridin-2-yl)-2-(tritylthio)acetamide (674 mg, 1.58 mmol) dissolved in dichloromethane (10 mL). Trifluoroacetic acid (10 mL) was added slowly over 5 min., and the reaction was stirred at room temperature for 15 min. after the addition was complete. The volatiles were removed on the rotary-evaporator, and the crude product was votexed twice with hexane (5 mL) to remove the triphenylmethane byproduct. The insoluble product was collected on fritted glass and washed with hexane (5 mL) to provide 291 mg, 99% yield of a powder.

### Example 14

### Synthesis of N-(2,4-dichlorobenzyl)-2-mercaptoacetamide

An alternative route to mercaptoacetamide analogs is a three step procedure in which an amine is first reacted with chloroacetylchloride. The resulting chloride is then reacted with potassium thioacetate. The mercaptoacetamide is formed after aqueous hydrolysis of the thioacetate ester.

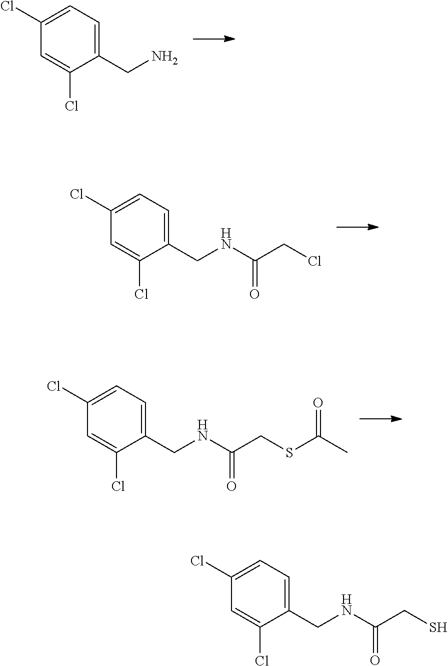

### N-(2,4-Dichlorobenzyl)-2-chloroacetamide

A solution of 2,4-dichlorobenzylamine (352 mg, 2.0 mmol) in THF (5 mL) was cooled in an ice bath, and chloroacetyl chloride (191 μL, 2.4 mmol) was added followed by the dropwise addition of triethylamine (418 μL, 3.0 mmol). The reaction was tested for completion using Ninhydrin spray reagent on a TLC plate. The reaction was quenched by the addition of 1N HCl (10 mL) and ethyl acetate (50 mL). The organic layer was washed with 1N HCl (3×10 mL), saturated aqueous NaHCO₃ (2×10 mL), and brine (10 mL). The organic layer was then washed (Na₂SO₄) and dried under high vacuum to provide 444.6 mg, 88% yield of a tan solid.

### S-(2,4-Dichlorobenzylcarbamoyl)methyl ethanethioate

To a solution of N-(2,4-dichlorobenzyl)-2-chloroacetamide (440 mg, 1.74 mmol) in DMF (3 mL) was added potassium thioacetate (398 mg, 3.48 mmol). The reaction mixture was heated to reflux for 1 min. and cooled to room temperature. The reaction was diluted with ethyl acetate (50 mL) and washed with saturated aqueous NaCl (2×20 mL), saturated aqueous citric acid (2×20 mL), and brine (20 mL). The organic layer was dried (Na₂SO₄), rotary-evaporated, and chromatographed on silica gel, gradient elution with 25% ethyl acetate/hexane to 50% ethyl acetate/hexane to provide 353 mg (70% yield) of a pink solid. NMR was consistent with the product.

### N-(2,4-dichlorobenzyl)-2-mercaptoacetamide

A solution of S-(2,4-dichlorobenzylcarbamoyl)methyl ethanethioate (150 mg, 0.517 mmol) was dissolved in methanol (4 mL), and the solution was repeatedly degassed with vacuum/argon. An aqueous solution of 2N NaOH (1.3 mL, 2.58 mmol) was added through a septum and the reaction mixture was stirred at room temperature for 30 min. to cleave the acetate ester. The reaction was then quenched with 1N HCl (3.0 mL) while still under an inert atmosphere. The methanol was evaporated under vacuum and the resulting solution was extracted with dichloromethane (2×5 mL). The organic layer was washed with brine (2 mL), dried over Na₂SO₄ and evaporated under vacuum to give 122.5 mg (95% yield) of the desired mercaptoacetamide as a solid.

### Example 15

### Enzyme Assay

Compounds of the invention were tested for ability to inhibit botulinum toxin in an enzyme assay as shown in Schmidt et al. Applied and Environmental Microbiology, 659(1):297-303, 2003.

### Peptide Synthesis

The Nε-(2,4-dinitropheyl)-lysine peptide was purchased from American Peptide Company (Sunnyvale, Calif.). The fluorophore was then introduced by reacting the sulfhydryl group of the cysteine residue with 3-iodoacetamido-4-methyl-7-dimethylamino-coumarin (DACIA; Molecular Probes, Inc., Eugene, Oreg.) as follows.

Solutions of 2 to 5 mM peptide and 10 mM DACIA were prepared in methyl sulfoxide. Equal volumes of each were mixed, and N,N-diisopropylethylamine was added to approximately 30 mM. Peptide reacted with the reagent in 30 min at ambient temperature. Excess reagent was then discharged by adding mercaptoethanol to 0.05 M. After another 20 to 30 min, the reaction mixture was diluted with 8 volumes of water, and the pH was adjusted to about 2 with TFA. The product was concentrated by solid-phase extraction (C18 Sep-Pak; Waters Corp.) and purified by reverse-phase HPLC. The reaction product of DACIA and the cysteine sulfhydryl group is abbreviated as “daciaC.”

### Assays of Botulinum Toxin Protease Activities

Fluorescence spectra were obtained with a Molecular Devices Gemini XS plate reader (Sunnyvale, Calif., now a division of MDS Analytical Technologies). Fluorescence was expressed in arbitrary units. In the assays, buffer (40 mM HEPES-0.05% Tween [pH 7.3]) and substrate were mixed in a volume of 80 μL and placed in a 96-well plate. The reaction was started by the addition of recombinant light chain. Fluorescence was monitored at 1 point per s. The excitation and emission wavelengths were 398 and 485 nm, respectively. The temperature was maintained at 25 to 27° C.

Kinetic constants were obtained from plots of initial rates versus seven concentrations of substrate. Results were calculated from nonlinear regression analyses by using the program Enzfitter (Biosoft, Cambridge, United Kingdom). The values are the averages of three independent determinations±standard deviation.

The Ki value for the compounds of the invention were first calculated from a Dixon plot

by using the equation Ki=Km/[(slope)(Vmax)(S)], where S is the substrate concentration. The Ki value and the type of inhibition (competitive, noncompetitive, etc.) were then determined by calculation of substrate Vmax and apparent Km (Knapp), as described above, in the presence of three different inhibitor concentrations for each compound. Ki values for each of the three assays in the presence of inhibitor were calculated with the equation Ki=[I]/[(Kmapp/Km)−1], where [I] is the inhibitor concentration.

### Results

After incubating the substrate with compounds of the invention and recombinant light chain, intact substrate was observed. Thus compounds of the invention were found to bind to the recombinant light chain and inhibit hydrolysis of substrate. Table 1 below shows enzyme inhibition of select compounds.

TABLE 1
Inhibition of enzyme activity for select compounds of the invention

| Chemical Structure | Enzyme Inhibition IC50 (uM) |
|---|---|
| 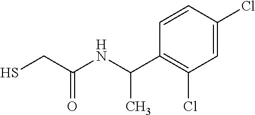 | 5-27 |
| 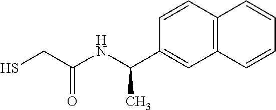 | 8-21 |
| 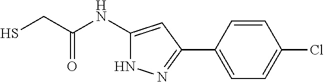 | 3-7 |
| 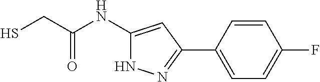 | 12-15 |

### Example 16

### Cellular Assay

Compounds of the invention were tested for ability to inhibit botulinum toxin in a cellular assay as shown in Skaper et al., Develop NeuroSci, 2:233-237, 1979; and Foran et al., The Journal of Biological Chemistry, 278(2):1363-1371, 2003.

### Primary Rat Cerebellar Neuron-Based Assay

Neuronal granule cells from pooled cerebellar of 7-8 day old Sprague-Dawley rats were obtained by the methods described by Skaper et al. (Develop. Neurosci., 2:233, 1979). Cells were suspended in DMEM/F-12 medium (Gibco-Invitrogen, Carlsbad, Calif.) supplemented with 50 U/mL penicillin, 50 μg/mL streptomycin, 10% FBS, N2 supplement, and 25 mM KCl. Cells were seeded onto poly(L-lysine)-coated 6-well plates at a density of 2.2×10⁶ cells per well and were maintained in a humidified 5% CO₂ atmosphere at 37° C. After 24 hours, 10 μM cytosine-B-D-arabinoside (Sigma, St. Louis, Mo.) was added to the culture to inhibit the replication of non-neuronal cells. The neurons were maintained by replacement every 7 days with the same freshly prepared medium.

The primary neuronal cell culture are well established usually within 8-10 days after initial seeding and remains useful for up to four weeks. Varying concentrations of compounds of the invention, dissolved in DMSO, were added to the culture, subsequently followed by addition of 0.5 nM botulinum (serotype-A, Allegran, Inc., Irvine, Calif.). After receiving the botulinum inoculation, the cultures were allowed to incubate at 37° C., 5% CO₂, for 3 hours. The cells were washed with PBS and harvested into a pre-weighed eppendorf screw cap vial. The cells were then pelleted by centrifugation. After pelleting, the supernatant was carefully removed and the microcentrifuge tubes were weighed to determine the weight of the pellet. 4 μl of M-PER, and 2 μl of SDS-PAGE sample buffer were added for each mg of cell pellet, and boiled for 10 minutes to inactivate the residual toxin. The samples were subjected to immunoblot analysis.

### Serum Neutralization Test Against BoNT/A

Cells were seeded into a 6-well tissue culture plate and allowed to grow with a change to fresh culture medium as described above. Serial dilutions of scFv were made in PBS to a final volume of 0.1 ml and were incubated for 30 min at room temperature with 0.1 nM botulinum. The reaction mixtures were added to these plates and incubated for 3 h at 37° C. in a humidified atmosphere containing 5% CO₂. The cells were washed with DPBS three times and harvested. Neutralization titers were expressed as the reciprocal of the highest dilution that inhibited SNAP-25 cleavage in M17 cells by BoNT/A.

### Immunoblot Analysis

Aliquots of protein were electrophoresed through 15% polyacrylamide gels. Proteins were transferred to polyvinylidene fluoride membranes (Millipore Corp., Bedford, Mass.) and processed for immunoblot analysis. Block was performed with 5% nonfat dry milk in TBST (10 mM Tris, 150 mM NaCl, 0.1% (v/v) Tween 20) for 1 h at room temperature and blots were probed with rabbit anti-SNAP-25 antibody (1:2500, Sigma, St. Louis, Mo.) in 1% nonfat dry milk in TBST for 1 h at room temperature. Following washes with TBST, secondary antibody incubation was with HRP-conjugated goat anti-Rabbit IgG (1:10,000, Amersham Biosciences) in 1% nonfat dry milk in TBST. After 1 h at room temperature, blots were washed and developed using ECL kit (GE Healthcare Bio-Sciences Corp.). Band densities for SNAP-25 and its cleaved product were normalized and relative intensity was determined using scanning densitometry (Kodak Image station 200RT, Eastman Kodak Company, Rochester, N.Y.).

### Results

Table 2 shows cellular inhibition of select compounds.

TABLE 2
Inhibition in cellular assay for select compounds of the invention

| | Cell Biology (% | Cell Biology |
|---|---|---|
| Chemical Structure | inhibition at 30 uM) | IC50 (uM) |
| 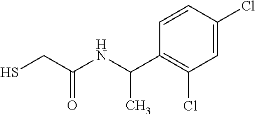 | — | 18 |
| 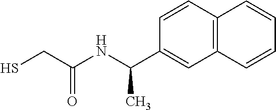 | — | — |
| 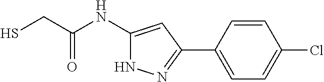 | 57-100% | 10-19 uM |
| 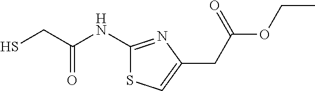 | 86% | — |
| 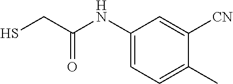 | 77% | — |
| 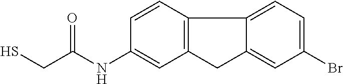 | 90 | — |
| 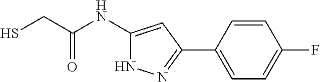 | 73-78% | — |

What is claimed is:

1. A method for treating a botulinum toxin related condition comprising administering to a subject in need thereof a therapeutically effective amount of a pharmaceutical composition comprising a mercaptoacetamide.

2. The method according to claim 1, wherein the botulinum toxin is a serotype selected from the group consisting of: serotype A, serotype B, serotype C, serotype D, serotype E, serotype F, and serotype G.

3. The method according to claim 1, wherein the subject is human.

4. The method according to claim 1, wherein the botulinum toxin related condition is botulism.

5. The method according to claim 1, wherein the mercaptoacetamide is a compound of formula I: 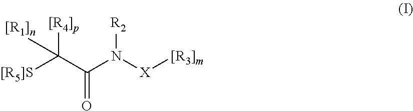
wherein:
X is a C₃-C₆ heterocycloalkenyl, wherein carbon atoms of the ring are optionally substituted by R₆, and wherein when one or more heteroatoms are nitrogen, the nitrogens are each independently unsubstituted or substituted by R₇;
R₁ is present at n occurrences, n is an integer from 0 to 1, and R₁ is selected from H, C₂-C₆ alkenyl, C₂-C₆ alkynyl, C(═O)OR₉, and C₁-C₆ alkyl optionally substituted by R₈;
R₂ is selected from H, C₂-C₆ alkenyl, C₂-C₆ alkynyl, C(═O)OR₉, and C₁-C₆ alkyl optionally substituted by R₈;
R₃ is present at m occurrences, m is an integer from 0 to 1, and R₃ is selected from a proton, C(═O)ORᵢₒ, C(═O)OR₇, C(═O)NR₇, C₃-C₆ heterocycloalkylaryl, C₃-C₆ cycloalkenylaryl, C—R₈, heteroaryl, and aryl optionally substituted at each carbon atom by halo, OH, OCH₃, O-alkyl, amino, substituted amino, —SO₂NH₂, substituted sulfonamide, —SO₂CH₃, substitutied sulfoxies, CONH₂, substituted amido, COCH₃, substituted ketones, CHO, cyano, NO₂, C(═O)OR₁₀, and C₁-C₆ alkyl which is further optionally substituted by halo, amino, or hydroxyl;
R₄ is present at p occurrences, p is an integer from 0 to 1, and R₄ is selected from H, C₂-C₆ alkenyl, C₂-C₆ alkynyl, C(═O)OR₉, and C₁-C₆ alkyl optionally substituted by R₈;
R₅ is a hydrogen atom, or a bond such that the molecule formed a symmetrical dimer at the disulfide bond, a mixed disulfide with other monosulfide compounds such as ethanethiol, or functional groups such as acetyl to form esters which can be used as prodrugs, H, —C(═O)R₉, and —C(═O)OR₉;
R₆ is selected from H, C₂-C₆ alkenyl, C₂-C₆ alkynyl, C(═O)OR₉, and C₁-C₆ alkyl optionally substituted by R₈;
R₇ is selected from H, C₁-C₆ alkyl optionally substituted by R₈; C₂-C₆ alkenyl, C₂-C₆ alkynyl, C(═O)OR₉, and aryl optionally substituted by halo or C₁-C₆ alkyl;
R₈ is selected from C(═O)OR₉, OR₉, and halo;
R₉ is a C₁-C₆ alkyl optionally substituted by aryl;
R₁₀ is selected from H, halo, OR₉, NO₂, alkoxy, cyano, SO₂CH₃, SO₂NH₂, COCH₃, COCH₃, CONH₂, CHO and C₁-C₆ alkyl optionally substituted by halo;
R₁₁ is an aryl optionally substituted by halo; or
pharmaceutically acceptable salts and prodrugs thereof.

6. The method according to claim 5, wherein:
X is a C₃-C₆ heterocycloalkenyl, wherein carbon atoms of the ring are optionally substituted by R₆, and wherein when one or more heteroatoms are nitrogen, the nitrogens are each independently unsubstituted or substituted by R₇;
R₁ and R₂ are independently selected from H or methyl;
R₃ is selected from a proton, C(═O)OR₁₀, C(═O)OR₇, C(═O)NR₇, C₃-C₆ heterocycloalkylaryl, C₃-C₆ cycloalkenylaryl, C—R₈, heteroaryl, and aryl optionally substituted at each carbon atom by halo, OH, OCH₃, O-alkyl, amino, substituted amino, —SO₂NH₂, substituted sulfonamide, —SO₂CH₃, substitutied sulfoxies, CONH₂, substituted amido, COCH₃, substituted ketones, CHO, cyano, NO₂, C(═O)OR₁₀, and C₁-C₆ alkyl which is further optionally substituted by halo, amino, or hydroxyl;
R₆ and R₇ are independently selected from H or methyl; or
pharmaceutically acceptable salt and prodrugs thereof.

7. The method according to claim 6, wherein X is at least one compound selected from the group consisting of pyrazole, thiazole, and thiadiazole.

8. The method according to claim 7, wherein the pyrazole is a 1,2 pyrazole.

9. The method according to claim 7, wherein the thiazole is a 1,3 thiazole.

10. The method according to claim 7, wherein the thiadiazole is a 4-thia-1,2 diazole.

11. The method according to claim 6, wherein R₁ and R₂ are both H.

12. The method according to claim 6, wherein R₆ and R₇ are both H.

13. The method according to claim 6, wherein R₃ is at least one compound selected from the group consisting of phenyl, furyl, pyridyl and thiophene.

14. The method according to claim 13, wherein thiophene is 2-thiophene.

15. The method according to claim 1, wherein the mercaptoacetamide is a compound of formula II: 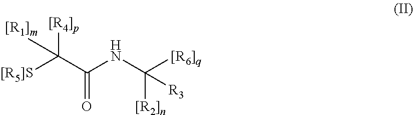
wherein:
R₁ is present at m occurrences, m is an integer from 0 to 1, and R₁ is C₁-C₆ alkyl or C—R₈,
R₂ is present at n occurrences, n is an integer from 0 to 1, and R₂ is selected from a proton, C₁-C₆ alkyl, C(═O)OR₇, alkyl-OR_{S}, C—R₈, and alkyl-NR₉;
R₃ is selected from a proton, C(═O)OR₁₀, C(═O)OR₇, C(═O)NR₇, C₃-C₆ heterocycloalkylaryl, C₃-C₆ cycloalkenylaryl, C—R₈, heteroaryl, and aryl optionally substituted at each carbon atom by halo, OH, OCH₃, O-alkyl, amino, substituted amino, —SO₂NH₂, substituted sulfonamide, —SO₂CH₃, substitutied sulfoxies, CONH₂, substituted amido, COCH₃, substituted ketones, CHO, cyano, NO₂, C(═O)OR₁₀, and C₁-C₆ alkyl which is further optionally substituted by halo, amino, or hydroxyl;
R₄ is present at p occurrences, p is an integer from 0 to 1, and R₄ is C₁-C₆ alkyl or C—R₈;
R₅ is a hydrogen atom, or a bond such that the molecule formed a symmetrical dimer at the disulfide bond, a mixed disulfide with other monosulfide compounds such as ethanethiol, or functional groups such as acetyl to form esters which can be used as prodrugs, H, and —C(═O)R₁₀;
R₆ is present at q occurrences, q is an integer from 0 to 1, and R₆ is aryl;
R₇ is selected from C—R₈;
R₈ is selected from C₃-C₆ cycloalkenylaryl, C₃-C₆ heterocycloalkenylaryl, and aryl optionally substituted by OH, aryl, or OR₁₀
R₉ is C(═O)OR₁₀;
R₁₀ is C₁-C₆ alkyl; or
pharmaceutically acceptable salts and prodrugs thereof.

16. The method according to claim 1, wherein the mercaptoacetamide is a compound of formula III: 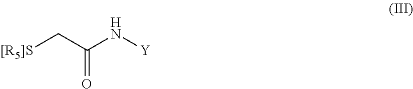
wherein:
Y is selected from C₁-C₆ alkyl, C₂-C₆ alkenyl, C₃-C₆ cycloalkenylaryl, C₃-C₆ heterocycloalkenylaryl, C₃-C₆ cycloalkylaryl, C₃-C₆ heterocycloalkylaryl, aryl, heteroaryl, C₃-C₆ heterocycloalkenyl, C₃-C₆ arylcycloalkylaryl, any of which is optionally substituted at each carbon atom by R₁, and wherein when one or more heteroatoms are nitrogen, the nitrogens are each independently unsubstituted or substituted by R₂;
R₁ is selected from OH, cyano, SH, halo, alkyl-NR₂R₃, OR₂, aryl, oxo, C—R₃, OR₃, C₂-C₆ alkynyl, C₃-C₆ heterocycloalkenylaryl, C₁-C₆ alkyl optionally substituted by halo; and C₃-C₆ heterocycloalkenyl which is optionally substituted at each carbon atom by R₄;
R₂ is selected from C₁-C₆ alkyl;
R₃ is selected from a proton, C(═O)OR₁₀, C(═O)OR₇, C(═O)NR₇, C₃-C₆ heterocycloalkylaryl, C₃-C₆ cycloalkenylaryl, C—R₈, heteroaryl, and aryl optionally substituted at each carbon atom by halo, OH, OCH₃, O-alkyl, amino, substituted amino, —SO₂NH₂, substituted sulfonamide, —SO₂CH₃, substitutied sulfoxies, CONH2, substituted amido, COCH3, substituted ketones, CHO, cyano, NO₂, C(═O)OR₁₀, and C₁-C₆ alkyl which is further optionally substituted by halo, amino, or hydroxyl;
R₄ is C(═O)OR₂;
R₅ is a hydrogen atom, or a bond such that the molecule formed a symmetrical dimer at the disulfide bond, a mixed disulfide with other monosulfide compounds such as ethanethiol, or functional groups such as acetyl to form esters which can be used as prodrugs; or
pharmaceutically acceptable salts and prodrugs thereof.

17. The method according to any of claim 5, 15, or 16 wherein, R₅ is a bond such that the molecule formed a symmetrical dimer at the disulfide bond, a mixed disulfide with other monosulfide compounds such as ethanethiol.

18. The method according to claim 1, wherein the mercaptoacetamide is a compound selected from the group consisting of:

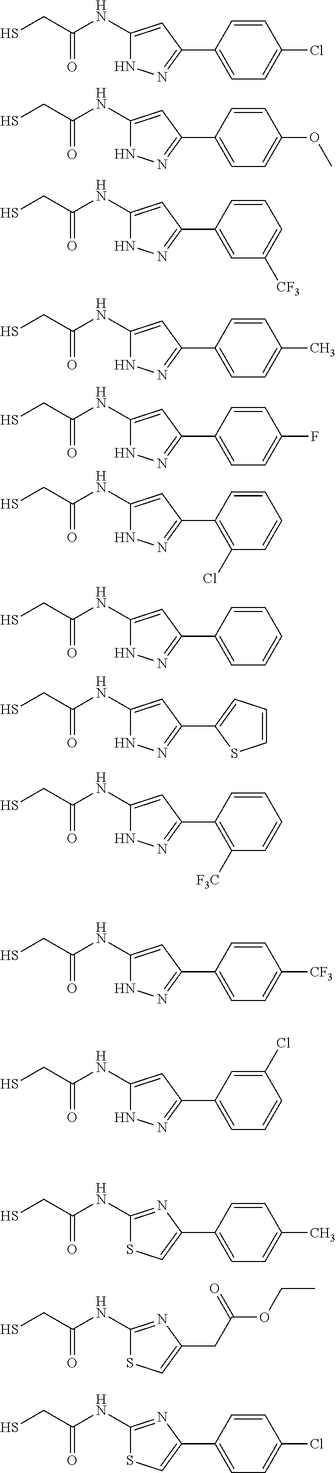

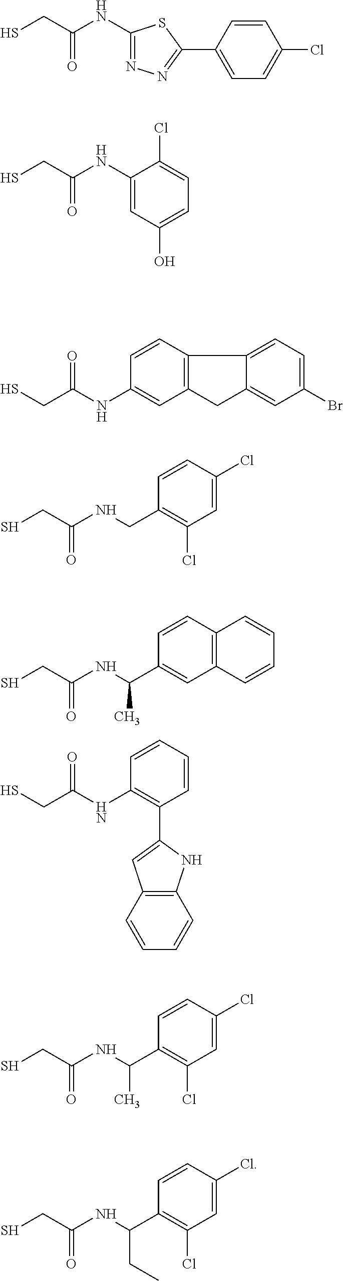

19. A method for treating a botulinum toxin related condition comprising administering to a subject in need thereof a therapeutically effective amount of a pharmaceutical composition comprising a compound of formula IV: 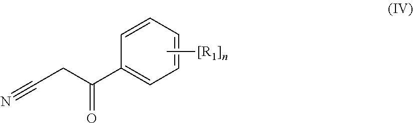
wherein:
R₁ is present at n occurrences, n is an integer from 0 to 5 and R₁ is selected from halo and C₁-C₆ alkyl optionally substituted by halo.

20. A method for treating a botulinum toxin related condition comprising administering to a subject in need thereof a therapeutically effective amount of a pharmaceutical composition comprising a compound of formula V: 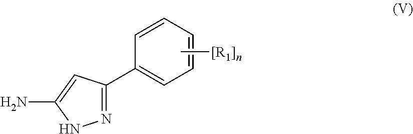
wherein:
R₁ is present at n occurrences, n is an integer from 0 to 5 and R₁ is selected from halo and C₁-C₆ alkyl optionally substituted by halo.

21. A pharmaceutical composition comprising a mercaptoacetamide in a dosage effective to treat a botulinum toxin related condition, and a pharmaceutically acceptable carrier.
